(19) United States
(12) Patent Application Publication
(10) Pub. No.: US 2025/0016245 A1
(43) Pub. Date: Jan. 9, 2025

(54) COLLECTION, SECURE STORAGE, ANALYSIS, PERMISSIONING, SHARING, AND DISPLAY OF STREAMING AND/OR STATIC PERSONAL DATA

(71) Applicant: Fulcra Dynamics Corporation, Portsmouth, NH (US)

(72) Inventors: Michael J.J. Tiffany, Dover, NH (US); Brandon James Creighton, San Diego, CA (US); Matthew G. Harrigan, San Diego, CA (US); Ashur Barrett Kalb, Warwick, NY (US); Diana Sachiko Kusunoki, Holualoa, HI (US)

(21) Appl. No.: 18/757,092

(22) Filed: Jun. 27, 2024

Related U.S. Application Data
(60) Provisional application No. 63/525,218, filed on Jul. 6, 2023.

(51) Int. Cl. H04L 67/565; H04L 67/50

(52) U.S. Cl. CPC H04L 67/565; H04L 67/535

(57) ABSTRACT

According to various embodiments, time-varying data describing aspects of a user's life may be collected from a plurality of sources and displayed in a unified timeline. The timeline may present such data from disparate sources within a common time-based framework. The timeline may be interactive, allowing the user to manipulate the timeline display, by for example moving a playhead along an axis; the data display may be automatically updated to show data for the time period associated with the position of the playhead. In various embodiments, data connections with disparate sources may be established, so as to allow the collection, manipulation, display, and secure storage of multiple types of data from multiple sources, wherein such display is unified, interactive, and well-adapted to facilitate comparison of data from the multiple sources.

### CROSS-REFERENCE TO RELATED APPLICATION

This application claims the benefit of U.S. Provisional Patent Application Ser. No. 63/525,218, filed on Jul. 6, 2023 and entitled “Collection, Secure Storage, Analysis, Permissioning, Sharing, and Display of Streaming and/or Static Personal Data,” which is incorporated by reference as though set forth herein in its entirety.

### TECHNICAL FIELD

The present document relates to systems and methods for collecting, securely storing, analyzing, permissioning, sharing, normalizing, and displaying streaming and/or static personal data.

### BACKGROUND

Increasingly, software from various sources does not work well together, with each individual software component or architecture, and the data contained therein, being siloed. Users who wish to integrate data from various sources must expend significant amounts of their own efforts to make such integration happen.

Existing technological tools are limited to storing aspects of a user's life in a manner that can be analogized to a series of spreadsheets, which may yield limited actionable results for most people.

### SUMMARY

According to various embodiments, the techniques described herein implement and/or provide a personal operating system that is able to turn previously siloed personal data into a high-fidelity digital twin, so as to empower people with the use of data science and automation, as well as to make that data available to tools such as AI agents, in a cross-platform manner.

In at least one embodiment, the described system may operate as a hub for all the data produced in connection with a user's life, such as data obtained by sources that may include software as a service (SaaS), wearables, computers, augmented reality (AR) devices, virtual reality (VR) devices, and/or Internet of Things (IoT) devices. By placing such data under the user's control, the described system facilitates application of data science and/or machine learning to such data. In addition, the described system may generate shared awareness dashboards and notifications for the user, as well as other interested parties such as the user's staff and/or coaches. The system may further facilitate the training of models on the data for prediction or experimentation in progress. The system may further make the data available to be queried by tools such as AI agents via an API.

In at least one embodiment, the system may be implemented in the context of a trustworthy platform for various streams of data produced in connection with a user's life. Data concerning a user may be automatically de-siloed so as to automate more of the workflows associated with the user. The system may be made capable of absorbing more varied data, so as to enable the application of the described techniques to drive health, performance optimization, and/or productivity analytics in a user-friendly dashboard and in a data science notebook.

The de-siloing and normalization performed by the system may also be used for training a model on the data associated with the user's life, and/or for making the data available to be queried by a model that knows how to make use of the data.

In at least one embodiment, the system can provide new and innovative ways to visualize certain types of personal data, such as via a timeline/dashboard or data science notebook, and can also provide a customizable service layer to design experiments and report analysis and results on a per-user basis.

In at least one embodiment, the system may be implemented as a software platform. Such a platform may be used to create a functional translation layer that takes all manner of personal data, analyzes it, transforms and/or normalizes it, and makes it available to any number of computing and interaction platforms. Such personal data may be collected from any suitable source, and may include, for example, fitness data, finances data, data concerning how a user spends their time, web browsing data, and/or the like.

In at least one embodiment, the described system provides a repository for all the personal data generated by a user and/or associated with a user as they live their life. Much of this data, such as user location, media consumption, health data, and/or the like, can take the form of a data stream. By bringing data streams together and tracking relationships among such various data sources in a stream-based form, the system is able to enable functionality for exploring causal and/or correlative relationships and thereby provide insights into the effect of such data streams on each other. In addition, storing such data in a streaming form improves security, because it allows a user to combine multiple data sources without giving personal data from one source to another.

One issue that may exist with conventional file storage mechanisms is that such files may be compromised or stolen by malicious actors, whether the files are stored locally or in the cloud. By providing a trustworthy platform for aggregation of streaming data, the system described herein allows for more secure storage by incorporating modern breakthroughs in differential privacy and security risk mitigation. In at least one embodiment, the system may be configured to only provide the specific data necessary to carry out any particular query and/or action, thereby avoiding the need to upload a file containing more data than is necessary.

In at least one embodiment, the described system provides functionality to allow users to perform any or all of the following functions, so as to enable such users to live a more deliberate life:
Free their data from silos;
Measure what matters to them;
Make data-driven decisions;
Provide visibility to certain data streams or analyses to the stakeholders in their lives;
Share awareness about their lives;
Delegate more effectively; and/or
Automate what can be automated based on the foregoing.

In various embodiments, the systems and methods described herein may be used in any context where it may be useful or desirable to securely collect, store, analyze, permission, share, and/or display streaming and/or static personal data, or to perform any subset of the above functions. The systems and methods described herein may be implemented, for example, using any appropriate platform, including for example any combination of computing, mobile, and/or other electronic device or devices.

In at least one embodiment, the system may use an app and/or other instrumentation as appropriate on a per-user basis, including, for example, wearables, stochastic polls, calendar driven polls, buttons (which may be physical or touchscreen-based) representing events (such as mood, headache, coffee, and/or the like), and/or other methods of passive and active data collection. In at least one embodiment, the system may connect to other data sources (such as financial APIs) to obtain additional streaming data.

In contrast to user-accessible exports of personal data streams as may be generated by other services, which may often take the form of batch exports delivered as files, the system described herein may keep and continuously update time series data. In at least one embodiment, such data streams may be received via an API; using the techniques described herein, such API-based data streams may be saved and/or rolled backwards as desired.

Access methods to the streaming data stored by the described system may, if desired, be limited in ways that may be impractical or impossible with conventional file storage mechanisms, thus providing security-enhancing options to prevent or encumber attackers attempting data discovery and exfiltration.

In contrast to conventional apps that may use personal data (such as, for example, location data embedded in images captured by a photo app), the described system may ask a user using a device to grant the app access to that data, for example by letting an image capture app have access to a device's location, either once, or when the app is running, or at all times. The system can thereby allow a user to correlate images with locations, and/or to allow other types of correlations to be established, without necessarily allowing the producer of one data stream to have access to the other. The apps may remain blind to each other's data, while the user can generate and view correlations.

The system may provide secure collection and storage of streaming personal data, unlike existing systems that are based on the abstract notion of files, not streams.

The system may provide functionality to augment streaming data with scheduled or stochastic polling. Data collection may be driven by user configuration (for example, to track a habit) and/or by analysis of streaming data (for example, by indicating to a user that the system noticed that their heart rate was elevated in a recent meeting, and asking the user what was going on). In this manner, the system is able to add difficult-to-measure data, such as subjective data (“How are you feeling?”) into an integrated time series including concrete, easy-to-measure data such as heart rate.

The system may provide navigable presentation of time series (and other) personal data in a dashboard (such as a timeline having a normalized, unified time scale). In at least one embodiment, such a display may include horizontal calendar and location visualization, allowing for situational awareness and pattern analysis.

The system may provide co-design of user experimentation and reporting, including streaming data and/or stochastic polls.

The system may provide a data-driven prospective and retrospective time analysis and blocking, so as to map otherwise un-tracked events (such as habits) based on stochastic polling, calendar analysis, location, streaming data analysis, and/or the like. For example, the system may indicate that it has noticed that the user's heart rate was elevated and their location was a gym; accordingly, the system may add a workout session to their timeline.

The system may provide a “signal strength” feature that indicates the status of the data streams of a user's life (such as events, heart rate, and/or the like) as well as their data sources (such as Apple Health and/or other apps) with observability metrics. The system may present to the user a display indicating the number of events across time and the last time data was received; warnings may be included to indicate when the user is disconnected so that data may not have been reliably collected. Integrations may also be tracked, including proportions of data sources and devices included in the data corpus, and a breakdown of categories of data integrations.

In at least one embodiment, the system provides a way to tap into the extraordinary volumes of data that users' devices are collecting about their lives. The system provides functionality to surface such data in useful ways, and to make such data programmatically accessible in a secure, private, permissions-based way.

In at least one embodiment, the system provides an orchestration layer and common data model, so as to enable cross-platform, cross-device data integration held together with an orchestration layer and a common data model. In this manner, the system is able to unify data in a fragmented real-world environment.

In at least one embodiment, a client's collected and collated personal data, which may be accessed via an API accessed using an API key created by the client, can be used to train or fine-tune mathematical models, which may be either predictive or generative. The weights and other technical data associated with a client's models can be stored securely in the same data store for later retrieval. Predictive models, such as ARIMA or various linear and non-linear regression models, may be used and visualized with other components on the Portal. Generative models may be used to react to future events in such a way as to mimic the client's own reaction (or an enhanced version thereof). For example, this may be used to recommend brief answers to text messages. Separately, the client's personal data, accessed in the same manner, may be integrated through third-party components and/or services to act as a source of truth for providing additional context and correct answers to generic ML models, such as large language models. This allows for another form of user interaction (UI) with a user's own data. In at least one embodiment, a geo-coding API may be used to turn latitude and longitude data into location names.

The system and method thus provide mechanisms for de-siloing and normalizing various types of data, and for presenting such data in a unified interactive timeline, which may include a calendar display. In at least one embodiment, data may be automatically cleaned and normalized, and may be presented to tools such as computational notebooks and Artificial Intelligence (AI) agents with consistent semantics, regardless of source. Finally, in at least one embodiment, a derived time series personal data can be presented as contextual data to an AI agent.

In at least one embodiment, the system allows for the use of custom inputs.

Further details are provided below.

### BRIEF DESCRIPTION OF THE DRAWINGS

The accompanying drawings, together with the description, illustrate several embodiments. One skilled in the art will recognize that the particular embodiments illustrated in the drawings are merely exemplary, and are not intended to limit scope.

FIG. 1 is a block diagram depicting a conceptual architecture for the described system, according to one embodiment.

FIG. 2 depicts an example of the integration of various components in an embodiment of the system, according to one embodiment.

FIG. 3 depicts an example of a timeline that may be presented via a timeline viewer interface according to one embodiment.

FIG. 4 depicts a navigation section of a timeline viewer interface, according to one embodiment.

FIG. 5 depicts a section of a timeline viewer interface for selecting date and time granularity, according to one embodiment.

FIG. 6 depicts a display in which the user's data may be played in a visual format, according to one embodiment.

FIGS. 7A through 7D depict the operation of a Side Panel that allows a user to quickly view more detailed information to explore their data, according to one embodiment.

FIG. 8 depicts an example of a display that may be presented by an Insights module, according to one embodiment.

FIG. 9 depicts an example of a display that may be presented by a Metascore module, according to one embodiment.

FIG. 10 depicts an example of a set of displays that may be presented by a Goals and Success Metrics module, according to one embodiment.

FIG. 11 depicts an example of a display that may be presented by a Visualization module in connection with goals and success metrics, according to one embodiment.

FIGS. 12A through 12E depict examples of a display that may be presented by an Events module, according to one embodiment.

FIGS. 13A through 13C depict examples of displays for Unscheduled Event Detection, according to one embodiment.

FIG. 14 depicts an example of a display for a Signal Strength module, according to one embodiment.

FIG. 15 is block diagram depicting a functional architecture for implementing the described system, according to one embodiment.

FIG. 16 depicts an example of a cohesive information architecture for the system, according to one embodiment.

FIG. 17 depicts an example of a platform for implementing the described system, according to one embodiment.

FIG. 18 depicts an example of a data flow diagram, according to one embodiment.

FIG. 19 depicts an example of an overall architecture for implementing the described system according to one embodiment.

FIGS. 20A through 20I depict screens for presenting insights according to one embodiment.

FIGS. 21A through 21F depict examples of various visualizations that may be available via an Explore Tab, according to one embodiment.

FIGS. 22A through 22C depict examples of various screens that may be associated with a Notifications Tab, according to one embodiment.

FIGS. 23A through 23D depict examples of various screens that may be associated with a Reflections Tab, according to one embodiment.

FIGS. 24A through 24H depict examples of various screens that may be associated with functionality for creating custom reflections according to one embodiment.

FIGS. 25A through 25F depict examples of various screens that may be associated functionality for customizing survey questions according to one embodiment.

FIGS. 26A through 26F depict examples of various screens that may be associated with functionality for configuring notifications and integrations according to one embodiment.

FIG. 26G depicts examples of various screens that may be associated with functionality for installing and configuring a practice according to one embodiment.

FIG. 26H depicts examples of screens for selecting and configuring a supporting routine according to one embodiment.

FIG. 26I depicts examples of screens for selecting and configuring a workout according to one embodiment.

FIGS. 27A through 27Z and FIGS. 28A through 28B depict examples of various screens that may be provided to enable functionality for meeting reflections according to one embodiment.

FIG. 29 is a block diagram depicting a hardware architecture for implementing the techniques described herein according to one embodiment.

FIG. 30 is a block diagram depicting a hardware architecture for implementing the techniques described herein in a client/server environment, according to one embodiment.

FIG. 31 is flow diagram depicting a method for generating and displaying a unified timeline view, according to one embodiment.

FIGS. 32A through 32D depict examples of a timeline display and associated displays, according to one embodiment.

FIG. 33A depicts an example of a Manage Connections screen, according to one embodiment.

FIG. 33B depicts an example of a Connections Details screen, according to one embodiment.

FIG. 34A depicts an example of a Bulk Selection screen, according to one embodiment.

FIG. 34B depicts an example of a Single Selection screen, according to one embodiment.

FIG. 35 depicts an example of a profile page, according to one embodiment.

FIG. 36 depicts an example of a Select Calendars screen, according to one embodiment.

FIG. 37 depicts an example of a Calendar Events screen, according to one embodiment.

FIG. 38 depicts an example of a Location screen, according to one embodiment.

FIG. 39 depicts an example of a Metrics screen, according to one embodiment.

FIGS. 40A through 40F depict examples of displays including various metrics presented across various time spans, according to one embodiment.

FIG. 41 depicts an example of a display of net calories, according to one embodiment.

FIG. 42 depicts an example of a summary display, according to one embodiment.

FIGS. 43A and 43B depict examples of sleep data displays, according to one embodiment.

FIG. 44A depicts an example of a main summary display, according to one embodiment.

FIG. 44B depicts an example of a mobile display including visualizations, according to one embodiment.

FIG. 44C depicts an example of a mobile display including metric tracks, according to one embodiment.

FIG. 45 depicts an example of a screen for selecting and/or deselecting metric tracks that are visible on timeline, according to one embodiment.

FIGS. 46A and 46B depict examples of screens for creating and managing custom inputs according to one embodiment.

FIGS. 47A through 47G depict examples of screens for creating and managing shares according to one embodiment.

### DETAILED DESCRIPTION OF THE EMBODIMENTS

### Glossary

For purposes of the description provided herein, certain terms are defined as follows:

### Admin Panel

An internal-only interface that allows team members to configure and customize the app, Timeline, Explorer, and Integrations to user's 100 needs.

### API Integrations

In at least one embodiment, the product suite may include API integrations:
With third-party solutions (e.g., Apple Health Kit, IFTTT)
Used by team members (e.g., ones to conduct analyses)
That support features that users 100 interact with (e.g., Flic button to record mood and various events)
consumable by automated observability tools
consumable by automated (e.g. AI) agents

### Biz Ops

Internal support tools for managing team and user performance.

### Community (of Practice)

Through their membership, users 100 gain access to a community of practice composed of like-minded individuals striving to live deliberately. This community allows users 100 to share their experiences about what worked for them and feel a sense of belonging. In at least one embodiment, users 100 can access a digital space where they can interact with other community members via the Portal.

Alternatively, the system may be integrated into third party AI and/or wellness apps to provide such apps with useful context to improve results. In such an environment, a coach may be a human, or may be an AI therapist, trainer, life coach, or other agent.

### Correlation Designer

An internal-only tool that enables the team to discover correlations in user's 100 data, especially unexpected ones, to surface to user 100 in Situation Reports or in insights on Timeline or Explorer. This tool can draw on datasets defined by analytical tools such as Notebooks created for user 100.

### Dashboard

An application that allows users 100 to view their life in a timeline format, explore correlations more in depth with Explorer, and/or dig into team findings in custom Notebooks. This application may be web-based and/or it may be a mobile app.

### DataPumphouse

DataPumphouse is a component that performs passive data collection by sending permissioned data generated by user's 100 phone to the system, requiring minimal user interaction with the interface. In at least one embodiment, user 100 does not interact with this directly. In other embodiments, user 100 may interact with this component, for example via an uploads counter.

### Experiences

The system provides unique experiences in the world to users 100 that allow them to internalize and expand their learnings from the experiments towards their goals. They can also help create rapport between user 100, their community, and the team. In at least one embodiment, users 100 can access experience information via the Portal.

### Experiments Runbook

Experiments with desired data, methodology, and analyses may be custom designed by Forward Deployed Engineers (FDEs) based on user-specific goals, and then placed in a document called a runbook which may be shared with user 100 for reference.

### Explorer

A Dashboard query building tool that allows user 100 to view and/or interact with their multi-dimensional data and dig further into insights created by the team or other correlations they may be interested in. It is an alternative analysis tool for looking at the same data presented within the timeline, but is not limited to presenting data according to time. An Explorer View is a saved configuration of datasets, queries, and filters that allows user 100 to perform the same analysis or comparisons over time.

### Infrastructure

Infrastructure supports how the system deploys/monitors services, ingests/stores data, load balances, and secures everything.

### Inquisitor

Inquisitor is a component that does active data collection by polling user 100 with customizable questions and surveys, recording their responses, and sending the data back to a central repository.

### Custom Inputs

In at least one embodiment, a feature may be provided to allow users 100 to create an endpoint for setups such as physical buttons or iOS shortcuts that record custom inputs into their datastore. User 100 may configure custom inputs with a unique URL to record custom datapoints as desired.

In at least one embodiment, a tag can be associated with more than one custom input. Every time the unique URL is accessed, each tag may be recorded as an individual metric datapoint. The custom input itself may also be recorded as an individual metric datapoint. iOS shortcuts and physical buttons may be configured to access the URL.

Recorded data may be viewed by adding desired metrics to user's 100 Timeline.

Functionality may be provided for creating, managing, and/or deleting custom inputs.

In at least one embodiment, user 100 is able to add values and units with their custom inputs.

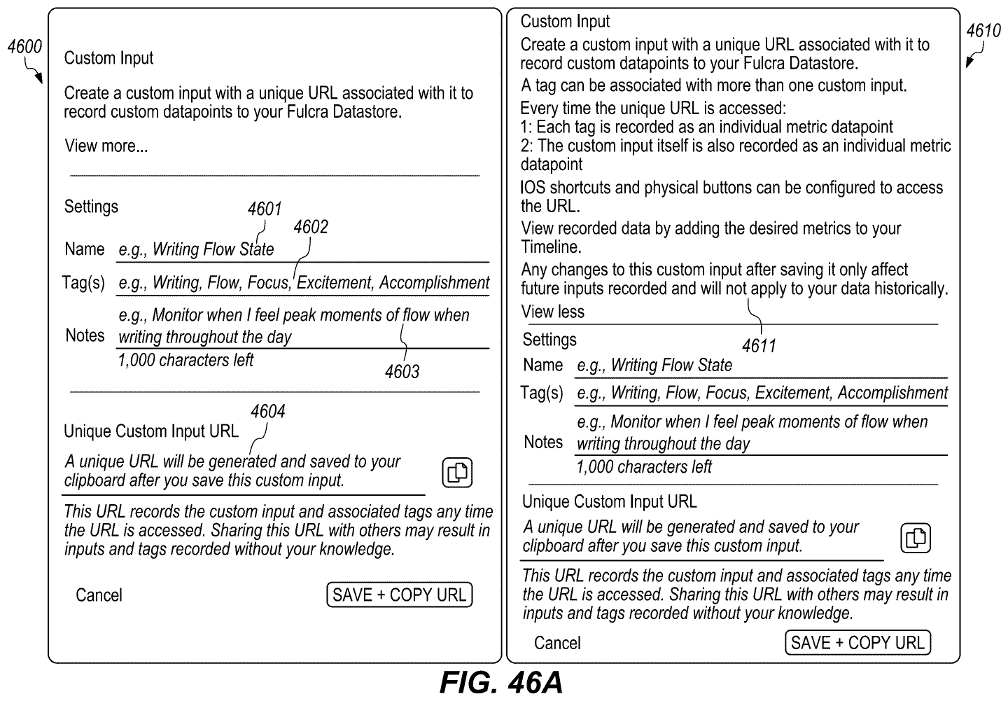

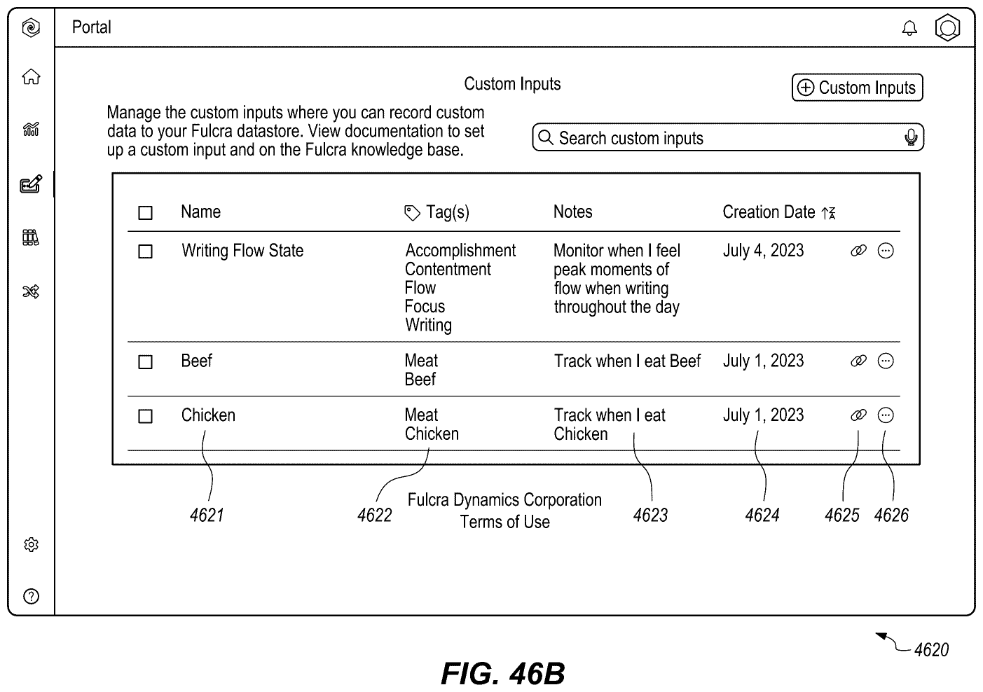

Referring now to FIGS. 46A and 46B, there are shown examples of screens 4600, 4610, 4620 for creating and managing custom inputs according to one embodiment.

FIG. 46A depicts an example of screen 4600 for creating a custom input. In name field 4601, user 100 can enter a name for the custom input configuration.

In tag field 4602, user 100 can type to search and view Recent tags. Upon selection of the tag field 4602, a dropdown may be presented to allow user 100 to scroll through to see recently selected tag values and a menu of potential moods and symptoms. If there are no matches, user 100 can still input any value into field 4602.

A custom input can contain one or more tags. If user 100 chooses to enter more than one tag, each of the tags will be recorded when the URL is accessed (e.g., one button press for “Writing Flow State” will record “true” or “1” for the tags “Contentment,” “Writing,” “Accomplishment,” “Flow,” and “Focus”).

Notes field 4603 allows user 100 to enter any notes as desired. A unique custom URL may be created in field 4604, which user 100 can then copy and paste as desired. Additional information 4611 may also be displayed.

In at least one embodiment, a tag can be associated with more than one custom input. In at least one embodiment, every time the unique URL is accessed:
1. Each tag is recorded as an individual metric datapoint; and
2. The custom input itself is also recorded as an individual metric datapoint.

iOS shortcuts and physical buttons can be configured to access the URL. User 100 can view recorded data by adding the desired metrics to their timeline 300. In at least one embodiment, any changes to this custom input after saving it only affect future inputs recorded and will not apply to user's 100 historical data.

Once user 100 has copied and pasted the custom URL, the custom input has been added. As shown in FIG. 46B, user 100 may be taken back to Manage Custom Inputs page 4620 where they can see their newly created custom input. Here, for each custom input, the following information may be presented: name 4621, tag(s) 4622, notes 4623, and creation date 4624. User 100 can click on link 4625 to view or copy the URL to the custom input. Action 4626 provides access to more actions that may be performed, such as editing the custom input, copying the URL, or deleting the custom input.

### Library

The library securely stores all of the configurations, contacts, databases, documents, histories, media, preferences, as well as workflows and processes that user 100 wants to make available for data analysis or access to other apps.

### Notebooks

A notebook is a collection of queries and filters using an internal notebook platform (such as one based on Jupyter) to create interactive snapshots of the data. A notebook is an internal tool that essentially serves as the “backend” of an Explorer view. It may include Python-coded, filtered, time-bound datasets with queries designed for the purpose of displaying results from particular experiment outcomes using the interactive visualizations that are optimized to the data being presented.

### Notifier

A component of the app that allows the system to:
inform user 100 that there are questions/polls that they can complete to provide the system with timely, contextual data;
nudge user 100 about something that needs to be done;
inform user 100 about a new insight; and/or
inform user 100 of other news related to the system or the app.

In at least one embodiment, user 100 is able to customize their notification settings.

### Portal

The Portal is a web-based application that allows users 100 to find and gain access to everything they need related to the system, all in one place. It is the home for accessing, for example:
Dashboard
   Timeline
   Explorer
   Reports, including Shareable Reports
Library
Documents
   Experiments Runbook
Integrations
Knowledge Base
Community

### Scheduler

A component of the app that allows the system to schedule questions/polls or reminder nudges for users 100 in order to collect timely, contextual data. In at least one embodiment, users 100 are able to schedule their own questions/polls or reminders.

### Shareable Reports

These are any team-created reports that user 100 wants to provision to someone on their personal team. This allows user 100 to share data for greater shared awareness while also limiting the scope of the data to which their team has access. In at least one embodiment, users 100 can create their own custom reports using the Timeline or Explorer that can be shared with others.

### Situation Reports

Static PDF reports that the team creates to regularly update users 100 on findings from experiments related to their goals so as to capture a snapshot of user's 100 progress in time. The data collected for these reports are designed in the Experiments Runbook. Users 100 can access these report files on their Dashboard under the Reports tab.

### ContextApp

In at least one embodiment, the system may include a mobile application referred to as a Context App that allows the system to collect passive data with DataPumphouse and active data with Inquisitor by scheduling questions/polls or reminders with Scheduler and notifying users 100 about the questions/polls with Notifier.

### Systems & Workflows

The team works with user 100 to design new and improved ways of working/living that can then be systematized and generalized for application across multiple users 100.

### This is You Reveal

An artistic exhibition of user's 100 data that represents the essence of who they are and highlights their accomplishments. The customization and physical scale is meant to inspire awe and feelings of being understood in user 100. In at least one embodiment, this display can be included in a weekly rollup that show's user's 100 data in an interactive weekly format.

### Timeline 300

A Dashboard tool that allows user 100 to view a time-based replay of their life in great detail so they can understand hidden trends and correlations across different areas of their life. Users 100 are then able to see a forecast of what is in store for them so that they can plan and make decisions accordingly. It is an alternative tool for looking at the same data that Explorer presents.

### User Management &Access Control

The layer that handles who can access what product components at which level of authority.

### Life API

In at least one embodiment, the system may also include a Life API, which may include one or more external-facing APIs. The Life API may enable one or more of the following:
securely grant another application access to query user data (such as for example, a hosted computational Jupyter notebook like Google Colab or Deepnote); security can be provided, for example, using OAuth2 workflows.
securely grant AI applications access to query user data (such as OpenAI Custom GPTs); in at least one embodiment, different OpenAPI specifications may be provided.
configure and access datashares;
configure and access custom inputs;
configure and access Inquisitor polls, Reflections, and/or the like;
access data writing code and publicly-available API client libraries;
use the provided backend to perform analytics on user data (such as by including descriptive statistics).

### System Architecture

According to various embodiments, the systems and methods described herein may be implemented on any electronic device or set of interconnected electronic devices, each equipped to receive, store, and present information. Each electronic device may be, for example, a server, desktop computer, laptop computer, smartphone, tablet computer, a router, a switch, and/or the like. As described herein, some devices used in connection with the systems and methods described herein are designated as client devices, which are generally operated by end users. Other devices are designated as servers, which generally conduct back-end operations and communicate with client devices (and/or with other servers) via a communications network such as the Internet. In at least one embodiment, the techniques described herein may be implemented in a cloud computing environment using techniques that are known to those of skill in the art.

In addition, one skilled in the art will recognize that the techniques described herein may be implemented in other contexts, and indeed in any suitable device, set of devices, or system capable of interfacing with existing enterprise data storage systems. Accordingly, the following description is intended to illustrate various embodiments by way of example, rather than to limit scope.

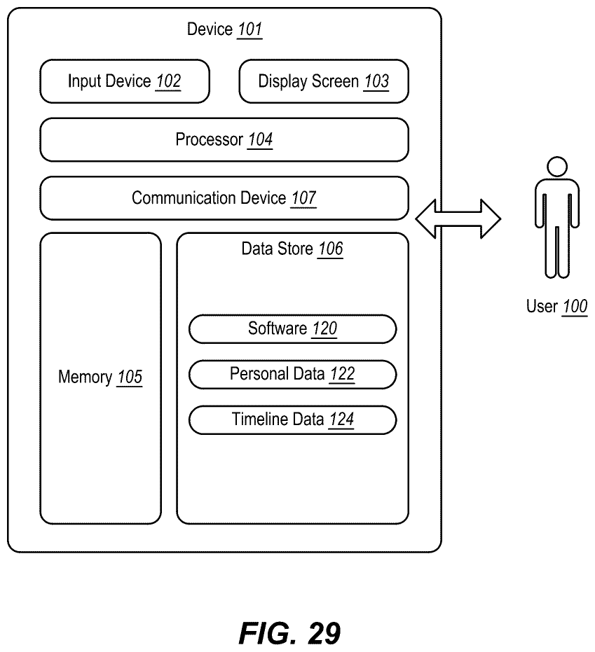

Referring now to FIG. 29, there is shown a block diagram depicting a hardware architecture for practicing the described system, according to one embodiment. Such architecture may be used, for example, for implementing the techniques of the system in a computer or other device 101. Device 101 may be any electronic device, and in some embodiments, may be a computer, smartphone, tablet, or wearable device that may be coupled to other devices via a wireless computing network.

In at least one embodiment, device 101 includes a number of hardware components that are well known to those skilled in the art. Input device 102 may be any element that receives input from user 100, including, for example, a keyboard, mouse, stylus, touch-sensitive screen (touchscreen), touchpad, trackball, accelerometer, microphone, or the like. Input may be provided via any suitable mode, including for example, one or more of: pointing, tapping, typing, dragging, and/or speech. In at least one embodiment, input device 102 may be omitted or functionally combined with one or more other components.

Data store 106 may be any magnetic, optical, or electronic storage device for data in digital form; examples include flash memory, magnetic hard drive, CD-ROM, DVD-ROM, or the like. In at least one embodiment, data store 106 stores information that can be utilized and/or displayed according to the techniques described below. Data store 106 may be implemented in a database or using any other suitable arrangement. In another embodiment, data store 106 may be stored elsewhere, and data from data store 106 may be retrieved by device 101 when needed for processing and/or presentation to user 100. Data store 106 may store one or more data sets, which may be used for a variety of purposes and may include a wide variety of files, metadata, and/or other data.

In at least one embodiment, data store 106 may store data such as software 120, which may include firmware, BIOS, a boot loader, an operating system, and/or the like. Data store 106 may further store personal data 122, which may describe user 100, and/or timeline data 125, which may be collected using the techniques described herein for presenting a unified timeline to user 100. In at least one embodiment, such data may be stored at another location, remote from device 101, and device 101 may access such data over a network, via any suitable communications protocol.

In at least one embodiment, data store 106 may be organized in a file system, using well known storage architectures and data structures, such as relational databases. Examples include Oracle, MySQL, and PostgreSQL. Appropriate indexing may be provided to associate data elements in data store 106 with each other. In at least one embodiment, data store 106 may be implemented using cloud-based storage architectures such as Google Cloud Platform (available from Google Inc. of Mountain View, California), NetApp (available from NetApp, Inc. of Sunnyvale, California), and/or AWS (available from Amazon Web Services, Inc. of Seattle, Washington).

Data store 106 may be local or remote with respect to the other components of device 101. In at least one embodiment, device 101 is configured to retrieve data from a remote data storage device when needed. Such communication between device 101 and other components can take place wirelessly, by Ethernet connection, via a computing network such as the Internet, via a cellular network, or by any other appropriate communication systems.

In at least one embodiment, data store 106 may be detachable in the form of a CD-ROM, DVD, flash drive, USB hard drive, or the like. Information may be entered from a source outside of device 101 into data store 106 that is detachable, and later displayed after data store 106 is connected to device 101. In another embodiment, data store 106 may be fixed within device 101, or may be enabled using cloud-based storage techniques.

In at least one embodiment, data store 106 may be organized into one or more well-ordered data sets, with one or more data entries in each set. Data store 106, however, can have any suitable structure. Accordingly, the particular organization of data store 106 need not resemble the form in which information from data store 106 is displayed to user 100 on display screen 103. In at least one embodiment, an identifying label is also stored along with each data entry, to be displayed along with each data entry.

Display screen 103 may be any element that displays information such as text and/or graphical elements. In particular, display screen 103 may present a user interface for entering, viewing, configuring, selecting, editing, downloading, and/or otherwise interacting with datasets as described herein. In at least one embodiment, as described herein, display screen 103 may display an interactive, unified timeline for presentation of events relevant to user 100.

In at least one embodiment where only some of the desired output is presented at a time, a dynamic control, such as a scrolling mechanism, may be available via input device 102 to change which information is currently displayed, and/or to alter the manner in which the information is displayed. In at least one embodiment, display screen 103 can be omitted or functionally combined with one or more other components.

Processor 104 may be a conventional microprocessor for performing operations on data under the direction of software, according to well-known techniques. Memory 105 may be random-access memory, having a structure and architecture as are known in the art, for use by processor 104 in the course of running software.

Communication device 107 may communicate with other computing devices through the use of any known wired and/or wireless protocol(s). For example, communication device 107 may be a network interface card (“NIC”) capable of Ethernet communications and/or a wireless networking card capable of communicating wirelessly over any of the 802.11 standards. Communication device 107 may be capable of transmitting and/or receiving signals to transfer data and/or initiate various processes within and/or outside device 101.

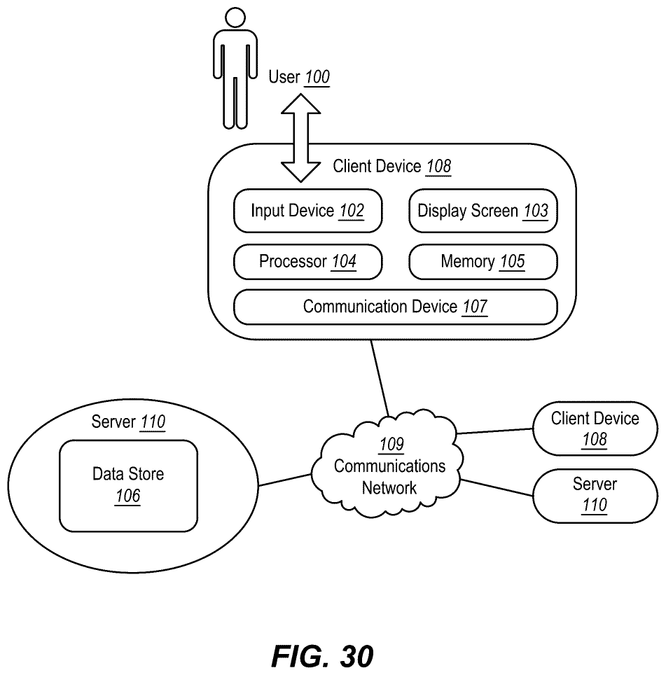

Referring now to FIG. 30, there is shown a block diagram depicting a hardware architecture in a client/server environment, according to one embodiment. Such an implementation may use a “black box” approach, whereby data storage and processing are done completely independently from user input/output. An example of such a client/server environment is a web-based implementation, wherein client device 108 runs a browser that provides a user interface for interacting with web pages and/or other web-based resources from server no. Items from data store 106 may be presented as part of such web pages and/or other web-based resources, using known protocols and languages such as Hypertext Markup Language (HTML), Java, JavaScript, and the like.

Client device 108 may be any electronic device incorporating input device 102 and/or display screen 103, such as a desktop computer, laptop computer, personal digital assistant (PDA), cellular telephone, smartphone, music player, handheld computer, tablet computer, kiosk, game system, wearable device, or the like. Any suitable type of communications network 109, such as the Internet, may be used as the mechanism for transmitting data between client device 108 and server no, according to any suitable protocols and techniques. In addition to the Internet, other examples include cellular telephone networks, EDGE, 3G, 4G, 5G, long term evolution (LTE), Session Initiation Protocol (SIP), Short Message Peer-to-Peer protocol (SMPP), SS7, Wi-Fi, Bluetooth, ZigBee, Hypertext Transfer Protocol (HTTP), Hypertext Transfer Protocol Secure (HTTPS), Transmission Control Protocol/Internet Protocol (TCP/IP), and/or the like, and/or any combination thereof. In at least one embodiment, client device 108 transmits requests for data via communications network 109, and receives responses from server 110 containing the requested data. Such requests may be sent via HTTP as remote procedure calls or the like.

In one implementation, server 110 is responsible for data storage and processing, and incorporates data store 106. Server no may include additional components as needed for retrieving data from data store 106 in response to requests from client device 108.

As described above in connection with FIG. 29, data store 106 may be organized into one or more well-ordered data sets, with one or more data entries in each set. Data store 106, however, may have any suitable structure, and may store data according to any organization system known in the information storage arts, such as databases and other suitable data storage structures. As in FIG. 29, data store 106 may store data, including but not limited to software 120, personal data 122, timeline data 125, and/or the like; alternatively, such data may be stored elsewhere (such as at another server) and retrieved as needed.

In addition to or in the alternative to the foregoing, data may also be stored in data store 106 that is part of client device 108. In some embodiments, such data may include elements distributed between server 110 and client device 108 and/or other computing devices in order to facilitate secure and/or effective communication between these computing devices.

As discussed above in connection with FIG. 29, display screen 103 may be any element that displays information such as text and/or graphical elements. Various user interface elements, dynamic controls, and/or the like may be used in connection with display screen 103.

As discussed above in connection with FIG. 29, processor 104 may be a conventional microprocessor for use in an electronic device to perform operations on data under the direction of software, according to well-known techniques. Memory 105 may be random-access memory, having a structure and architecture as are known in the art, for use by processor 104 in the course of running software. Communication device 107 may communicate with other computing devices through the use of any known wired and/or wireless protocol(s), as discussed above in connection with FIG. 29.

In one embodiment, some or all of the system may be implemented as software written in any suitable computer programming language, whether in a standalone or client/server architecture. Alternatively, some or all of the system may be implemented and/or embedded in hardware.

Notably, multiple client devices 108 and/or multiple servers no may be networked together, and each may have a structure similar to those of client device 108 and server 110 that are illustrated in FIG. 30. The data structures and/or computing instructions used in the performance of methods described herein may be distributed among any number of client devices 108 and/or servers 110. As used herein, “system” may refer to any of the components, or any collection of components, from FIGS. 29 and/or 30, and may include additional components not specifically described in connection with FIGS. 29 and 30.

In some embodiments, data within data store 106 may be distributed among multiple physical servers. Thus, data store 106 may represent one or more physical storage locations, which may communicate with each other via the communications network and/or one or more other networks (not shown). In addition, server 110 as depicted in FIG. 30 may represent one or more physical servers, which may communicate with each other via communications network 109 and/or one or more other networks (not shown). Part of data store 106 may reside on device 101 and/or client device 108.

In one embodiment, some or all components of the system may be implemented in software written in any suitable computer programming language, whether in a standalone or client/server architecture. Alternatively, some or all components may be implemented and/or embedded in hardware.

### Conceptual Architecture

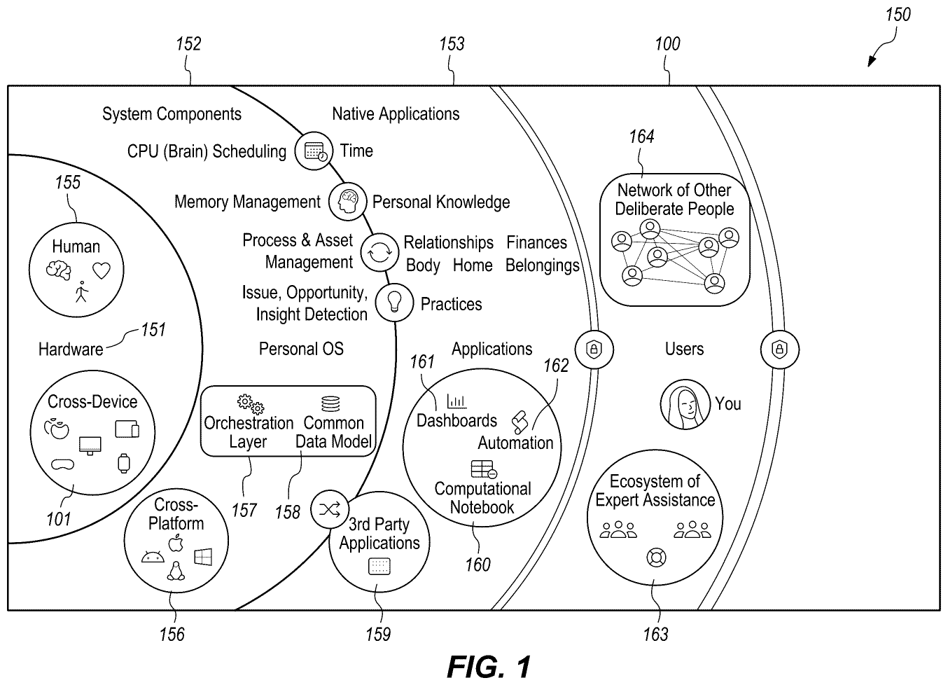

Referring now to FIG. 1, there is shown a block diagram depicting a conceptual architecture 150 for the described system, according to one embodiment.

The system may include hardware 151, a personal operating system (OS) 152, various applications 152, and users 100. Hardware 151 may be configured to operate across multiple devices 101, some or all of which may be configured to receive input and information from humans 155 in any suitable way, including by active or passive means (e.g., by directed input or by measuring activity, heartbeat, and/or the like). Humans 155 may include users 100, but they may also include non-users of the system. In at least one embodiment, a Life API may be used in lieu of personal OS 152.

Personal OS 152 may include an orchestration layer 157 and a common data model 158. In at least one embodiment, Personal OS 152 may operate using a cross-platform model 156 to enable improved flexibility. Personal OS 152 may include various components directed to various functions, such as for example: CPU (brain) scheduling; memory management; process and asset management; and/or issue, opportunity, and insight detection. Further details concerning Personal OS 152 are provided herein.

Applications 153 may include, for example, dashboards 161, automation 162, computational notebooks 160, and/or third-party applications 159. Some components may be native applications configured to support the various components of Personal 152, including, for example, a time-based application to support CPU (brain) scheduling; a personal knowledge application to support memory management; applications directed to relationships, finances, body, home, and belongings, to support process and asset management; and/or a practices application to support issue, opportunity, and insight detection.

Users 100 include, in addition to the primary user 100 who may be interacting with the system, network 164 of other people who may interact with the primary user 100 and provide input that may be useful to generating or maintaining the universal timeline. In addition, in at least one embodiment, an ecosystem 163 of expert assistance may also be provided.

### Personal Operating System 152

In at least one embodiment, the described system implements a personal operating system (OS) 152 for amplifying human potential. The system may automatically correlate relevant digital data about user's 100 life, instrumenting automation where possible, and augmenting user's 100 awareness with expertise where they need it.

In at least one embodiment, the system may perform the following functions which enable it to act as a personal operating system:
Create an electronic map of user's 100 life, which may be presented in an interactive timeline display as described in more detail herein.
Perform a Gap Analysis against current life capabilities and supporting team.
Develop digital “runbooks” that define repeatable processes for user 100, their team, and the underlying technology.
Establish user's 100 Personal Board of Directors, which may include experts in domains ranging from time to health to finance and research.
Continually measure and improve user's 100 life processes to help them achieve peak flow in each category, documenting their evolution over time.
Deliver unique insights about user's 100 life and data-driven recommendations for habits to install.

In at least one embodiment, Agents may be looped in to further enhance data collection, processing, and display functions.

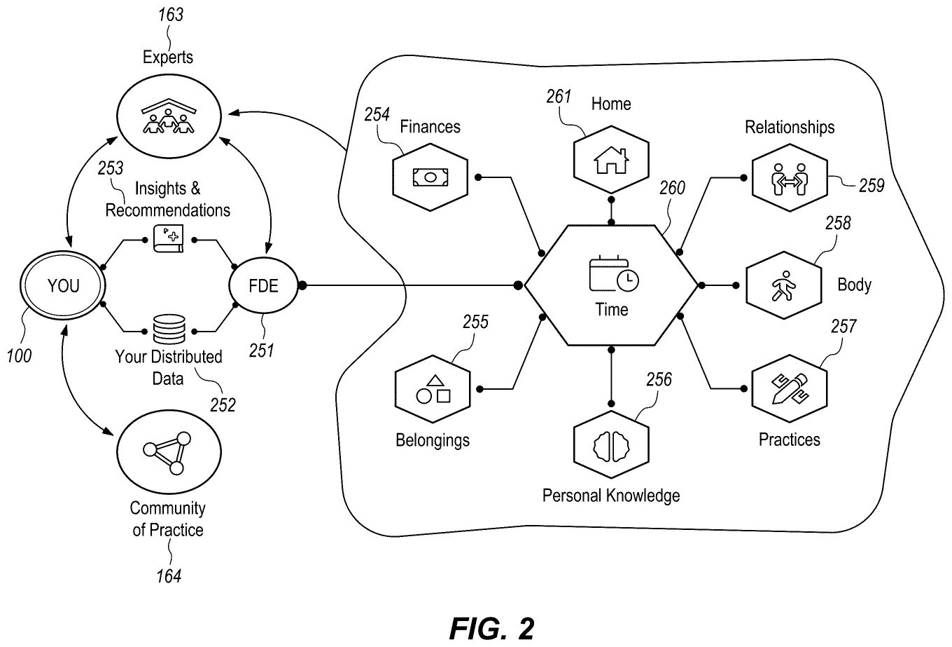

FIG. 2 is a block diagram depicting an example of the integration of various components in an embodiment of the system.

In at least one embodiment, the system provides a Context Layer, which allows context to be added to artificial intelligence (AI) agents and/or other apps, as described in more detail herein. The system may be implemented as a platform that enables people to see the big picture, while also drilling in to optimize every aspect of their lives.

In at least one embodiment, the system described herein pairs advanced analysis of user's 100 most important data with world-class domain expertise in the form of experts 163, as well as a team of Forward Deployed Engineers (FDEs) 251 to empower user 100 and their team to understand the outcomes of their actions and to make highly informed decisions that are based on real data from user's 100 life. However, the use of FDEs 251 is optional and can be omitted.

Once user's 100 life is instrumented, the system continually measures and improves user's 100 life processes to help them achieve peak flow.

For example, the system can enable user 100 to access their data through computational notebooks at any time. Such access may be provided via a Life API. Data science and FDEs 251 can apply analytics to supplement user's 100 information. Those insights then support user's 100 work with FDEs 251 and/or other experts 163 to apply new habits or tweak practices that align with user's 100 goals.

Accordingly, as depicted in FIG. 2, data can be provided to user 100 from community of practice (which may include network 164 of other people who may interact with the primary user 100), as well as experts 163 and FDEs 251. Data can take the form of insights and recommendations 253 to inform user 100 of concrete steps they can take to improve their life and address issues. User 100 can also access and contribute to a set of distributed data 252 that may be stored in various data stores, whether locally or in the cloud.

Data that is provided to FDEs 251, experts 163, and/or user 100, may be organized based on time 260, for example, in an interactive timeline display as described herein. Data for the interactive timeline display may come from any of a number of sources, including for example: data about finances 254, data about user's 100 home 261, data about relationships 259, data about user's 100 body 258, data about user's 100 practices (including habits and/or activity) 257, personal knowledge 256, and/or belongings 255. As described, all such data can be unified, processed, and normalized for presentation in the interactive timeline display.

### Conduct Lifestyle Experiments

In at least one embodiment, the system can also allow user 100 to work with engineering and coaches to conduct various lifestyle or habit experiments. While utilizing personalized data analytics, user 100 may add or subtract from their life intentionally and have the means to track the outcome from their changes. In addition, as described herein, users 100 can share some or all of the data with other users 100, either in static or dynamic form.

### Deploy a Digital Second Brain

While user 100 is working with FDEs 251 or with AI models, supporting documentation and data analysis may be automatically and/or manually conducted in preparation for user 100. The system may securely organize user's 100 lifestyle documentation post-analytics, and may provide user 100 with access to their personal insights all in one place. Collections of documentation for user's 100 data can be reviewed to understand how different domains of their life interact with one another.

### Activate the Best Help

A team of world class specialists, or experts 163, in a variety of domains can be made available and can provide input to the system. User 100 can work with them to help optimize their life backed by their data and supported by best-in-class coaches. Bring user's 100 own talent

In at least one embodiment, user 100 can have their personal coach or specialist apply to work with their data, via a shared awareness platform. The coach can be provided with access to user's 100 data and the system's analytics. The system may then operate as tooling for them to support user's 100 coaching goals. User 100 can thereby work with an expert that already knows their goals and has a proven track record of collaborating well with them.

### Functional Components

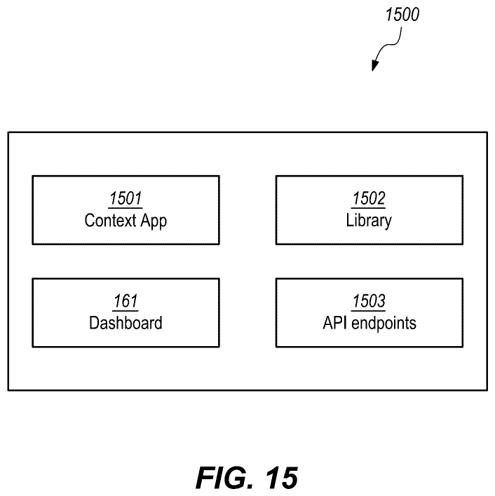

Referring now to FIG. 15, there is shown a block diagram depicting a functional architecture 1500 for implementing the described system, according to one embodiment.

In at least one embodiment, the system may include the following components, which may be communicatively coupled with one another to implement the functionality described herein:
Context App 1501 or browser plug-in running on users' devices 101 (such as, for example, their phones) may allow users 100 to collect the data they are generating on their devices through data aggregation services and apps such as HealthKit, Google Fit, Android Health Connect, calendar applications and services, location apps and services, and other phone and app data analytics. Context App 1501 can also serve as a research tool for querying user 100 at just the right times so they can enrich their passive data with context. These product capabilities allow the system to become a serious engine for collecting data that may otherwise be difficult to collect.
Dashboard 161 may serve as the main command center where users 100 can see their data visualized in different ways. In at least one embodiment, dashboard 161 may present an interactive timeline display that allows user 100 to replay their life in detail. In at least one embodiment, dashboard 161 may present an explorer display that allows user 100 to dig into interesting correlations to gain insights that they can share with other people, especially the experts they are asking for help. These product capabilities allow the system to become a centralized source of ubiquitous shared awareness. Additional details concerning these displays are provided herein.
Library 1502 may securely store all of the data collected via context app 1501, as well as documents created for user 100, in one location. The system may automatically de-duplicate and normalize the data into structured, machine readable formats, making it more suitable for cryo (long-term) storage, combining with third party data, and/or processing by third party solutions. These product capabilities allow the system to become a trusted preservation and orchestration layer with a common data model.
In at least one embodiment, Life API 1503 may be created to make specific streams of user data available to third party apps and/or AI tools. This streaming data export service can help enhance users' 100 experience when using those platforms. These product capabilities allow the system to become the fabric into which other creators can weave their tools.

### Platform

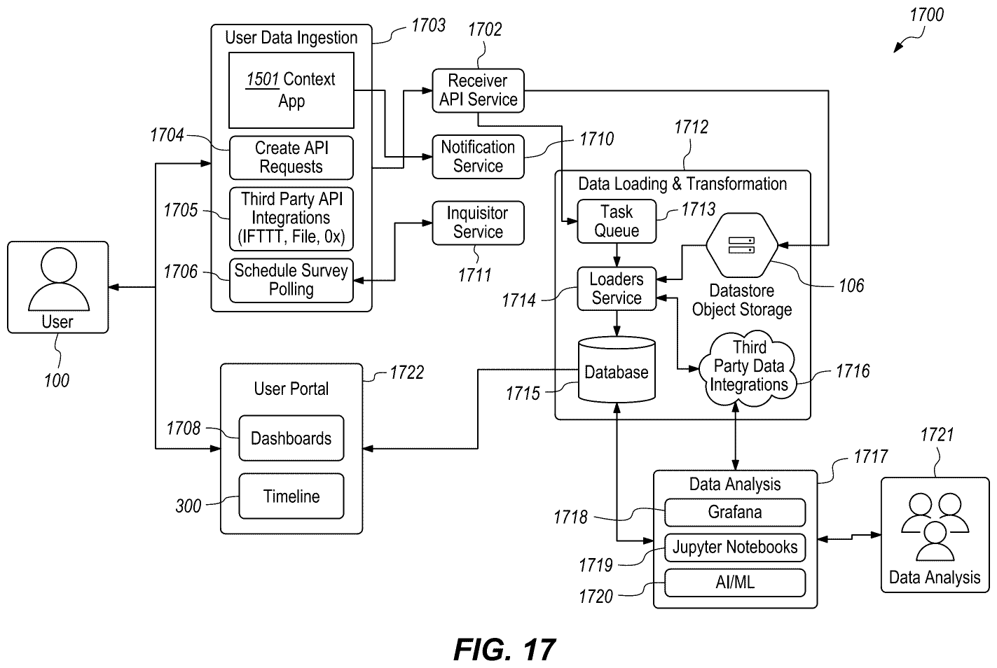

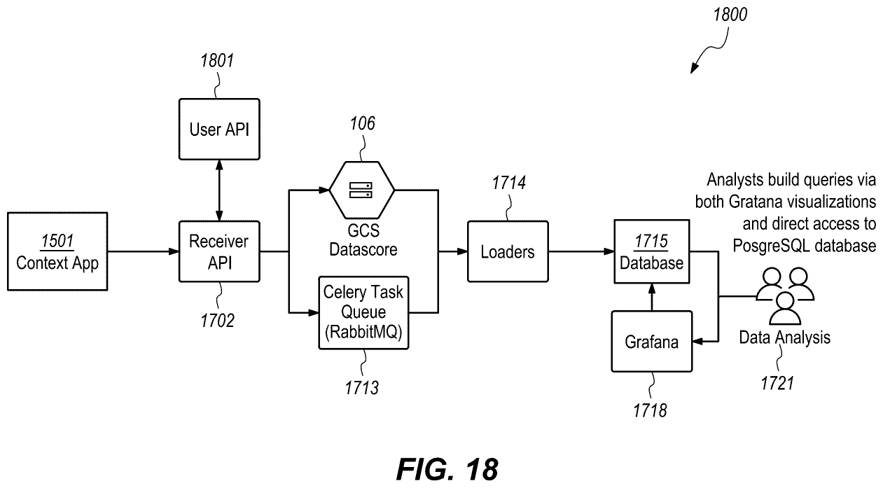

Referring now to FIG. 17, there is shown a block diagram depicting an example of a platform 1700 for implementing the described system, according to one embodiment. Referring now to FIG. 18, there is shown a block diagram depicting an example of a data flow 1800.

The following components may be included:
User Data Ingestion module 1703 may include Context App 1501, which is an iOS application responsible for collecting client data (for example via Apple's APIs or the like) and sending it to a central repository. In at least one embodiment, Context App 1501 collects and ships user telemetry from sources such as Apple HealthKit and/or other sources, and sends it to Receiver API 1702. User Data Ingestion module 1703 may also include functionality for providing Direct API Requests 1704, Third Party API Integrations 1705, and Stochastic Survey Pulling 1706.
Receiver API 1702 may be an HTTP API service responsible for ingesting data from client sources, primarily Context. Data sent to Receiver API 1702 may be kept for long term storage in a data store (currently Google Cloud Storage) for later processing. Third party services may be integrated with Receiver API 1702 to provide additional sources of user data. In at least one embodiment, Receiver API 1702 may issue tasks to Task Queue 1713 to process user source data.
User Portal 1722 provides mechanisms by which user 100 can interact with the system, including dashboards 1708 and timeline 300 (described in more detail herein).
User API Service 1801 is the canonical source of truth for clients of the system. Each client that authenticates with the platform may be provided with a unique identifier that is used within the platform. This service may also be responsible for the client's user preferences, group relationships between clients, and connected social networks. In at least one embodiment, Receiver API 1702 may use User API 1801 for user authorization. In at least one embodiment, other internal APIs may communicate with User API Service 1801 to obtain information about users, and to configure elements such as datashares, settings, and/or the like.
Notification service 1710 may be responsible for sending notices to a client's iOS device 101.
Inquisitor service 1711 may provide services for: sending polls that were configured by user 100 itself; interacting with third-party services to send notification methods such as Apple's APNs, Google's Firebase Cloud Messaging, and/or SMS providers such as Twilio; algorithmically deciding when to send follow-up polls; and/or deciding which text(s) to use in a poll.
Data Loading and Transformation Module 1712 includes the following subcomponents:
   Data store 106: A cloud object storage service such as Google Cloud Storage may serve as a long term data store of client data. All data may be sorted by User ID & data type and may remain unmodified from how it was received. In at least one embodiment, collected user data may be stored in object storage and encrypted with a key unique to each user. Users 100 manage the lifetime of their encryption keys and can revoke access to their data at any time, as well as access granted by user 100 to any third party tools, apps, or services. In at least one embodiment, source data may be kept in data store 106 depending on the ingestion method. Batch uploads may have their source data removed after being processed.
   Loaders 1714 may be implemented as Celery task workers responsible for retrieving client data from data store 106, processing it, and processing and transforming the received source data into SQL tables for storage in database 1715.
   Database 1715: In at least one embodiment, data processed by Loaders 1714 may be inserted into tables of database 1715, which may be implemented as a PostgreSQL database or the like.
   Task Queue 1713 may provide task management, and may send tasks to an available loader 1714 worker. In at least one embodiment, Task Queue 1713 may be implemented as a Celery queue or hosted via a cloud system like Google Cloud Pub/Sub.
   Third Party Integrations 1716 may be queried to supplement data.
Data Analysis Tools 1717: Tools such as Grafana 1718, Jupyter Notebooks 1719, and Bokeh 1720 may be used to retrieve processed data from the database for analysis as well as building visualizations. Input from data analysts 1721 may be used by tools 1717; for example, analysts 1721 may build queries via Grafana visualizations and/or direct access to database 1715.

### Overall Architecture

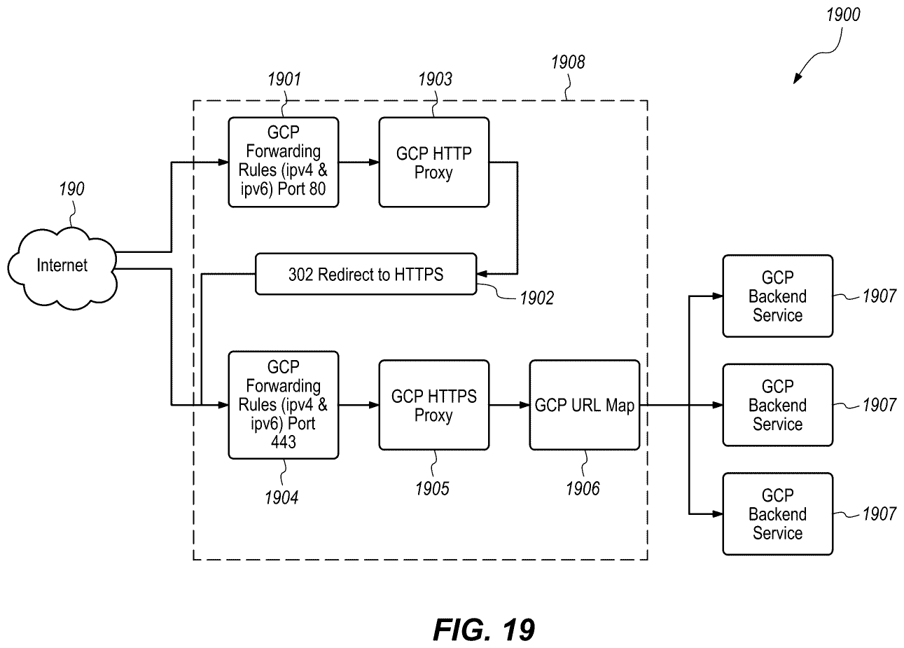

Referring now to FIG. 19, there is shown a block diagram depicting an example of an overall architecture 1900 for implementing the described system according to one embodiment. In at least one embodiment, back-end services 1907 may be fronted by load balancer 1908, such as a Google Cloud Platform (GCP) load balancer.

TLS certificates for HTTPS services may be terminated at the GCP load balancer and automatically managed, for example by Google. In order for Google Managed Certificates to properly provision, the certificate may be attached to HTTPS Target Proxy 1903, and DNS records for the certificate hostname may point to the GCP load balancer.

Services like CDN caching, IAP authentication, and the like may be configured at GCP Backend Service 1907 for individual services. A Backend Service may represent multiple types of GCP resources, including VM Instance Groups, Cloud Run Serverless Services, and/or GCP Storage Buckets.

In at least one embodiment, load balancer 1908 may include various components to implement elements depicted in connection with FIG. 17. Such components may include, for example, GCP forwarding rules 1901 and 1904, GCP HTTP Proxy 1903, GCP HTTPS Proxy 1905, and GCP URL map 1906. Redirection component 1902 may also be provided, to redirect traffic from 1903 to 1904.

### Digital Product Information Architecture

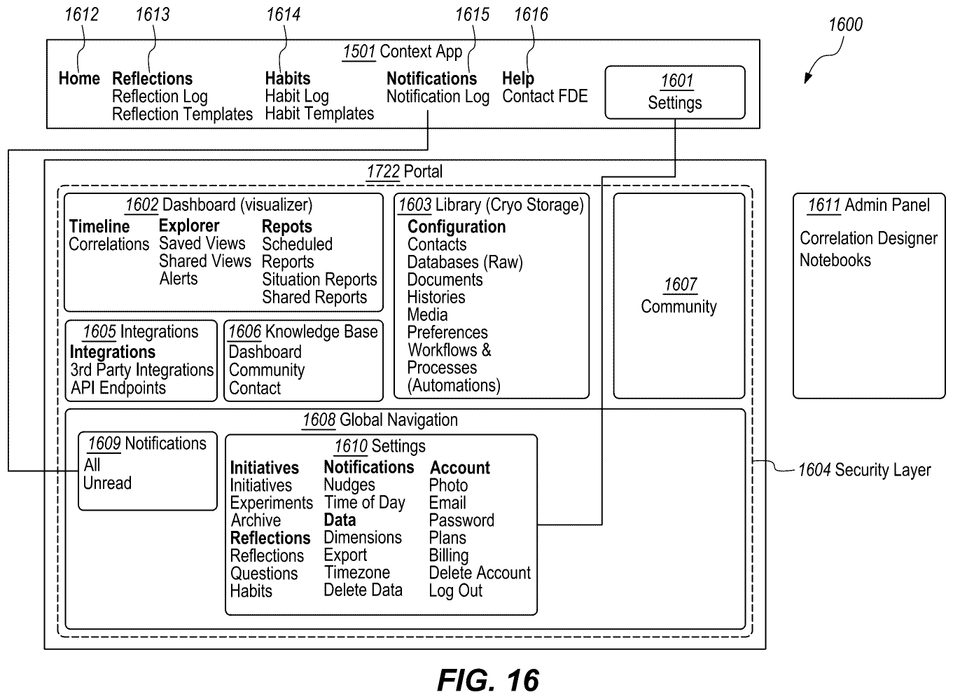

Referring now to FIG. 16, there is shown an example of a cohesive information architecture 1600 for the described system that enables the user experience to be smooth and seamless.

In at least one embodiment, there are two main tools that user 100 can access: Context App 1501 and Portal 1722. Admin panel 1611 is an internal tool that is used to customize the experience for users 100.

### ContextApp 1501

In at least one embodiment, Context App 1501 may include the following sections:
Home 1612: This is the place where users 100 can see quick visualizations of the data they've collected.
Reflections 1613 are a set of questions that are triggered either stochastically or on a fixed schedule. The data represents what was in user's 100 minds at the time of the responses. This section allows user 100 to access their incomplete reflections, a log of all the reflections that have been triggered with their progress, and templates of reflections they can try out for themselves.
Habits 1614 are intentions for action towards goals that are triggered either stochastically or on a fixed schedule. The data represents what actions the person took in alignment with their goals. This section allows user 100 to access their incomplete habits, a log of all the habits that have been triggered with their progress, and templates of habits they can try out for themselves.
Notifications 1615 let user 100 know when they have a reflection or habit to complete. This section allows user 100 to access their unread/read notifications and a log of all the notifications that they have received.
Help 1616: This section allows user 100 to contact their team of Forward Deployed Engineers (FDEs) 251 via any suitable communication means such as Intercom, and/or to access the Knowledge Base.

In at least one embodiment, the system does not require user 100 to manually indicate whether habits 1615 are complete or incomplete. Rather, the system may use the data generated by the apps that user 100 already uses to update the habit status. Most “good” habits may already be tracked within the apps people use. For example, if user 100 wants to track meditation, yoga, and/or sleep by 9:30 pm habits, several tools are available. For good habits that need to be tracked manually like “reset kitchen after dinner,” the system may turn those into button presses or quick polls. Bad habits, in general, often require manual input and thus harder to track. For example, “quit smoking” requires the person to report when they have done it. As a result, typically: they either 1) benefit from the act of recording it which reinforces not doing it, or 2) do not record it at all. There are more apps aimed toward good habit formation than specifically breaking certain bad habits. In at least one embodiment, the system can find ways of tracking things like “avoid sugary foods” by monitoring blood glucose levels or through some other data stream and ask user 100 to confirm, rather than requiring user 100 to manually record such information.

### Portal 1722

In at least one embodiment, Portal 1722 may include the following sections:
Dashboard 1602: This section allows users 100 to access, understand, manipulate, and share their data. In at least one embodiment, it may include the following subsections:
   Timeline 300 provides a unified, normalized, interactive view along a time scale. It allows users 100 to replay their data as it happened so they can understand trends across time. They can also share snapshots of their life with other people.
   Explorer allows users 100 to manipulate their data so they can understand correlations across different areas of their life. They can also share the interesting correlations they find with other people.
   Reports allows users 100 to access their reports. Users 100 can create custom-scheduled reports of data segments they are interested in tracking on a specific cadence that they want to share out to the experts they are working with. Situation Reports created by the team may be saved here for easy access. Any of the reports that users 100 make available externally to other people may be tracked here.
Library 1603: This section allows users 100 to access important information they want to preserve long-term (i.e., in “cryo” storage) in one place. While nascent machine learning and artificial intelligence technologies begin to advance over time, users 100 are able to bring their diverse and intensive longitudinal datasets collected then processed through the system for those technologies to draw upon, setting them far ahead of the curve to extract the most useful outputs. In at least one embodiment, library 1603 may include subsections for the following functionality:
   User 100 can save digital configurations to make it easy to port over to new tools or share with others to get on the same page. Examples include a mechanical keyboard layout and macro configuration.
   User 100 can have a unified and de-duplicated list of their contacts that are relatable to other data in their library.
   User 100 can upload and preserve important datasets that they want to incorporate into their data corpus. One example is a database of all the books they read with notes.
   User 100 can upload important documents that they want to preserve as digital representations of themselves. An example includes a personal mission statement with north stars for the year. Here they can also find documents (e.g., Experiments Runbook) created for them by the team.
   User 100 can access or upload the histories that they generate in other applications so that they can become available for analysis. An example includes browser history so they can remember what they read, were looking for, or thinking.
   Inventory: User 100 can save logs of their assets (both physical and digital) and where to access them. Examples include heirlooms to be passed down, keys, accounts, passwords, and/or the like.
   Media: User 100 can upload their photos, videos, and/or music via the app, so they can remember what they were doing and interested in at the time they were taken/listened to.
   User 100 can save their preferences that are not as easily captured in a configuration file.
   Workflows and Processes (Automation): User 100 can save runbooks for how they have streamlined their work. In at least one embodiment, these may include automations created to make their workflows and processes smoother for them.
Integrations 1605: This section may include third-party integrations that allow user 100 to view the apps that are sending data to the system, and to manage how the data is stored in the library and displayed on apps. In at least one embodiment, user 100 can create and manage Life API 1503 so that they can send streams of the data normalized by the platform to other apps, including AI tools.
Knowledge Base 1606: This section may provide users 100 with a centralized place to learn the features of the system's tools and troubleshoot on their own before contacting their team.
Community 1607: This section may provide users 100 with access to each other and provides ideas about how to make the most of their toolset.
GlobalNavigation 1608: This section may be accessible from anywhere in Portal 1722 and may give users 100 access to important Notifications 1609, Settings 1610, and Account Preferences.

### Admin Panel

In at least one embodiment, Admin Panel 1611 may be provided, as an internal-only interface that allows team members to configure and customize the product suite to user's 100 needs. Team members can also access internal tools such as the Correlation Designer and Notebooks.

### Services

### Data Science Driven Decisions

In at least one embodiment, a custom measurement, reporting and experimentation stack can be built for user 100, and user 100 can employ such a stack to make better decisions, either manually or as part of the platform provided by the described system. The system can help user 100 identify the questions they want answered, design the n=1 experiments that will help them make deliberate changes to their routines and way of life, collect data, analyze outcomes, provide feedback, and provide accountability. By working with data that user 100 is already generating, a custom life analytics and self-actualization stack for user 100 can be built.

### Delegation Engine for Productivity

In at least one embodiment, the system allows user 100 to manage their time based on outcomes rather than availability, and to reduce the cognitive load of getting help. Sometimes figuring out what to delegate and how to assign it may be even more difficult than figuring out whom to delegate to. The system provides tools to allow the use of user's 100 times to be holistically understood, so that low-value tasks can be identified and turned into replicable and delegatable activities; they can then be completed by the system itself, or their delegation can be managed so that outcomes can be reported back to user 100. The system can do the work to help user 100 figure out how to free themselves from the things that often seem easier to do themselves rather than offload, helping user 100 focus more of their time on the things that only they can do in a repeatable manner, integrating all of the results into a data-driven model.

### Financial Situational Awareness

In at least one embodiment, the system can aggregate and centralize all of user's 100 financial data, and then build a personal chart of accounts and unified reporting across user's 100 personal conglomerate. The system can thus provide functionality that goes beyond bookkeeping, but can provide situational awareness, cash flow management and modeling, and understanding where user 100 is under-resourced, so as to enable user 100 to achieve the best possible outcomes. Using the principles of data science, the system can explore multiple ways to increase user's 100 overall health, well-being, happiness, and/or wealth, identifying how a change in one habit might impact other areas of user's 100 life.

In at least one embodiment, for example, the system is able to ingest order history from online retailers.

### Train Intuition

In at least one embodiment, the system allows user 100 to privately document their predictions on any timescale and track them against outcomes. The resultant feedback loop can enable user's 100 intuitions to improve. By capturing user's 100 predictions about the future, the system can create the models and the scorecard to understand the performance of user's 100 intuition.

### People

### Personal Board of Directors

In at least one embodiment, a personal board of directors can be established and managed for user 100, by identifying and scheduling the right expertise in a given area when user 100 needs it, providing the board with the right information to be able to equip user 100 with real insight. This can be done as one-off projects, as regular “personal board of directors meetings,” or anything in-between.

### Empower Your Time Guardian

In at least one embodiment, the system allows user 100 to install an intelligent, learning filter between distractions and user's 100 attention. The system can be used as a gatekeeper to help keep low-value meetings off user's 100 schedule and keep pace. The system can collaborate with user 100 to figure out parameters for such time management tasks, to understand when exceptions should be surfaced to user 100, and to identify what is truly worth user's 100 time.

In this manner, the system can become a digital chief of staff to help user 100 manage their time and attention, with data-driven feedback loops to constantly improve the quality of the decisions that determine how user 100 uses their time. The system can integrate all of these activities and outcomes into user's 100 personalized data model that allows user 100 to repeat what is successful on an ongoing basis.

As described in more detail herein, the system may provide functionality allowing users 100 to selectively share data with other users 100, either on a static or dynamic basis.

### Cybernetically Enabled Team

As the system adds observability for each area that is important to user 100, the system can build out a map of all the areas of responsibility in user's 100 life. In tandem, the system can build out a map of user's 100 support team: everyone who works full-time for user 100, as well as the consultants and professionals user 100 brings to bear to solve particular problems. The system can draw any existing gaps into focus, and then build out the information systems user 100 needs to activate their staff to match accountability to responsibility.

Many users 100 may need experts in accounting, law, schedule management, and a host of other disciplines that define an individual life. In at least one embodiment, the system can analyze the capabilities of user's 100 current team of experts and help user 100 make sure they have the best-in-class advisors and problem-solvers assigned to every core area. The system can aggregate and correlate the reporting user 100 is receiving from all their experts into a centralized report, which, over time, can become a series of repeatable, visual analytics in user's 100 personal interface.

### Life Ops

### Personal Research and Experiment Design

In at least one embodiment, the system can help user 100 find answers as well as ask the right questions. The system can also help user 100 know and find the things that are most important to them with minimal involvement and distraction. Learning and research tasks can be offloaded into another person's brain, and synced back up when information becomes knowledge. Knowledge may be turned into repeatable operations. For complex problems with no general solution (for instance, diet choices), the system can find what works for user 100 with rigorous n=1 experiment design, data collection, and analysis.

### Secure Cybernetic Living

In at least one embodiment, the system can help user 100 prepare in advance for a disaster or security incident. User's 100 defenses can be matched to their assets and risks. By implementing the system's approach to secure cybernetic living, user 100 can be provided with a holistic view of all of their digital assets, and a plan that can be automatically executed in the event of an incident. The system can take best-in-class approaches from enterprise security programs, and implement them for individuals.

Digital information is a core part of everyone's life. The described system allows users 100 to always know how their personal and business information is being protected. The system can perform a security assessment of user's 100 digital life, identify any deficiencies, create a remediation plan, and fix anything that may be broken. All of the data regarding user's 100 security outcomes may be integrated into user's 100 personal data model and personal operating system, so that it is a cohesive part of user's 100 daily routines and digital life.

### Instrument Life's Critical Processes

In at least one embodiment, the system can provide an instrumented flow of the critical processes associated with user's 100 life. The system can automatically discover and review the critical processes that make up user's 100 life, and turn them into step-by-step runbooks. Engineers can build personalized runbooks for user 100 with an implementation plan to support in experimenting with various solutions contrived by user's 100 data. This enables user 100 and others on their team to execute tasks easily and flawlessly because they know where all the assets are to get the job done. User's 100 personal digital runbooks can be made available digitally to anyone they designate on their personal board of directors.

### Timeline 300

In at least one embodiment, the system includes a timeline viewer interface 301 that provides a timeline 300, which may be an integrated, interactive view of user's 100 past, present, and future.

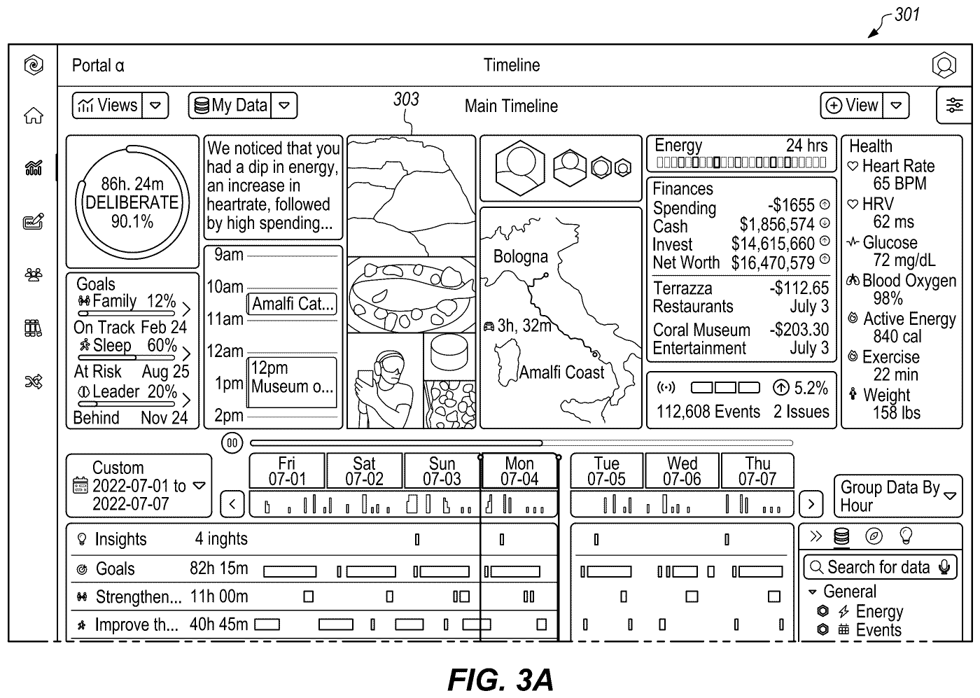

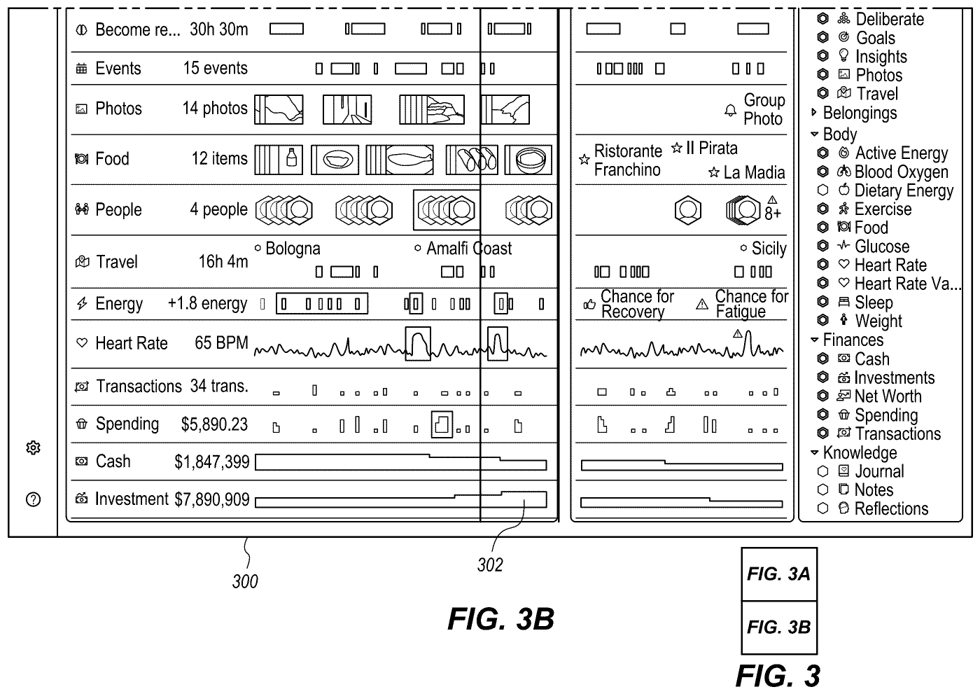

Referring now to FIG. 3, there is shown an example of a timeline viewer interface 301 for presenting unified timeline view 300 (or “timeline”). Timeline viewer interface 301 may be presented on an output device such as display screen 103 of device 101 or 108. Playhead 302 may be displayed, and may move horizontally across timeline 300 as detailed data for a point in time corresponding to playhead 302 is displayed. In at least one embodiment, summary panels 303 along with other displayed data correspond to the currently active timeframe as indicated by playhead 302.

As shown in the example of FIG. 3, timeline 300 may include visualizations of a time-centric view of user's 100 data. Some of this data may be direct transformations of samples of quantitative data (e.g. heart rate, weight) to time-series values, retrieved from data store 106 and/or other storage components. Other processes may provide data for timeline 300 using models or projections to normalize, convert, and transform data that may be irregular, may contain gaps, or may be qualitative data interpreted with user-specific values into time-series metrics.

In at least one embodiment, timeline 300 allows user 100 see their data in a centralized manner so that they can conduct synchronous correlation discovery and easily track progress, trends, and patterns across time.

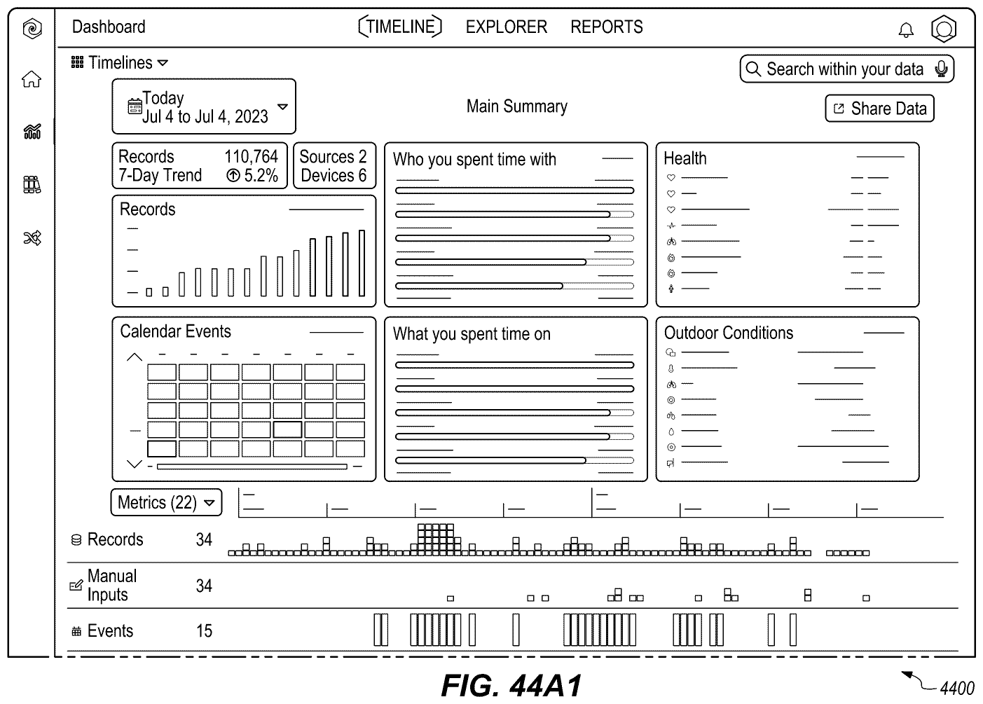

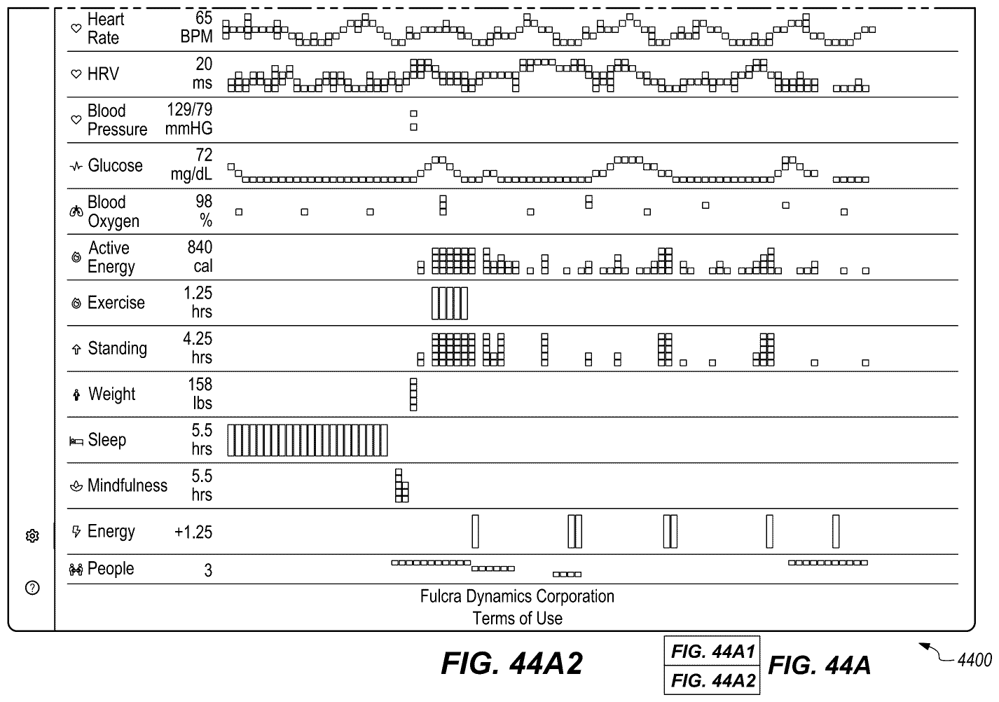

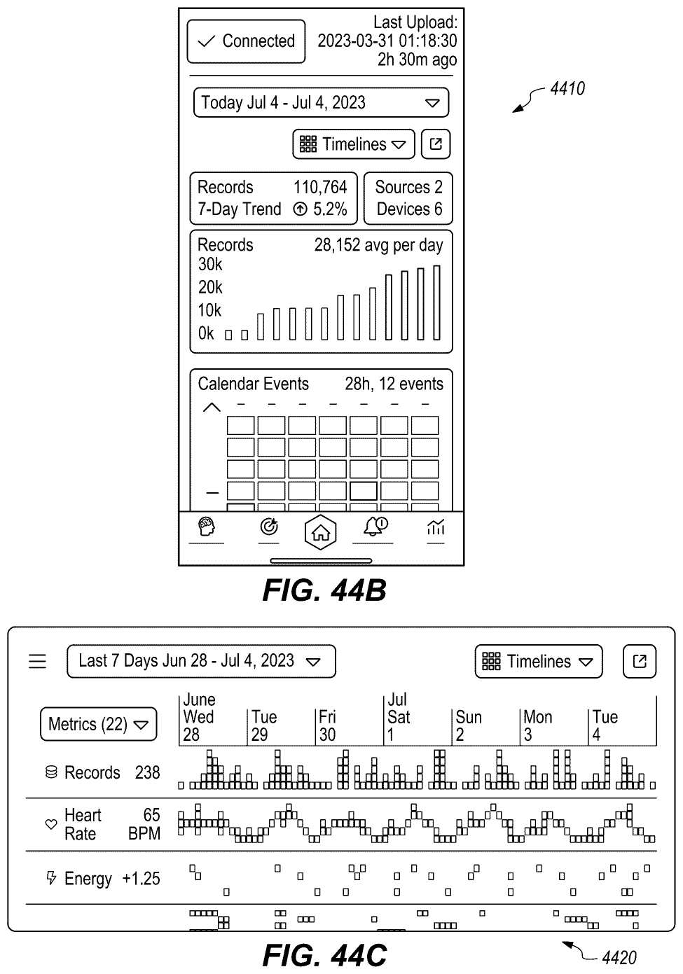

Referring now to FIG. 44A, there is shown a diagram depicting a main summary display 4400 for timeline 300, according to one embodiment.

Referring now to FIG. 44B, there is shown a diagram depicting a mobile display 4410 including visualizations. Referring now to FIG. 44C, there is shown a diagram depicting a mobile display 4420 including metric tracks. In at least one embodiment, display 4410 may be shown when the device is held in portrait mode, and display 4420 may be shown when the device is held in landscape mode. Display screen 103 of device 101 may switch automatically between display 4410 and 4420 when the orientation of device 101 is changed.

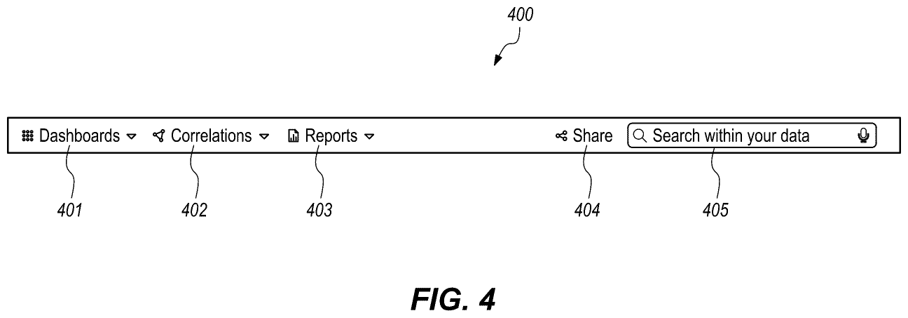

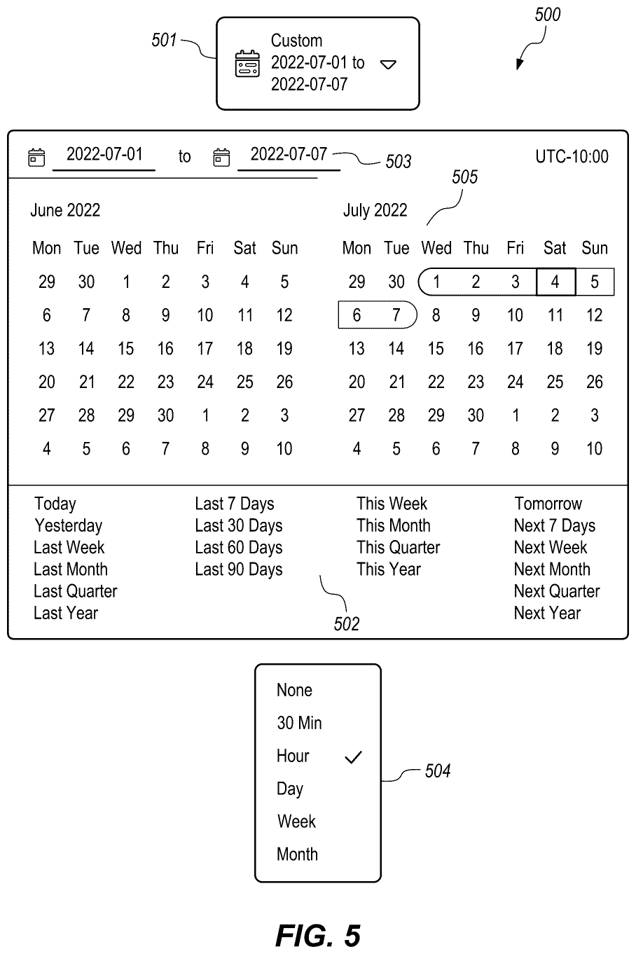

In at least one embodiment, the system provides navigation tools to allow user 100 to control the selected date range, select another timeline 300, share the selected timeline 300, and/or otherwise configure the display of timeline 300. Additional details regarding such navigation are provided herein in connection with FIGS. 4 and 5.

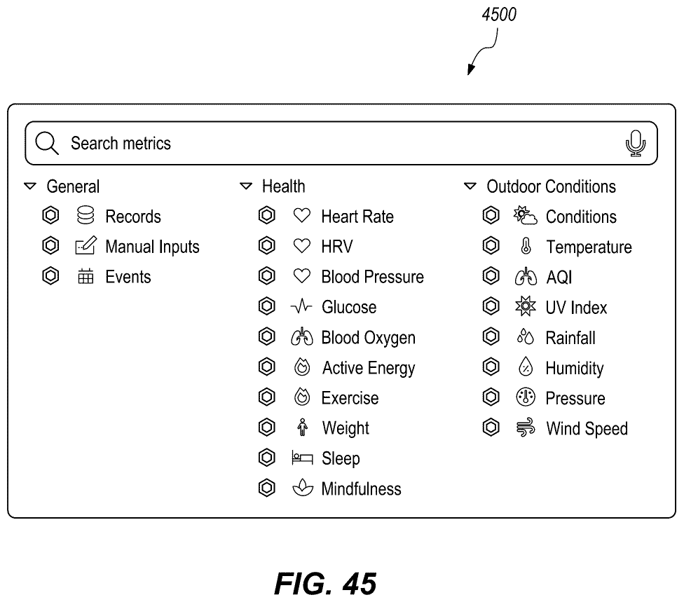

Referring now to FIG. 45, there is shown a diagram depicting screen 4500 for selecting and/or deselecting metric tracks that are visible on timeline 300, according to one embodiment. By default, in at least one embodiment, all available metrics are selected. User 100 can configure timeline 300 by selecting or deselecting metrics according to their wishes.

### Method

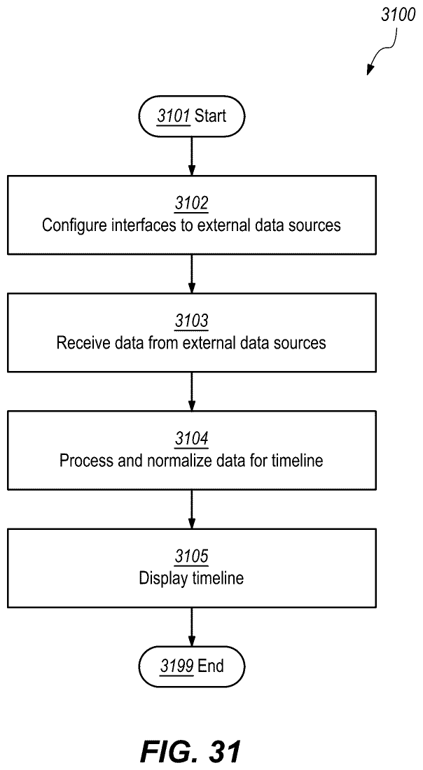

Referring now to FIG. 31, there is shown a flow diagram depicting a method for generating and displaying timeline 300, according to one embodiment.

The method begins 3101. In step 3102, the system configures interfaces to external data sources. These interfaces may be static or dynamic; a static interface allows collection of a snapshot of data relating to user 100 at a given point in time, while a dynamic interface allows collection and continuous (or continual) update of data relating to user 100. In at least one embodiment, such interfaces may be configured using datashares, as described in more detail herein. The external data sources may be of any suitable type, including for example, wearable devices, computing devices, cloud-based sources, smartphones, tables, and/or the like. Interfaces to such devices may operate over any known communication network, whether wireless or wired, as appropriate, and may use any known protocols for such electronic communications.

Once interfaces have been configured, the system may, in step 3103, begin to receive data from the external data sources. This step may be performed once, or multiple times, or on a continuous or continual basis. The data received from the external data sources may be of any type that may be useful in generating and displaying unified timeline view 300. Examples of the type of data received may include: health-related data for user 100, data describing user's 100 browsing habits, purchase history, social media habits, activity, travel, location, and/or the like.

In step 3104, the received data may then be processed and/or normalized for display in a unified manner in timeline 300. In step 3105, timeline 300 may then be generated and displayed. In at least one embodiment, timeline 300 may be interactive, allowing user 100 to scroll, enlarge, edit, and/or otherwise configure timeline to their liking.

The method then ends 3199.

### Purpose and Use Cases

In at least one embodiment, timeline 300 is a tool that allows user 100 to explore their past, present, and future in a time-based manner with greater fidelity and certainty than are permitted by existing techniques. User 100 is able to see a rich media replay of their life so they can understand hidden trends and correlations across different areas of their life. They are then able to see a forecast of what is in store for them so they can plan and make decisions accordingly. Having the ability to look back and forward helps users 100 to better understand who they are, measure how they are improving, and further train their intuition to make reflection and decision making an easier process over time.

### Design Rationale

In at least one embodiment, timeline viewer interface 301 can support increasing automation and future looking recommendations. As the system is able to integrate more data sources, it can populate future-looking elements of timeline 300 with known plans and events. In at least one embodiment, the system can include an engine that can analyze past events for patterns and make predictions for the future, so as to offer better decision support to users 100 with recommendations for actions they can take for the best outcomes. In at least one embodiment, the system can provide user 100 with the ability to configure their own dashboards and visualizations to go beyond what FDE 251 sets up for them to see.

### Navigation

Referring now to FIG. 4, there is shown an example of a navigation section 400 of timeline viewer interface 301, according to one embodiment.

Dashboard menu 401 provides access to functionality to allow user 100 to save a Dashboard configuration for easy access later.

Correlations 402 menu allows user 100 to access links to the datasets with interesting and unexpected correlations that have been surfaced to them by the system or by FDE 251.

Reports menu 403 provides access to functionality to allow user 100 to create ad hoc reports of specific data from a particular date range or create recurring reports with a set data configuration.

Share menu 404 provides access to functionality to allow user 100 to create an ad hoc snapshot of the data currently selected on timeline 300 to share with other people. Sharing options may include, for example:
Format: Image [PNG], video [MOV], and/or the like;
Method: Download, text, social media, email, and/or the like.

Search Within Your Data field 405 allows user 100 to search for any desired keywords within their own data.

### Date and Time Granularity Selections

Referring now to FIG. 5, there is shown an example of a section 500 of timeline 300 for selecting date and time granularity, according to one embodiment.

User interface controls 501, 502, 503, and 505 allow user 100 to easily select a date range so that they can visualize data from certain points in time. User 100 can also easily specify the granularity of the data via menu 504, which controls how the data is aggregated by time so that they can visualize their data in a way that makes it easy to see patterns or view multimedia playback.

Manual input section 503 may be provided to allow user 100 to manually input a date range into start date and end date textboxes.

If the date range matches a quick pick date range from quick pick list 502, then it may be shown as selected, and the date range label may be displayed in the dropdown menu. In at least one embodiment, the format does not need to match exactly for the date to be valid; for example, dates with matching YYYYMMDD may be automatically formatted to YYYY-MM-DD upon selection of textbox.

Alternatively, user 100 can select date ranges from quick pick list 502 including a number of preset date ranges. In at least one embodiment, on selection of a date range from the quick pick list 502, the dates within textboxes 503 and calendar 505 automatically update. The date range label may be shown in a dropdown menu. The lists may be separated by past dates and future dates to make it easier to find a date range.

In at least one embodiment, user 100 can select a date range from displayed calendar 505. The dates within textboxes 503 may automatically update, and the selected dates may be highlighted in calendar 505. In at least one embodiment, for example, past days may be highlighted in “Light Purple—75%”, today's date may be highlighted in “Light Purple” with bold “Contrast” text, and future days may be highlighted in “Light Purple—25%”. In at least one embodiment, if the date range matches a quick pick date range in list 502, then it may be shown as selected and the date range label may be displayed in the dropdown menu.

In at least one embodiment, user 100 may select a level of time granularity from menu 504. This may specify a manner in which the data will be displayed, and may also specify a level of granularity of the data as well. Options may include, for example:
None
30 Min
Hour
Day
Week
Month

In at least one embodiment, user 100 can also specify a time zone.

### Visualization Panel

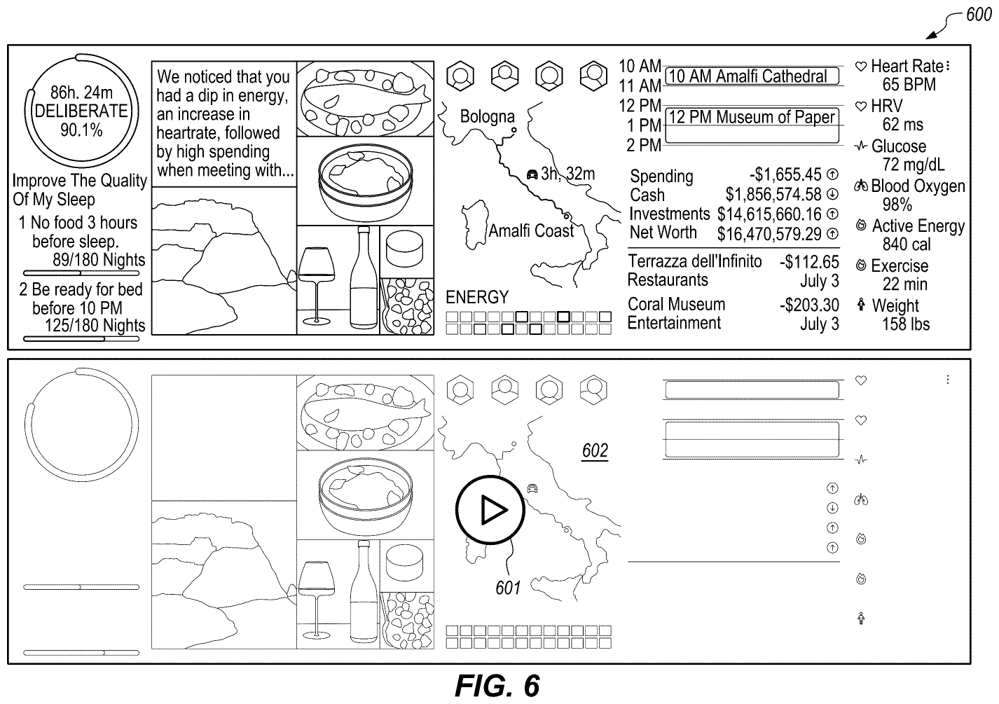

Referring now to FIG. 6, there is shown a display 600 in which user's 100 data may be played in a visual format, according to one embodiment. Play button 6oi may be presented when user 100 causes the cursor to hover over summary panel 602, allowing user 100 to quickly play back their data. As described in more detail below, playhead 302 and a progress bar may be provided to provide user 100 with more control over playback.

### Side Panel

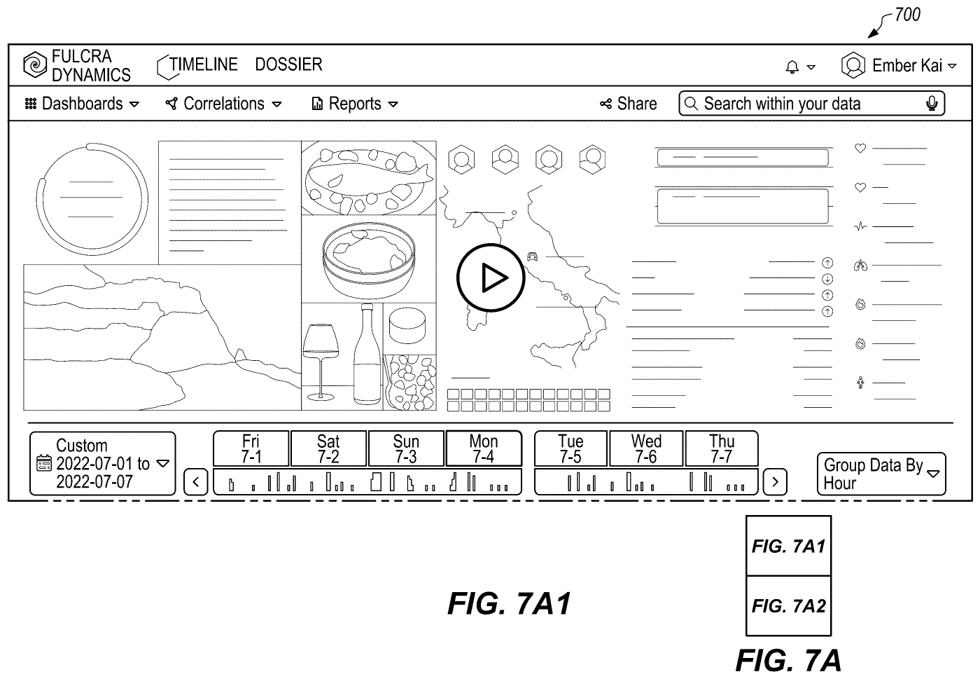

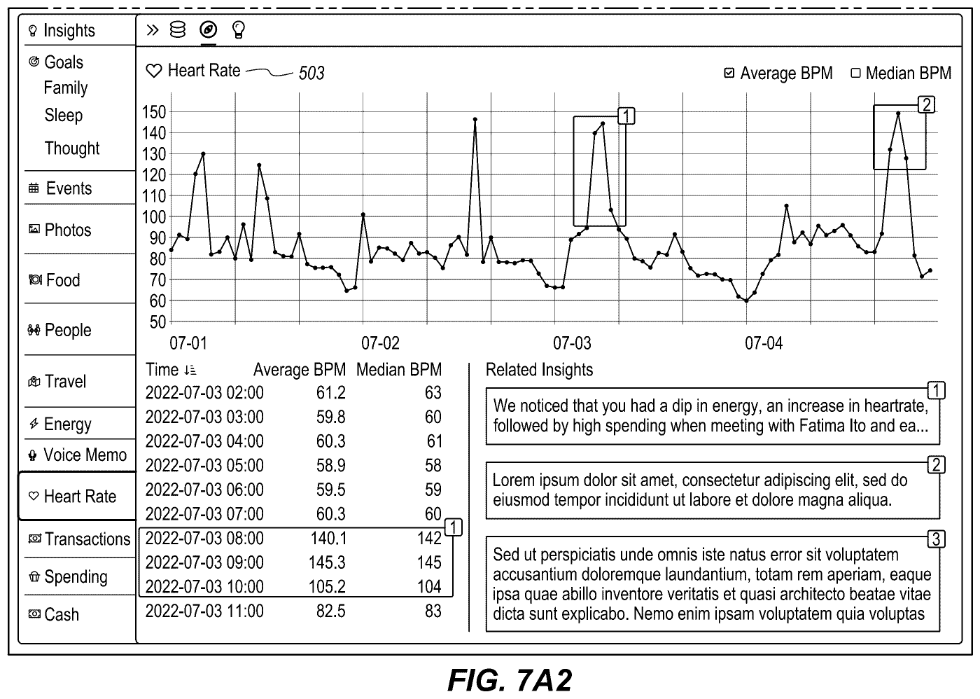

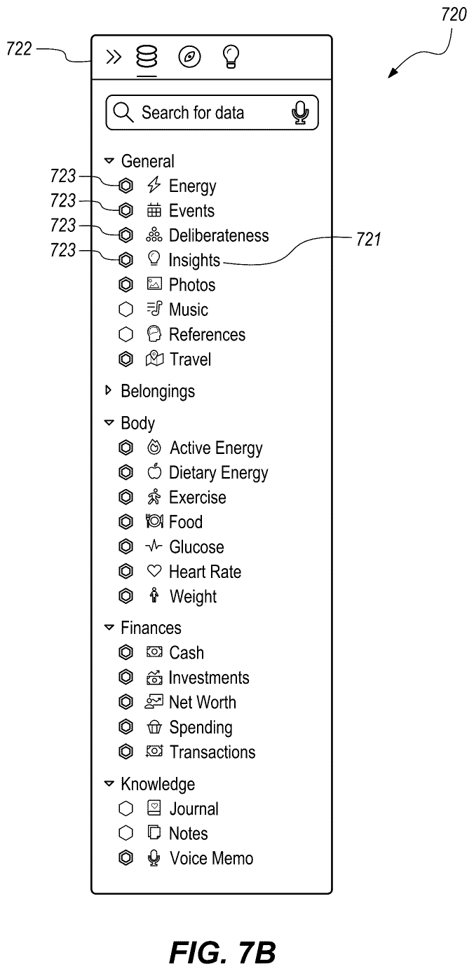

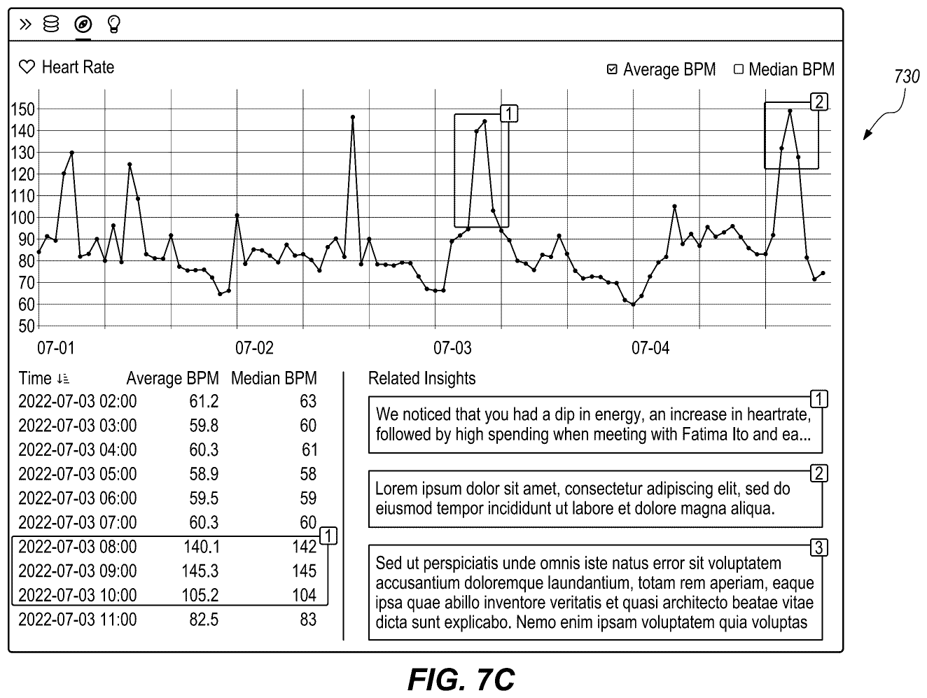

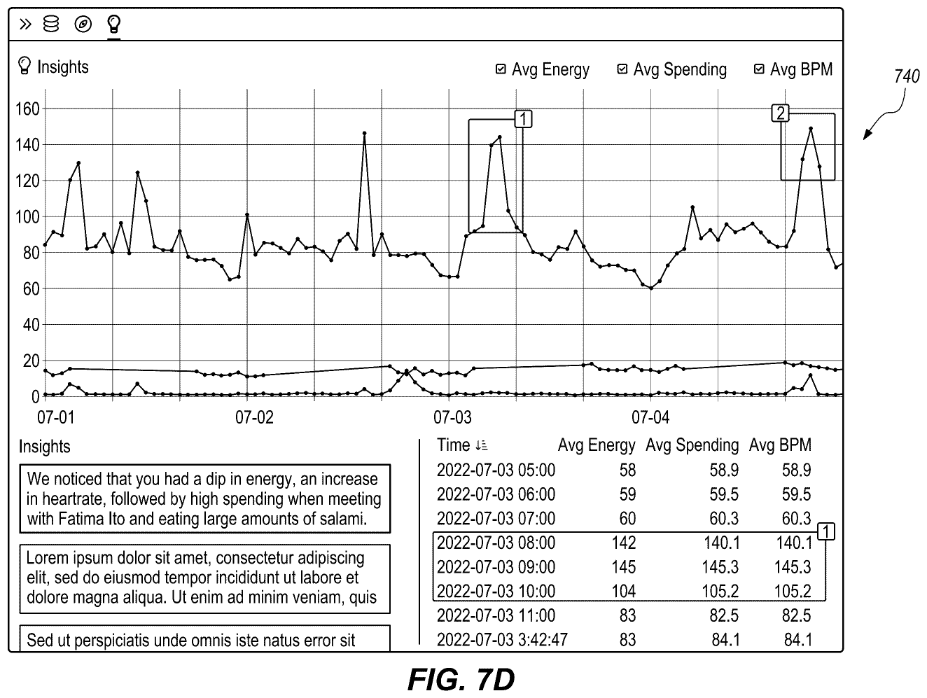

Referring now to FIGS. 7A through 7D, there is shown an example of the operation of a Side Panel 720 that allows user 100 to quickly view more detailed information, thus providing a way for user 100 to explore their data, while maintaining the context of the starting point where they see something in the data shown in the main (default) timeline view 301 that they would like to investigate in more detail.

Side Panel 720 provides user 100 with access to more options and detailed information while viewing the Timeline. As shown in screen 700 of FIG. 7A, in at least one embodiment, the default state of the Side Panel 720 (not visible in FIG. 7A) is collapsed to the right with the DataStream tab selected. User 100 may click on a DataStream label 503 within the table (such as, for example, Heart Rate). As shown in FIG. 7B, Side Panel 720 then extends to show the Explorer with detailed data for that particular DataStream.

User 100 may click Insights tab 721 within Side Panel 720 to cause a list of all insights for the selected time period to be displayed.

User 100 may click Collapse button 722 to cause Side Panel 720 to slide all the way to the right, displaying only the icons vertically in a bar.

FIG. 7B depicts an example of a DataStream tab that allows user 100 to select the data they would like to visualize. By clicking on checkboxes 723, user 100 can select the DataStreams feeding the Timeline to customize their dashboards.

In at least one embodiment, user 100 can use this tab to select or deselect the DataStreams to be displayed on the dashboard. Clicking on a radio button or label causes the corresponding DataStream to be presented in a visual format in the visualization panel on top as well as the data table on the bottom.

In at least one embodiment, default selections may include:
Deliberate
Energy
Events
Goals
Insights
People
Photos

In at least one embodiment, a default list order may include categories listed in alphabetical order, with the exception of “General” which may be listed as the first category. The DataStream may be listed in alphabetical order within categories.

In at least one embodiment, a limit may be placed on the number of DataStreams that may be concurrently selected, such as example a limit of 20.

In at least one embodiment, functionality may be included to allow user 100 to search for DataStreams.

In at least one embodiment, clicking a header causes the list of DataStreams within the category to collapse and the icon to toggle.

In at least one embodiment, user 100 can explore a particular DataStream in more detail by selecting it. This causes the underlying data table to be displayed, allowing user 100 to see any related insights that may be annotated for them by the FDE.

Exploring a specific DataStream allows user 100 to take a closer look at the patterns and anomalies in the data over time while being able to jump to annotated portions of the data related to Insights.

In at least one embodiment, color coding and/or labeling may be used. For example, the metric label and colors may match the chart, legend, and table headers. Highlights for the Insight may be displayed on the chart with number labels that match the highlights on the data table and Related Insights table. User 100 may click a metric in the legend to cause the data visibility in the chart to be toggled. Alternatively, at least one embodiment, hiding a metric on the chart does not toggle the visibility of the metric in the data table.

In at least one embodiment, the chart may display all data related to the selected DataStream. User 100 can click a highlight on the chart to cause the data table to scroll to the highlighted part of the data and Related Insight.

In at least one embodiment, the data table may present data in a default sort order from oldest to newest. Clicking on the header of a data column may toggle the sort, for example, between descending, ascending, and/or none/default.

In at least one embodiment, Related Insights may be sorted by the sort selected on the data table to the left. Related Insights may be labeled to match the labels on the data table and chart.

In at least one embodiment, user 100 may select View All Insights to see all Insights created for them in one place for a specific time period, allowing user 100 to look at the related data in more detail and see how it is correlated. In this manner, by displaying the chart and underlying data table with the Insight, the system makes it easy to demonstrate the correlations described in the Insight between the selected metrics at certain points in time. The Insights tab may display all insights created for user 100 within the selected date range.

Referring now to FIGS. 7C and 7D, there are shown additional examples of screens 730, 740 for displaying data associated with user 100.

Screen 730, shown in FIG. 7C, is an example of how a user can select an individual DataStream so that they can explore the data in more detail with the underlying data table and see any related insights annotated. Exploring a specific DataStream allows user 100 to take a closer look at the patterns and anomalies in the data over time while being able to jump to annotated portions of the data related to Insights.

Screen 740, shown in FIG. 7D, is an example of how a user can see all of the Insights created for them in one place for a specific time period so that they can look at the related data in more detail and see how they are correlated. The main goal of displaying the chart and underlying data table with the Insight is to make it easy to demonstrate the correlations described in the Insight between the selected metrics at certain points in time. The Insights tab will display all insights created for user 100 within the selected date range. In at least one embodiment, these insights may be generated by an LLM.

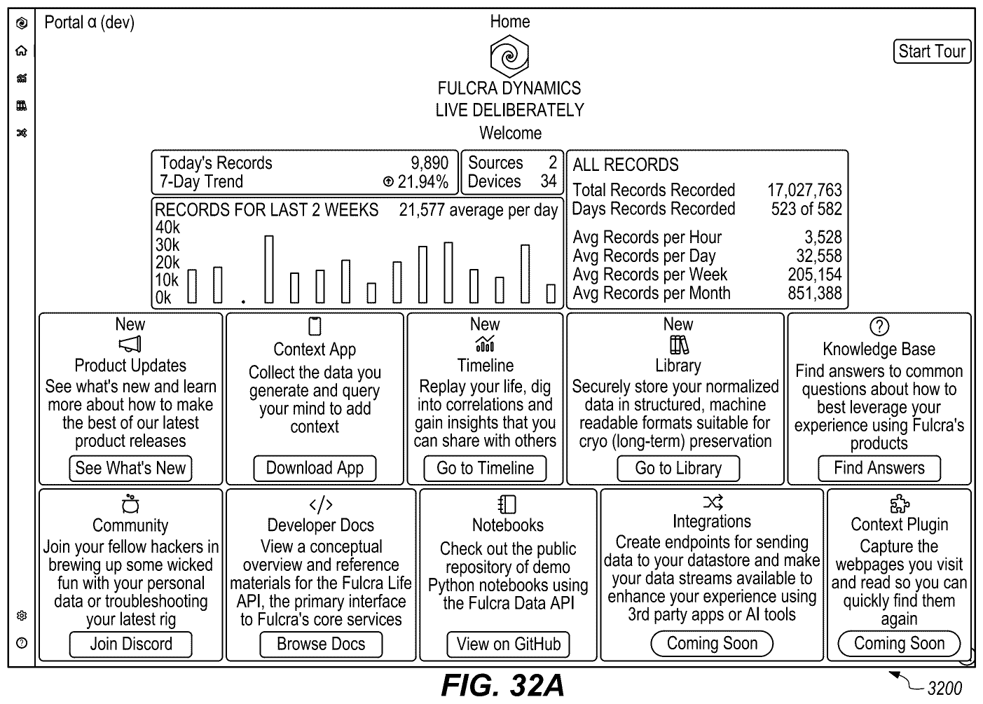

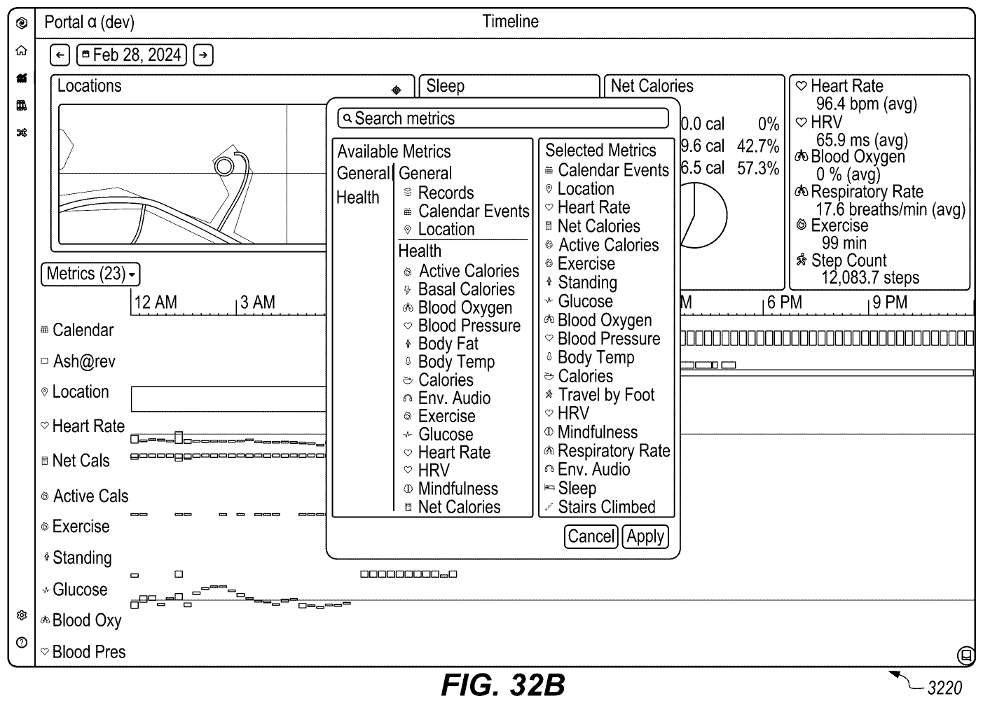

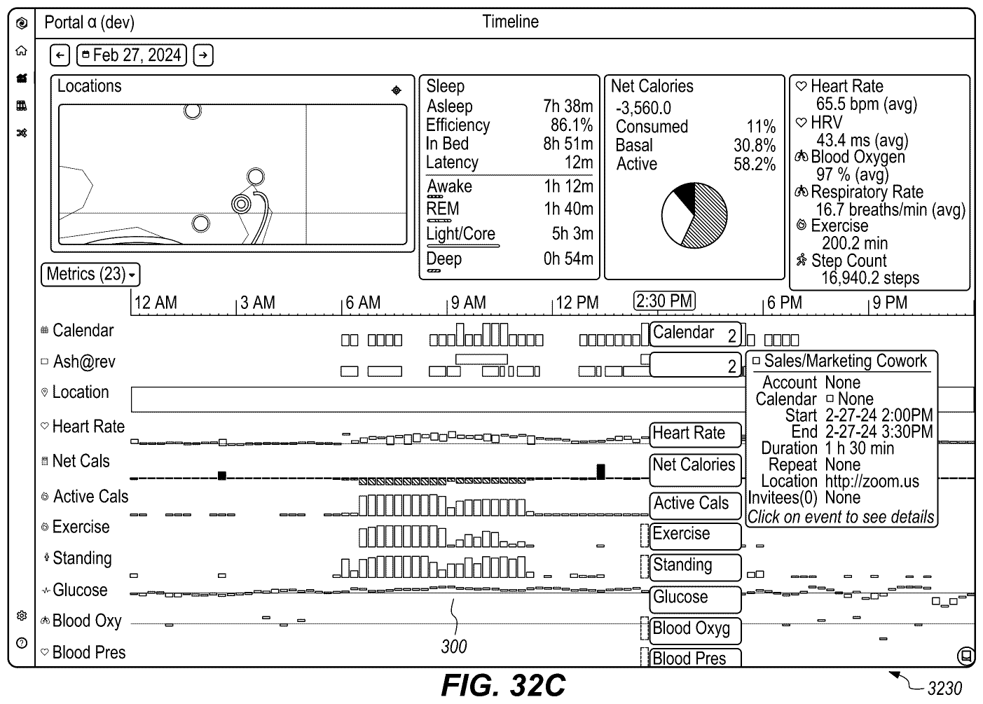

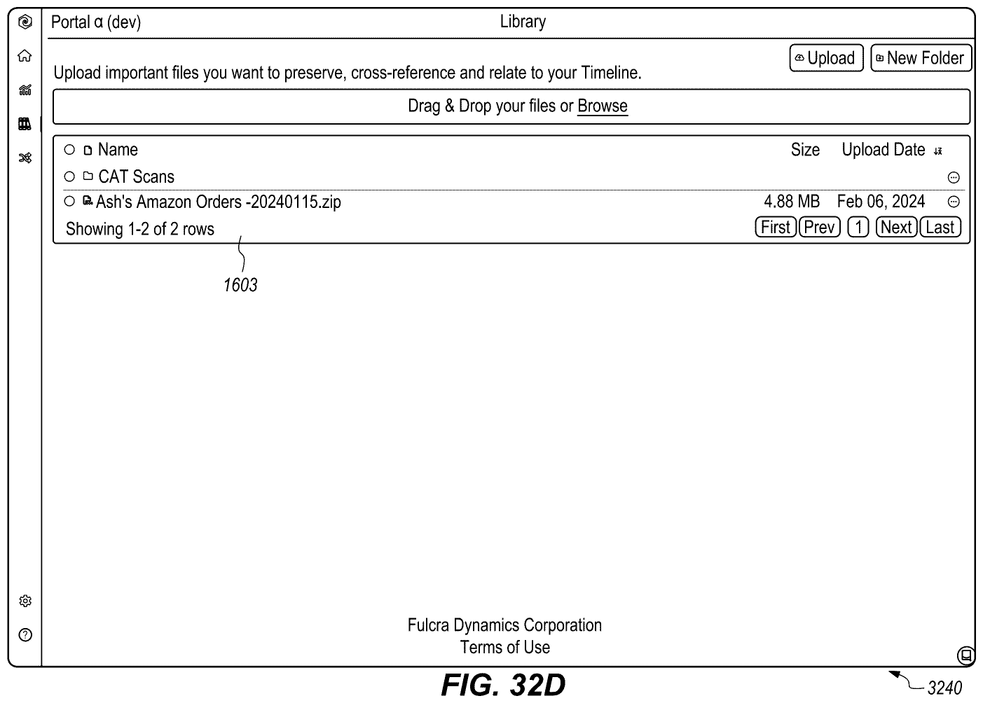

Referring now to FIGS. 32A through 32D, there are shown additional examples of screens 3200, 3220, 3230, 3240 for display of timeline 300 and related elements.

FIG. 32A depicts an example of Portal Home page 3200, including a quick summary of user's 100 records so that they can easily check if there are any issues with data collection.

FIG. 32B depicts an example of metric selection menu 3220 where user 100 can view metrics by category and search for individual metrics.

FIG. 32C depicts an example of screen 3230 for displaying current timeline 300. User 100 can hover over any part of the data rows of timeline 300, causing a tooltip to appear displaying a summary of all the rows in the first column and the details of the particular row being hovered over for a particular time period, such as a 15-minute chunk of time.

FIG. 32D depicts an example of screen 3230 for displaying library 1603 where user 100 can upload files to save to their personal datastore. These files may then be parsed and normalized by the data service so that it can be accessed by user 100 from the Life API and configured to be displayed on timeline 300.

### Playback

In at least one embodiment, a playback function may be provided. Upon activation of the playback function, the system presents a dynamic display showing data at successive times along timeline 300. As described above in connection with FIG. 3, playhead 302 may be displayed, and may move horizontally across timeline 300 as detailed data for a point in time corresponding to the playhead is displayed. In at least one embodiment, user 100 can specify the start and end times for the playback, and can specify the speed at which playback occurs. In at least one embodiment, a pause button may be provided to allow user 100 to pause playback. Additional controls may be provided for going forward or backward by a predefined amount of time (e.g. 1 hour, or 15 seconds, or any other time period), or resuming previously paused playback. In at least one embodiment, the playhead may be an interactive control, which user 100 may slide horizontally to various points along timeline 300. Displayed data may be dynamically updated to reflect the current position of playhead as it moves or is moved by user 100. In at least one embodiment, as shown in FIG. 3, summary panels 303 along with other displayed data correspond to the currently active timeframe as indicated by playhead 302.

### Calendar Events

In at least one embodiment, the system provides functionality to allow user 100 to see events from their calendar, displayed on timeline 300. This allows user 100 to see what they were doing at particular times and compare to other metrics on timeline 300.

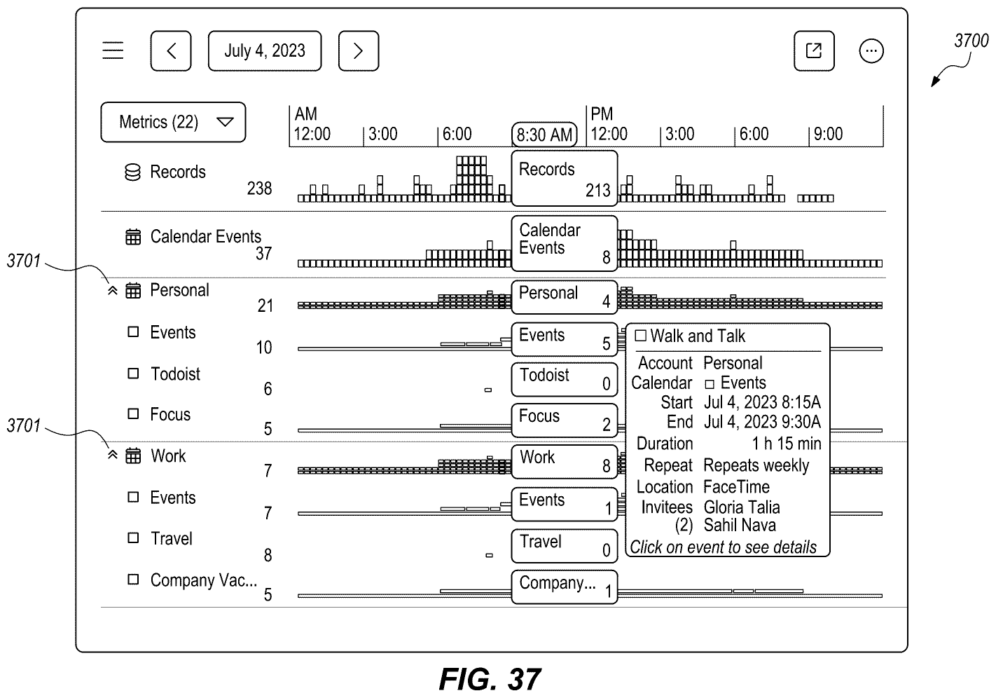

Referring now to FIG. 37, there is shown an example of a Calendar Events screen 3700.

In at least one embodiment, there is a hierarchy of metric rows to represent calendar events, as follows:

| Type | Display | Chart |
|---|---|---|
| Calendar | Total number of events across all | Rollup bar |
| events | accounts displayed as a sum for each | |
| | time increment | |
| Account | Total number of events across | Rollup bar |
| | calendars for an individual account | |
| | displayed as a sum for each time | |
| | increment (e.g., Personal, Work) | |
| Calendar | Individual events for an individual | Gantt |
| | calendar displayed as horizontal spans | |
| | of time according to their start and end | |
| | times (e.g., Walk and Talk) | |

In at least one embodiment, user 100 can click on chevron icons 3701 to expand or collapse calendar rows contained within the account. The accounts may initially be collapsed by default.

In at least one embodiment, calendars may be sorted alphabetically first by account name and then by calendar name in the metrics list by default. In alternative embodiments, other sorting mechanisms can be user.

In at least one embodiment, individual events may be displayed using a Gantt chart as horizontal spans of time. The bars may be solid for the duration, and a small space may be provided between consecutive events so as to distinguish between the start and end of events.

In at least one embodiment, overlapping events may be displayed on new rows until the horizontal space runs out. All-day events may span the entire width of the day. The height of the calendar row for events may be divided until there are up to three rows before adding height to the calendar row to include new event rows.

In at least one embodiment, hovering over an individual calendar event causes a tooltip bar with a summary column with the total number of events within a row and an event detail column to be displayed. The color of the calendar label may match the calendar color that user 100 assigned on their Calendar application. The tooltip may be moved horizontally to adjust to display the full width of the summary plus event detail view. The vertical position of the detail view may align with the top of the calendar the event belongs to and may swing to the bottom or top of the event's vertical position to adjust, depending on the vertical space available.

In at least one embodiment, user 100 can click on the event bar to see a modal dialog box with full event details.

### Location

In at least one embodiment, the system provides functionality to display user's 100 location within timeline 300.

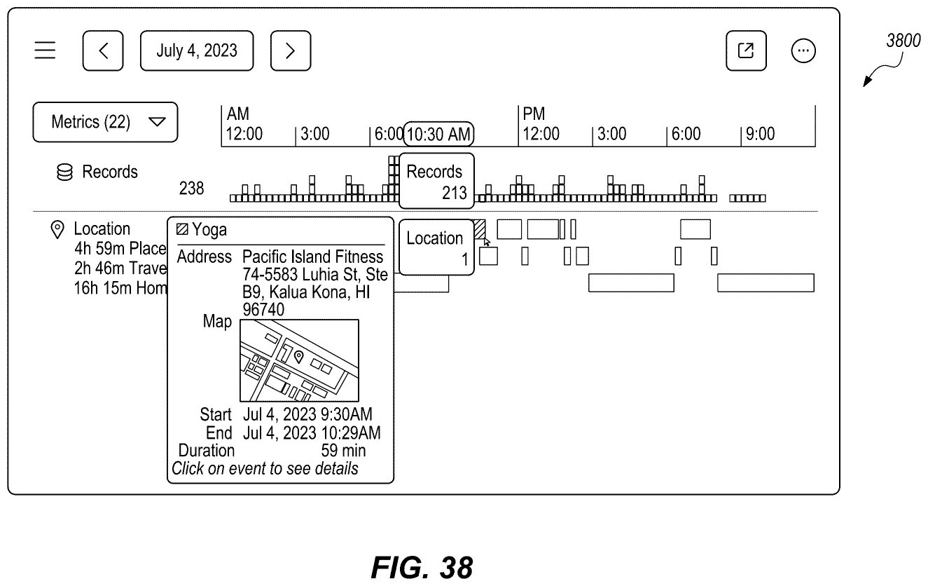

Referring now to FIG. 38, there is shown an example of a Location screen 3800.

In at least one embodiment, three distinct rows may be provided to help user 100 distinguish between different default type of location data, which may describe: places they visited, time of visit, and time at home.

In at least one embodiment, summary metrics may be provided to indicate total durations that user 100 was at certain places (Places), traveling (Travel), or at home (Home).

In at least one embodiment, user 100 can hover over a bar to see a tooltip including a summary metric, which may include a count of places (including home) that user 100 visited, and whether user 100 traveled in the corresponding time span. A second tooltip may provide additional details about the place, travel, or home.

### Metrics

In at least one embodiment, the system provides functionality to customize metrics within timeline 300.

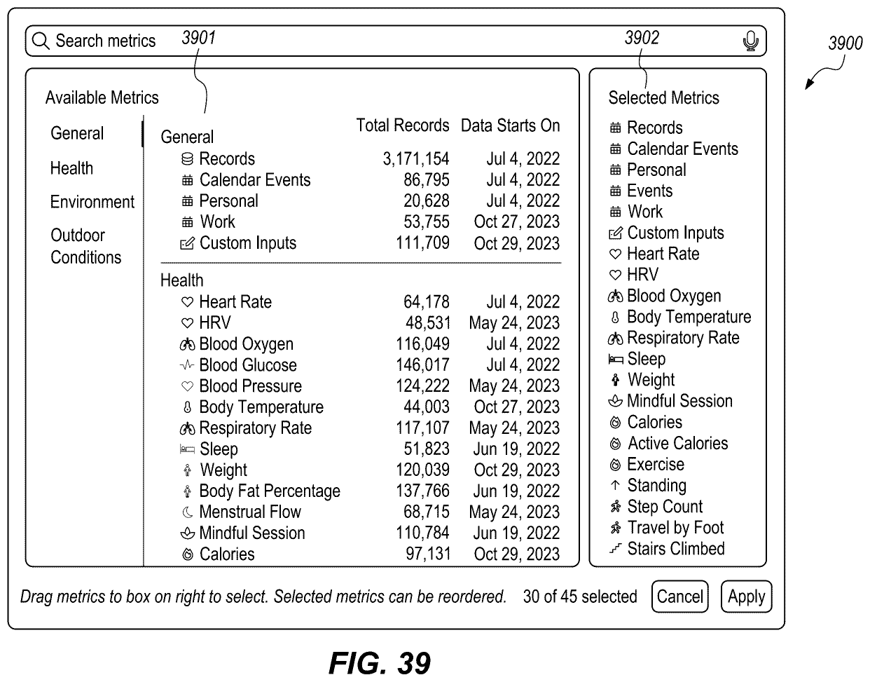

Referring now to FIG. 39, there is shown an example of a Metrics screen 3900.

In at least one embodiment, user 100 can select metrics to appear on the Timeline by dragging one or more metric(s) from Available Metrics list 3901 to Selected Metrics list 3902. The icon of the selected metric(s) turns purple to indicate that it/they are selected. Mobile layouts can stick with using the checkbox to indicate selected metrics. To deselect a metric in Available Metrics list 3901, user 100 can hover over a metric and a red X appears at the right-hand edge of the row. On click of the red X, the metric may be removed from the list.

In at least one embodiment, user 100 can reorder metrics by dragging the metric row up or down within Selected Metrics list 3902. If user 100 hovers over a metric row, a grab (hand) cursor may appear. If user 100 drags the cursor, a horizontal purple bar may appear between the rows to help user 100 target the new placement of the row.

### Metric Tracks

In at least one embodiment, the system provides various metric tracks for displaying data within timeline 300. These metric tracks may be presented in a uniform manner to enable comparison of trends across tracks while saving vertical space.

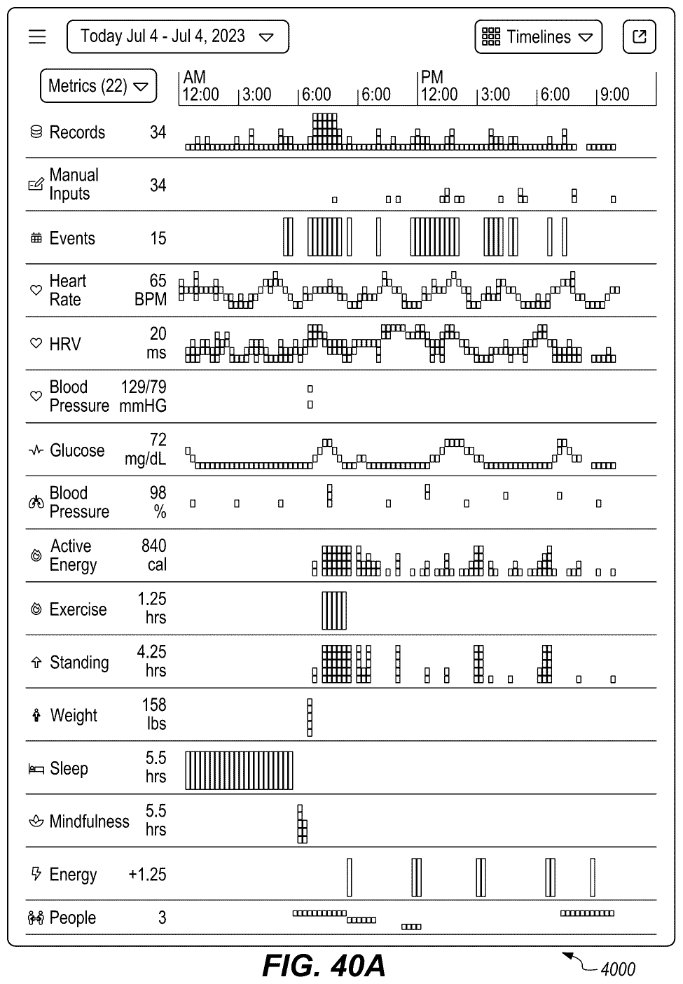

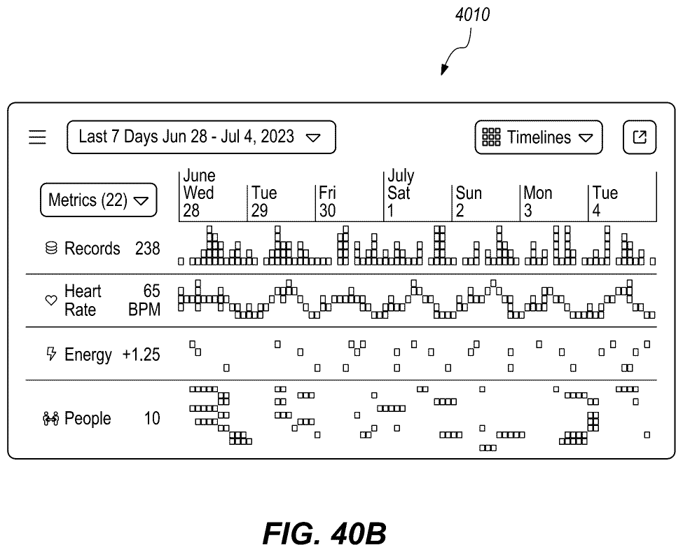

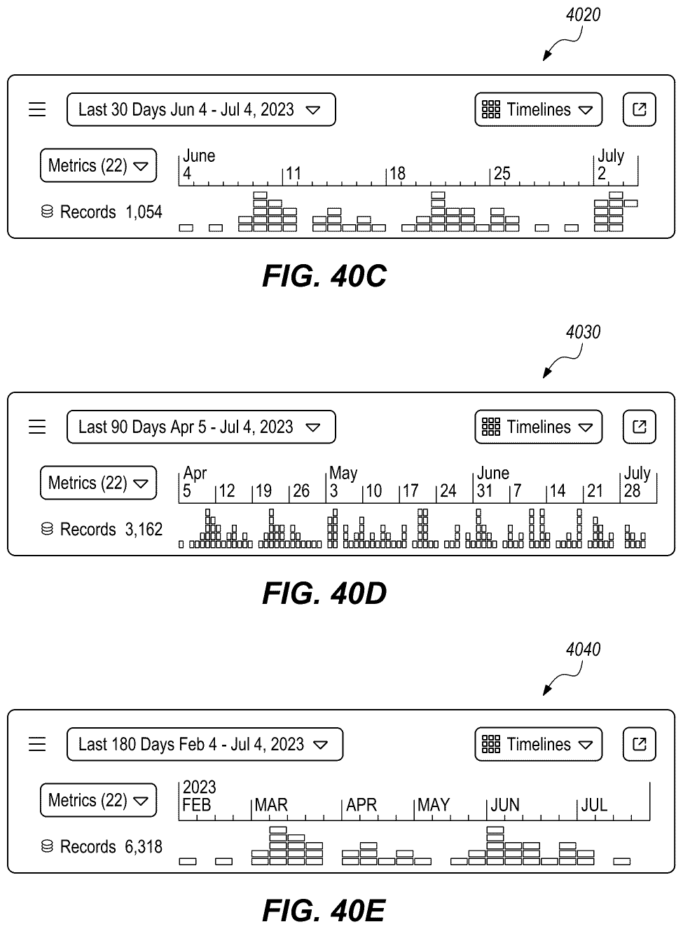

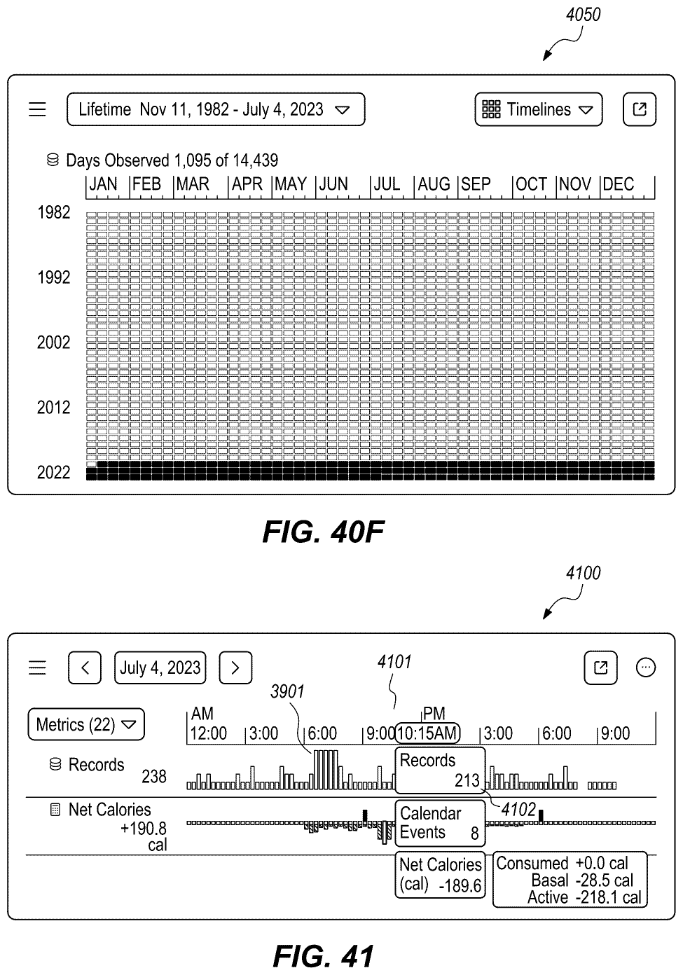

Referring now to FIG. 40A, there is shown an example of a display 4000 including various metrics presented across a one-day period of time.

Referring now to FIG. 40B, there is shown an example of a display 4010 including various metrics presented across a one-week period of time.

Referring now to FIG. 40C, there is shown an example of a display 4020 including various metrics presented across a 30-day period of time.

Referring now to FIG. 40D, there is shown an example of a display 4030 including various metrics presented across a 90-day period of time.

Referring now to FIG. 40E, there is shown an example of a display 4040 including various metrics presented across a 180-day period of time.

Referring now to FIG. 40F, there is shown an example of a display 4050 including various metrics presented across the entire period of time for which data is available for user 100.

### Net Calories

In at least one embodiment, the system provides functionality to display user's 100 net calories (intake vs. burned off) within timeline 300.

Referring now to FIG. 41, there is shown an example of a display 4100 of net calories. Net calories may be presented in terms of calorie intake, less basal (expended by the body at rest), less active (expended through physical activity).

In at least one embodiment, hovering over a bar activates tooltip 4101 displaying a summary metric, including net calories for the corresponding time span. Breakdown of intake (consumed), basal, and active calories may be displayed in a second column 4102 of tooltip 4101.

### Sleep

In at least one embodiment, the system provides functionality to display user's 100 sleep data within timeline 300.

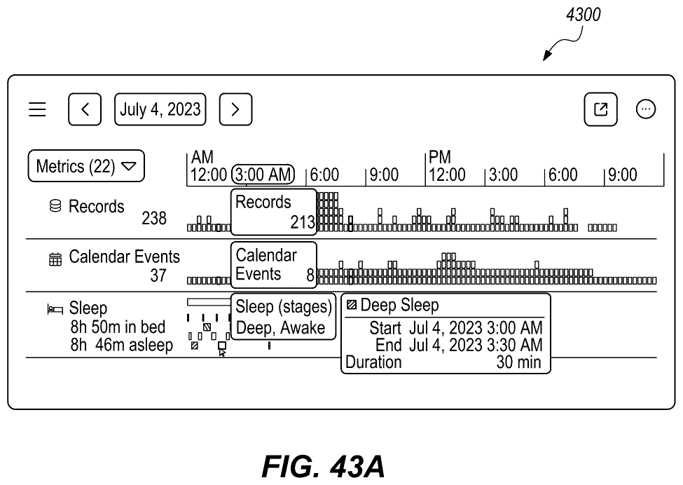

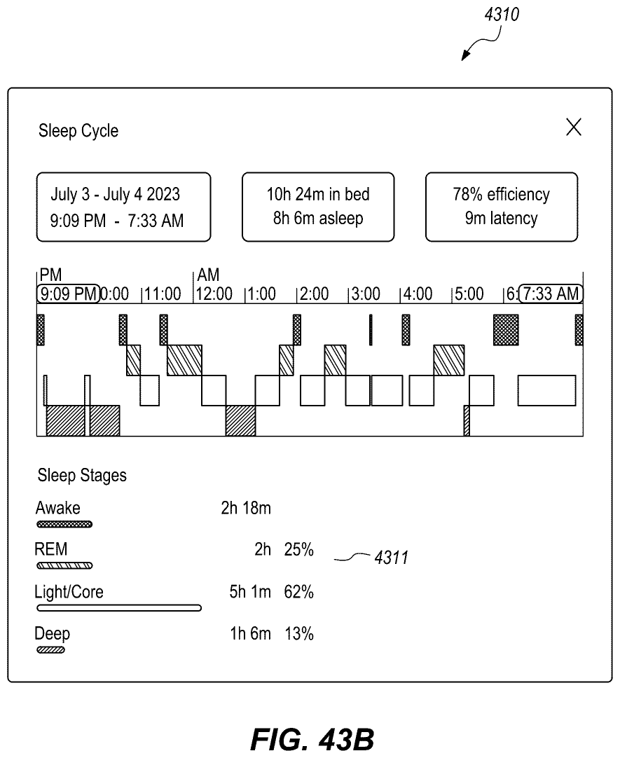

Referring now to FIGS. 43A and 43B, there are shown examples of display 4300, 4310 of sleep data. In at least one embodiment, two cumulative metrics can be displayed: “in bed” and “asleep.” Various sleep stages may be tracked, including for example In Bed (inferred) Awake, REM, Light/Core, and Deep. Each may be displayed on a distinct row within display 4310, with coloring, as follows (examples only):

| Stage (Row) | Description | Color |
|---|---|---|
| In Bed | User 100 is in bed | Primary green |
| (Inferred) | | |
| Awake | User 100 is awake | Yellow |
| REM | User 100 is in REM sleep | Light green |
| Light/Core | User 100 is in light or intermediate | Blue |
| | sleep | |
| Deep | User 100 is in deep sleep | Dark purple |

In at east one embodiment, a sleep efficiency score may also be calculated.

In at least one embodiment, the In Bed (inferred) stage may be calculated based on the time user 100 is in bed or asleep.

In at least one embodiment, if user 100 hovers over a segment, a tooltip may be displayed, showing the particular sleep stage(s) that occurred during the segment. Details may be displayed in a larger tooltip in a second column.

In at least one embodiment, in response to clicking anywhere within the metric row that is within the In Bed time span, display 4310 may be shown, including the entire sleep cycle for the night. Sleep stages 4311 may show cumulative/categorical metrics for each sleep stage.

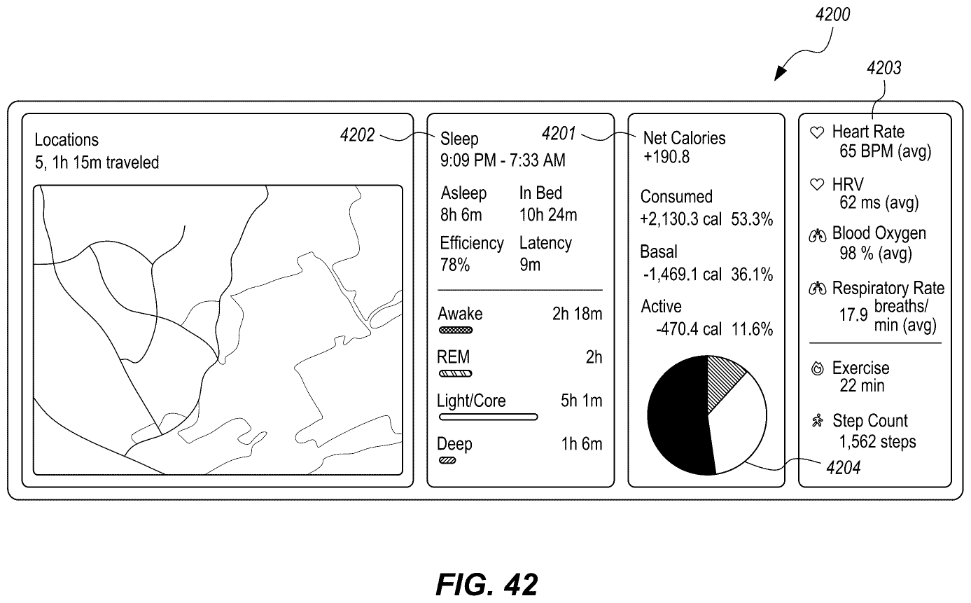

In at least one embodiment, user 100 may cause the system to play back sleep history, which causes cumulative sleep data from the beginning of the sleep cycle to be displayed as a playhead moves across timeline 300. In response to user 100 hovering over timeline 300, cumulative sleep values for a given tame may be shown within summary display 4200, as displayed in FIG. 42.

### Summary Panel

Referring now to FIG. 42, there is shown an example of summary display 4200 which may include summary 4201 of net calories, summary 4202 of sleep, and additional information 4203. This may include, for example a breakdown of summary metric, including net calories for the corresponding time span. Breakdown of intake (consumed), basal, and active calories, including percentages. Pie chart 4204 provides a display of the distribution of the three categories of calories. Sleep summary 4202 displays statistics about user's 100 sleep patterns. In at least one embodiment, the displayed data automatically updates as user 100 scrolls.

Summary display 4200 may provide an aggregated view of metrics displayed in tracks shown on timeline 300. It may respond dynamically to user's 100 interaction with timeline 300, for example, if user 100 moves the playhead along timeline 300. Accordingly, in at least one embodiment, summary display 4200 may display visual playback of timeline 300 data as the playhead moves across timeline 300.

In at least one embodiment, summary display 4200 may summarize and aggregate data from a selected date range on timeline 300, and/or from a specific point in time.

In at least one embodiment, user 100 may customize which data streams are displayed and/or prioritized within summary display 4200.

In at least one embodiment, summary display 4200 may show cumulative/average state for a selected date range. If Today is selected, it may display cumulative/average state until the current time.

In at least one embodiment, if user 100 hovers on the time bar within timeline 300, modules on summary display 4200 may change to a hover state showing additional information.

### Modules

In various embodiments, any number of modules may be included in the application.

### Insights Module

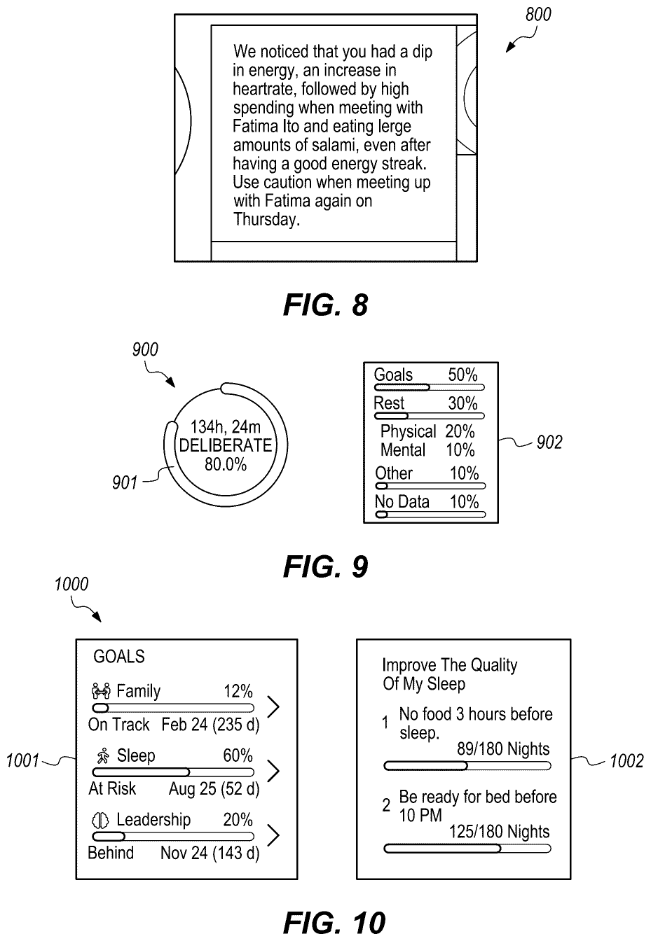

Referring now to FIG. 8, there is shown an example of a display 800 that may be presented by an Insights module. As shown, this module may display Insights as a highlighted textbox so that user 100 can know when a snapshot in time is being annotated by an FDE 251, thus allowing user 100 to reflect on the moment more intentionally. In at least one embodiment, user 100 may access functionality of the Insights module via onscreen windows, cards, or panes that are presented within or adjacent to a view of timeline 300. An example of an Insight is net calories, which may be derived as a synthetic metric by subtracting basal calories and active calories from food calories.

In at least one embodiment, the Insight may be displayed in a box of text that shows approximately the first 250 characters. If the Insight content is longer than 250 characters, then the text may be abbreviated with an ellipsis at the end.

User 100 may click on the Insights module to cause the Insights tab 721 on Side Panel 720 to open to that specific Insight, displaying the related data.

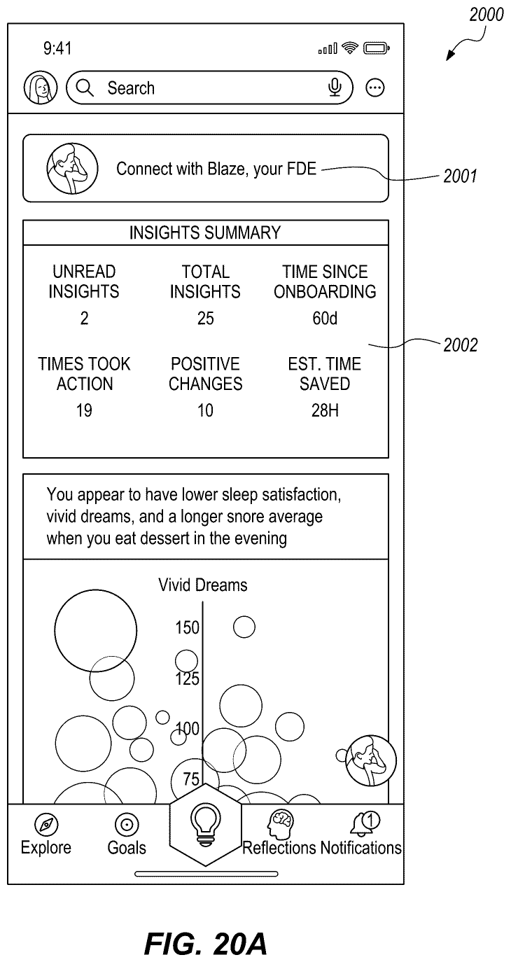

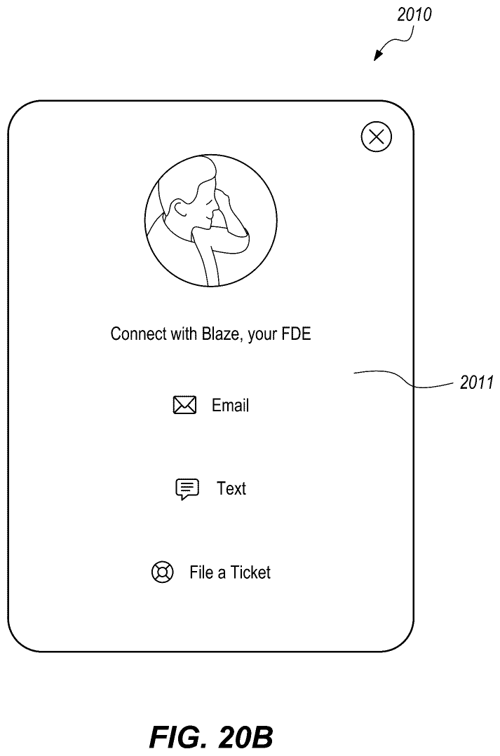

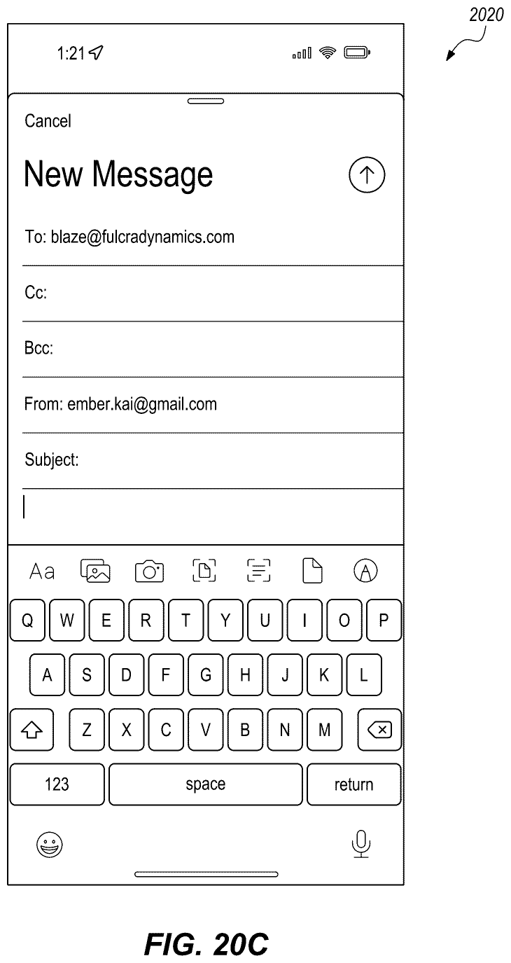

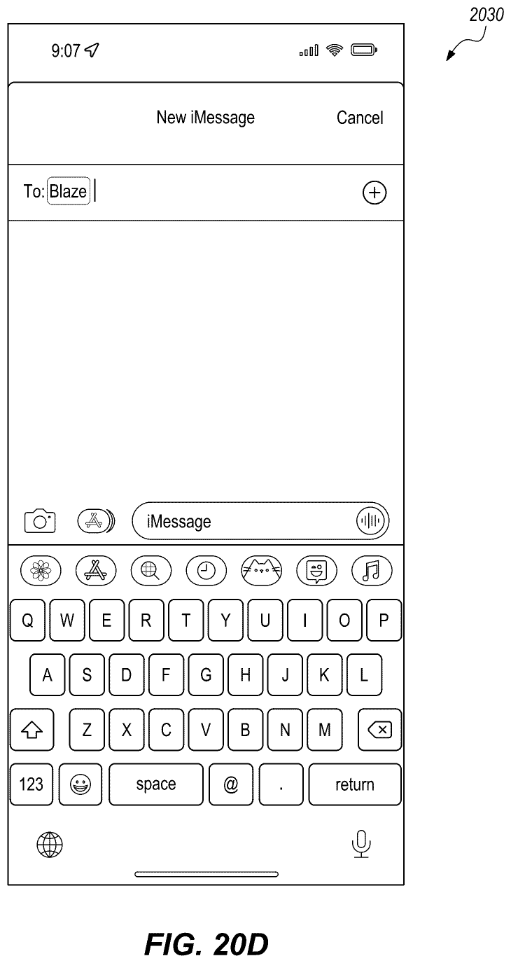

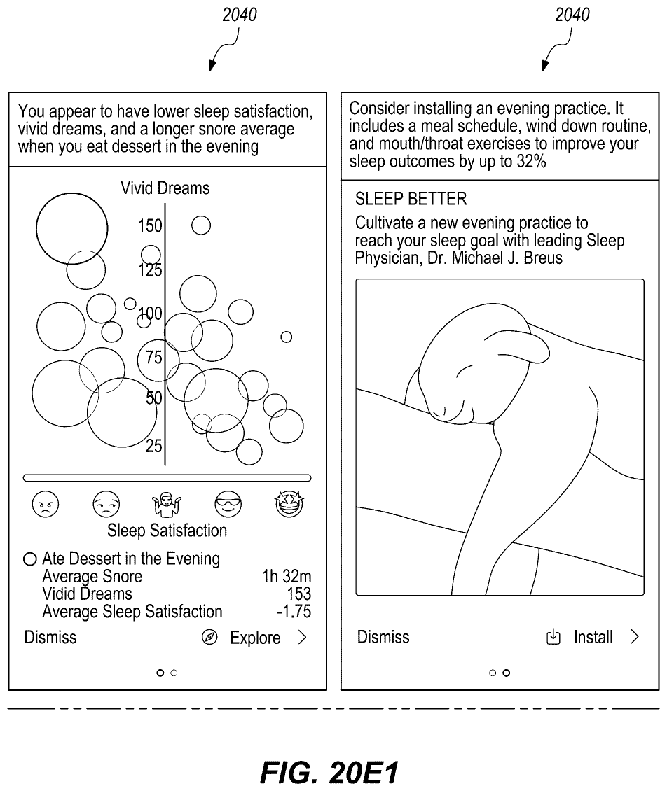

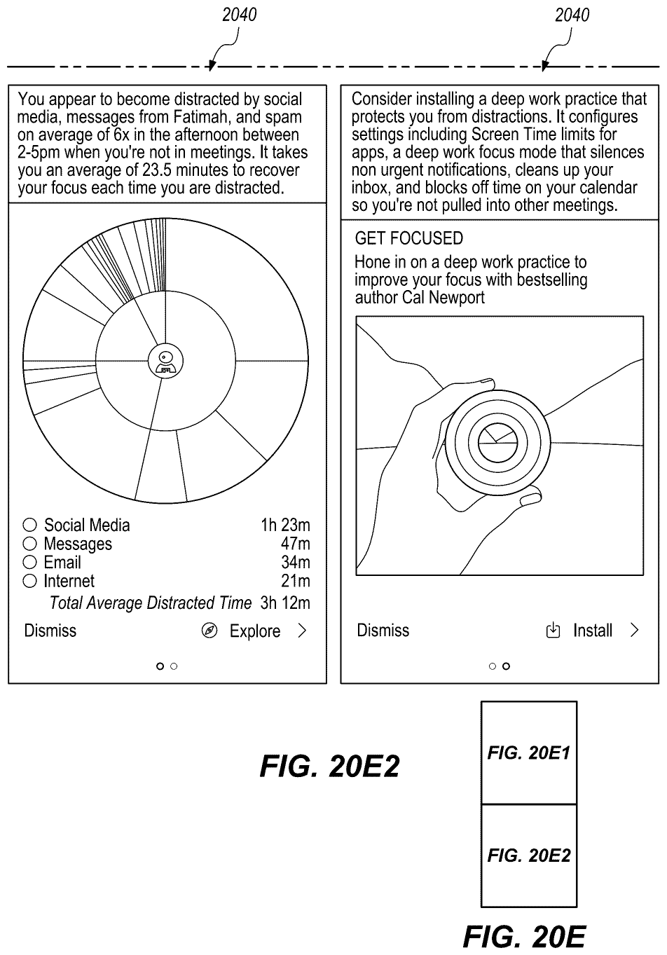

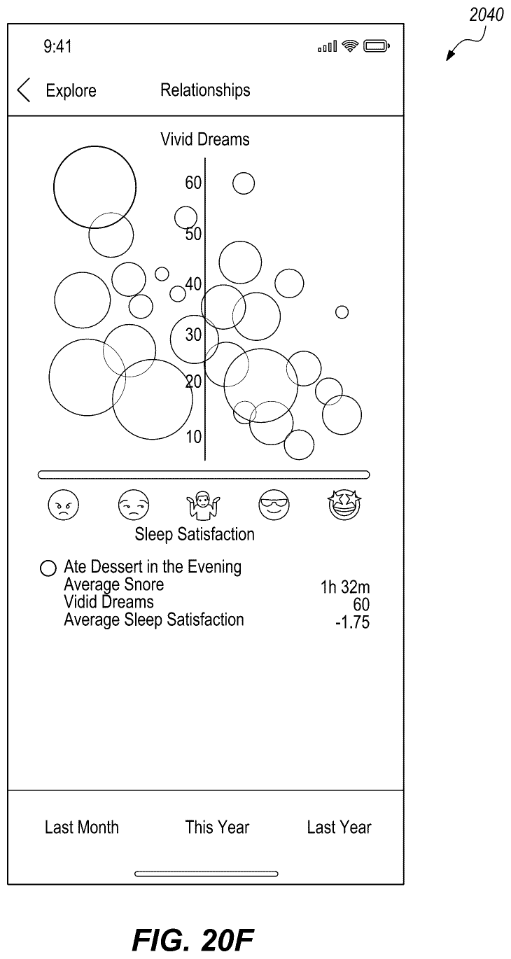

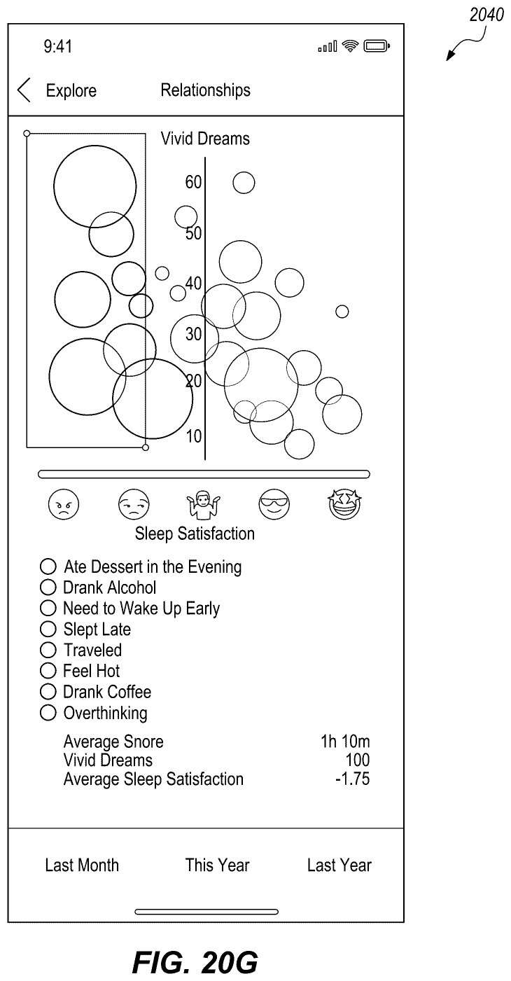

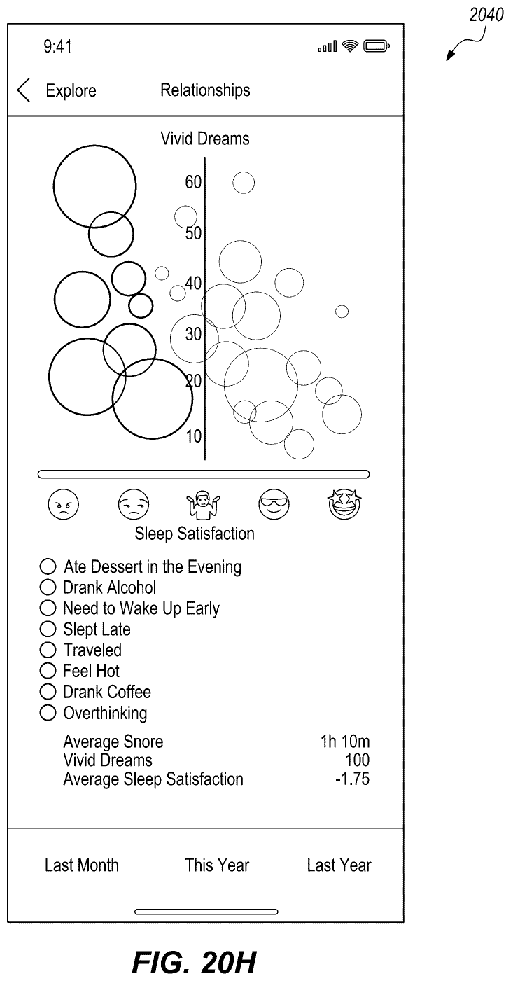

Referring now to FIGS. 20A through 20I, there are shown various insight screens for presenting insights according to one embodiment. In at least one embodiment, Insights is the default tab presented, so as to display the most salient, actionable points for user 100 when the app is activated.

In at least one embodiment, button 2001 may be prominently displayed at the top of each screen, so user 100 knows they can reach out to their FDE 251 if they have any questions.

### Insights Summary 2000

As shown in FIG. 20A, Insights Summary 2000 may be a Key Performance Indicator (KPI) panel that gives user 100 a quick summary of their insights, actions, and outcomes. Such a display may reinforce the action cycle so that user 100 keeps coming to the system for insights that will make a positive impact on their lives. The following information may be provided:
Unread Insights—Makes it easy for user 100 to see when there's something new for them or something they haven't interacted with yet.
Total Insights—Shows how much value the system has delivered to them in the time they have been working with it.
Time Since Onboarding—Makes it easy for users 100 to gauge how long they have been working with the system and the time to value that it has delivered.
Times Took Action—Shows how much effort user 100 has been putting into addressing the insights and recommendations the system has made to them. This number could be the result of installing practices.
Positive Changes—This is a user self-reported number. The system may poll them on occasion about the insights they took action upon and ask for their feedback on whether it resulted in any positive changes.
Estimated Time Saved—As a result of action taken on certain time saving insights such as canceling a meeting, the system can estimate the time saved.
   Connect with FDE 251

FIGS. 20A through 20D depict examples of various screens for contacting FDE 251.

As shown in FIG. 20A, button 2001 to contact FDE 251 may be presented at the top of Insights tab 2002.

In response to user 100 clicking on button 2001, a dialog box may be shown, such as box 2010 depicted in FIG. 20B. A number of contact options 2011 may be presented, such as:
Text: “Connect with [first_name], your FDE
Options: Email; Text
File a Ticket

Clicking on an email contact option 2011 may initiate an email message 2020, with sender and destination pre-filled, as shown in FIG. 20C.

Clicking on a message contact option 2011 may initiate a message 2030, with destination pre-filled, as shown in FIG. 20D.

### Insights With Explore Drill Down and Action to Install

In at least one embodiment, each insight may have two parts:
A finding with a chart providing supporting evidence and a way to explore the data further.
A recommendation with an option to install a new practice to take action upon the finding.

Each insight can be dismissed if user 100 chooses not to take any action. It is then removed from the Insights tab, but can be found in the Insight History at the bottom of the page.

FIGS. 20E through 20I depict examples of various Insights 2040 that may be displayed.

### Metascore Module

Referring now to FIG. 9, there is shown an example of a display 900 that may be presented by a Metascore module. The system may display a metascore that is customized to user's 100 life so that user 100 can track their overall progress as desired, without having to calculate individual metrics.

The metascore reflects user's 100 most important aspects of their life that they want to monitor. An FDE can work with user 100 to customize their score and set up submetrics as desired.

In at least one embodiment, the default view is the “deliberate” metascore donut 901. User 100 may hover over this display to cause the metascore breakdown 902 to be displayed.

In at least one embodiment, a Metascore is calculated by adding the time spent on positive activities user 100 engages in for the date range selected. In at least one embodiment, neutral submetrics are not added in the total percentage. Each submetric is then calculated by adding the time they actually spend on that submetric divided by total amount of time in the date range selected.

In at least one embodiment, each submetric can have tertiary metrics. For example, Recovery may include Physical and Mental Recovery.

In at least one embodiment, the default metascore is “Deliberate”, which may include, for example:
Goals [positive]: Time spent on working towards goals defined in the system.
Recovery [positive]: Time spent on Physical and Mental Recovery.
Other [neutral]: Time spent on activities that were not categorized as a Goal or Rest.
No Data [neutral]: Time that was unaccounted for and could not be categorized because the system did not receive enough information from user 100.

In at least one embodiment, every Metascore may contain neutral submetrics for “Other” and/or “No Data.”

In at least one embodiment, a Deliberateness Score may be calculated. The score may be weighted based on the amount of time an individual spends on the different positive areas of their life, as follows:
Goals: May be calculated in different ways based on what success metrics are defined when the goal is created.
Rest: May be defined as sleep, leisure, or times where an individual is relaxing. This can be determined in any of several different ways, such as for example:
   Mental Rest: Calendar data for scheduled events such as meditation, a scheduled massage, or an outing to go golfing with friends.
   Physical Rest: Heart rate, geolocation data to determine which room of the home an individual is in and determine what they are doing at that time.
   Sleep: May be determined by the sleep analysis identifier on healthkit or the like. A sleep period may be initiated based, for example, on data from the accelerometer and heart rate sensors in a wearable device such as an Apple Watch.
Other: May include areas where the system can determine what an individual is doing. This can be determined in most cases by health data, geolocation, calendar, and/or other sources.
No Data: May include areas where the system cannot determine what an individual is doing based on data sources. In at least one embodiment, the system may stochastically poll an individual in these periods and determine the type of event they are experiencing.

### Goals and Success Metrics Module

Referring now to FIG. 10, there is shown an example of a set of displays 1001, 1002 that may be presented by a Goals and Success Metrics module. Users 100 may wish to see how they are tracking towards their goals so as to understand how they need to adjust their behavior to accomplish goals in a desired timeframe. In at least one embodiment, the system allows users 100 to see their progress on individual key performance indicators that allow them to know when they accomplish their goals. In at least one embodiment, timeline 300 may provide an indication of how much time user 100 spends working towards their goals so it is easier to determine whether user 100 has been spending their time wisely.

Goals may reflect user's 100 most important intentions structured with milestones, ways to measure progress, and a deadline. An FDE 251 may work with user 100 on customizing their goals and getting the success metrics instrumented.

Ideally as a lifestyle designer works with user 100 to set goals, the goals may be rooted with consideration of the performance indicators that can be utilized to track success. User 100 may want to track a variety of different goals, some of which have success metrics. Examples include:
Increase sleep quality:
   No food 3 hours before sleep—Glucose monitor.
   Winding down for bed before 10 pm—Heart rate and sleep analysis.
   Wake up at 6 am regularly—Heart Rate and sleep analysis.
Reduce stress:
   Meditate 3 times a week for at least 20 minutes—Stochastic poll, heart rate, and calendar.
   Go for at least one walk per week—Step Count, heart rate, and geolocation.
   Spend more time with my partner—local network connections, geolocation data.

Each of these goals may be tracked using various data sources associated with user 100. Each goal may have different requirements and success metrics that are personal to user 100. The hobbies or interests of some will not apply to all. In at least one embodiment, the system takes into account each user's 100 individual preferences and uses those to inform what habits play into their use of time.

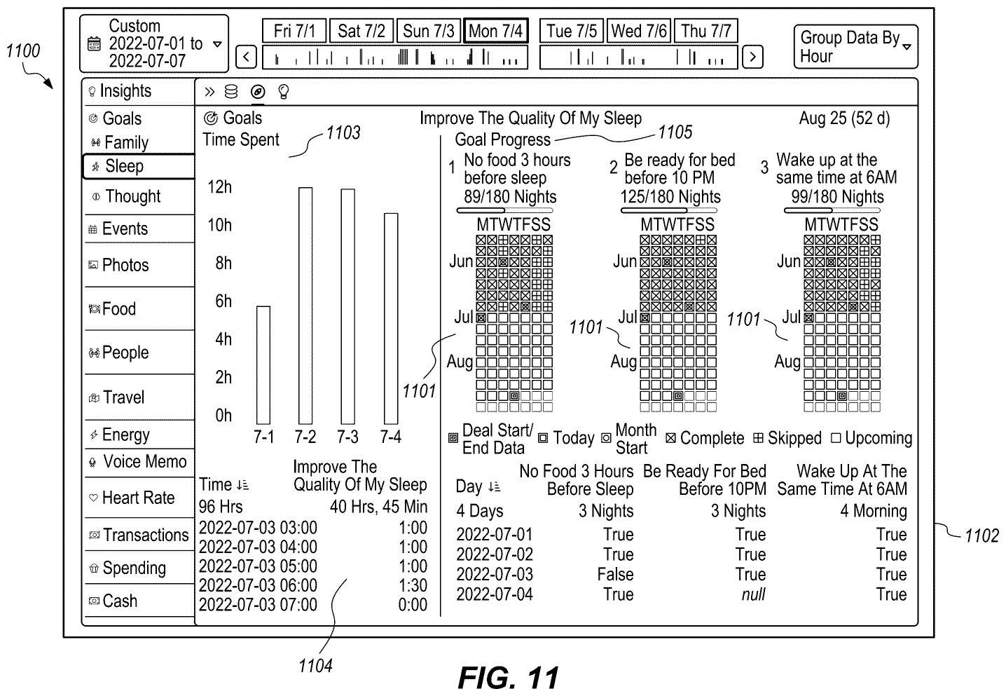

Referring now to FIG. 11, there is shown an example of a display 1100 that may be presented by a Visualization module in connection with goals and success metrics.

In at least one embodiment, a default view on visualization display 1100 may show goal progress. The top three goals for user 100 may be presented. If there are more than three goals, user 100 can scroll to see the rest of the list.

In response to user 100 clicking on a goal or detail icon, an overlay with the progress on success metrics for that specific goal may be displayed. User 100 can click outside of the overlay to dismiss it. The icon may represent which category the goal is related to. The goal label may be a single word, which is a shortened version of the complete goal. The complete goal text may be displayed at the top of the details overlay.

Date detail 1102 on the bottom right shows the month and day the goal is targeted for with the day countdown in parentheses.

The color of the progress bar, progress percentage, and contextual progress label may change color depending on progress status, as follows:
Green (success): On Track
Orange (warning): Behind
Red (danger): At Risk

In at least one embodiment, a progress percentage is calculated by determining the actual progress for all success metrics divided by the total goal amount, then multiplied by 100. Progress status may be determined manually by user 100 periodically reviewing their goals and reflecting on their progress.

Time Spent. Total time spent per day on the goal may be displayed in a vertical bar chart 1103. A table 1104 may be presented that shows the timestamp and aggregated elapsed time value for each row depending on granularity set (e.g., hour, day).

The default sort on the table may be newest to oldest, although in other embodiments the data may be sorted in a different manner.

The Time column's total row shows the total elapsed time for the selected date range. The Goal column's total row shows the total time spent on that goal for the selected date range.

Goal Progress (Success Metrics). Charts 1105 displayed for each success metric may vary depending on what FDE 251 sees fit for representing the data. In the depicted example, the metrics are best suited to being tracked at the day granularity.

The default chart may be a temporal waffle chart 1101 meant for binary data input (i.e., True∥False∥null). The chart header shows the overall progress made towards that success metric. The color of the success metric text matches the text of the corresponding table header. Each block in chart 1101 represents a day.

In at least one embodiment, there may be five different indicators to describe that day:
Goal start/end date: red outline
Today: purple outline
Month start: white outline
Complete: mint green
Skipped: grey
Upcoming day: white

Everything else may be shown in dark grey for the remaining part of the month that is not planned for the goal.

In at least one embodiment, the chart scrolls vertically to show the rest of the time in the goal date span. The default scroll position is pinned to today as close to the top of the chart as possible. If there are more than three charts 1101, user 100 is able to scroll horizontally to view the rest.

Table 1102 shows the timestamp and binary data, with a default sort from newest to oldest. The Day column's total row shows the total number of elapsed days for the selected date range. The success metric column's total rows show the total number of days where the value is True.

### Events Module

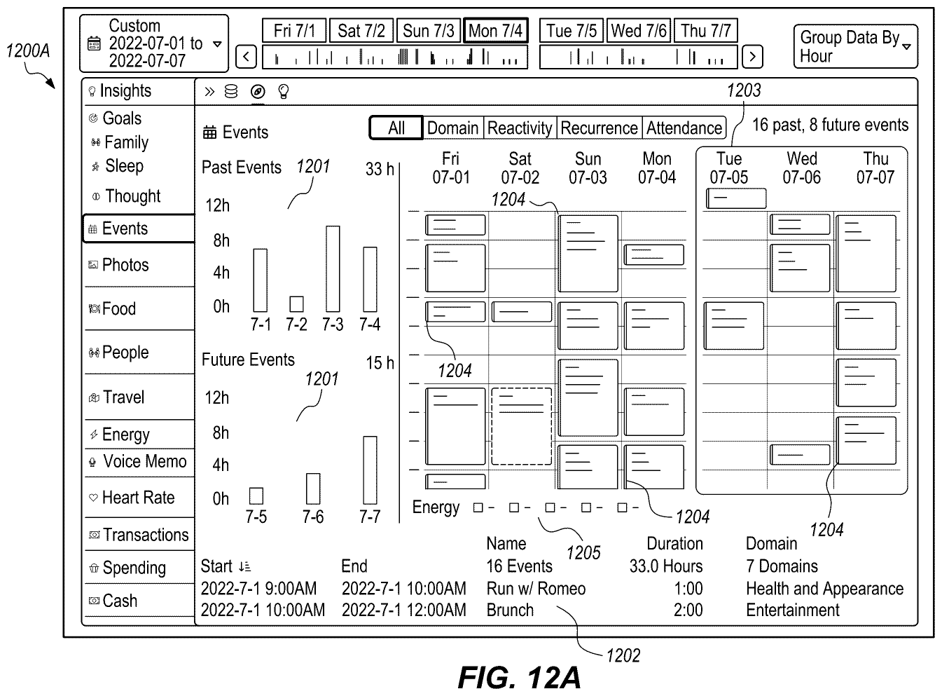

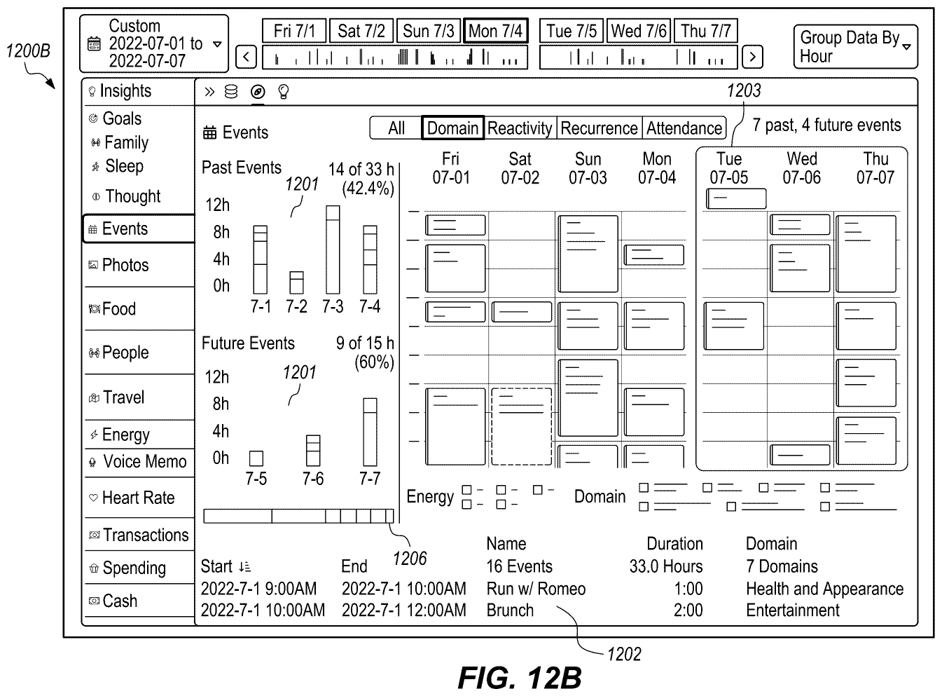

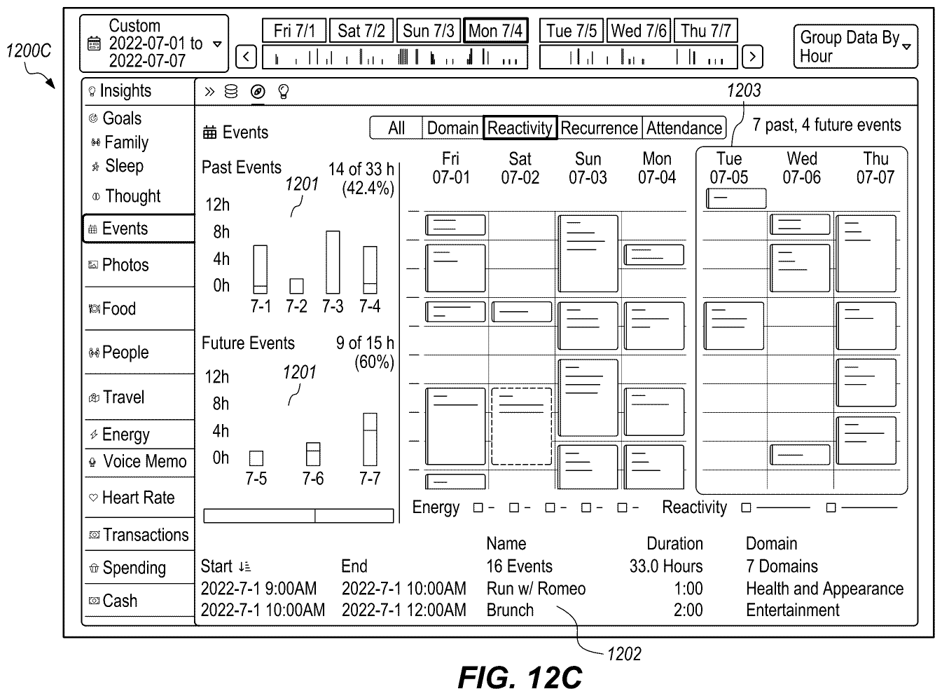

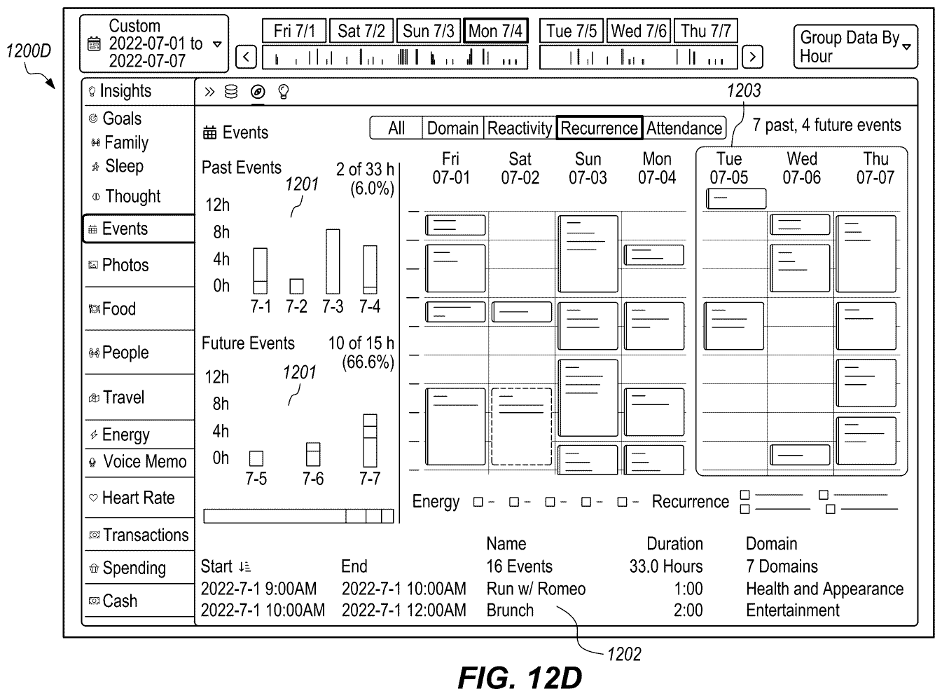

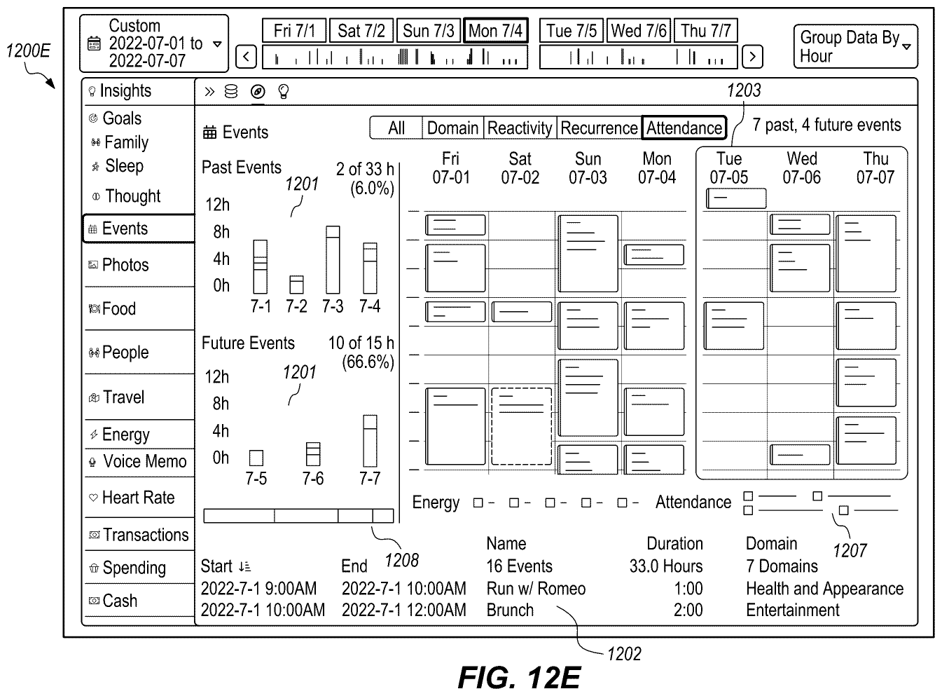

Referring now to FIGS. 12A through 12E, there are shown examples of a display 1200 that may be presented by an Events module that can help user 100 understand how different event characteristics affect their quality of life so that they can decide which events they want to continue putting energy towards.

The left-hand side of each display 1200 includes two charts 1201 including duration by day. The right-hand side shows a calendar view 1203 with the events. Both are split into past and future events.

Data Table 1202. Table 1202 shows data for past events, as follows:
Start—timestamp of when event started
End—timestamp of when event ended
Name [#Events]—title of the event
Duration [#h, #m]—total elapsed time of the events
Domain [#Domains]—total number of unique domains
Reactivity [#.#% Reactive]—average number of reactive events
Recurrence [#.#% Recurring]—average number of recurring events
Attendance [#People]—total number of unique people
Energy [+/−#.#]—average energy value across events

Energy Levels 1204. In at least one embodiment, each event can have an energy level 1204 associated with it as indicated by the colored bar on the left of the event block. If no energy level is recorded for the event, then the left-hand bar is outlined. In at least one embodiment, user 100 can click an unscheduled event or an event without a recorded energy level 1204, and is prompted to input their energy level. Legend 1205 shows the meaning of the various colors for energy levels 1204.

Presets. In at least one embodiment, presets may be provided, for displaying different sets of metrics for different purposes. For example, a trainer view that shows metrics they care about, an assistant view that shows calendar and location and sleep, and/or the like.

Filters. In at least one embodiment, filters may be applied to the charts and calendar. Examples of such filters include:
All—Every event within the selected date range
Domain—How user 100 categorized the event type
Reactivity—Whether the event was proactively scheduled by user 100 or the event was placed on their calendar
Recurrence—The periodicity of each recurring event
Attendance—Number of people per event

FIG. 12A depicts an All display 1200A, including all events that are in user's 100 calendar in one place, unfiltered, allowing user 100 to quickly review past and upcoming events. Every event within the selected date range is displayed without filtering. Past Events and Future Events charts show total event duration by day.

FIG. 12B depicts a Domain display 1200B including user's 100 events categorized by Domain, allowing user 100 to understand which areas of their life they spend the most time on and how each domain affects their energy levels.

Events may be color-coded by Domain. Past and Future Events charts may include bar segmented by Domain and stacked vertically by longest duration on the bottom. Horizontal category bar chart 1206 may be proportioned to 100% of events by Domain with the longest duration segments stacked to the left. In at least one embodiment, users 100 can create their own Domain labels and assign custom colors.

FIG. 12C depicts a Reactivity display 1200C including user's 100 events to which user 100 is reactive and proactive, so as to allow user 100 to be more proactive with their event acceptances and understand the impact of events to which they are being reactive in relation to their energy levels.

Events may be color-coded by reactivity. Events where user 100 reacted to an external event request may be marked as Reactive.

Events where user 100 created the event request are marked as Proactive. The Past and Future Events charts may have bars segmented by Reactive and Proactive and stacked by longest duration on the bottom. The horizontal category bar chart may be proportioned to 100% of events by reactivity with the longest duration segment to the left.

FIG. 12D depicts a Recurrence display 1200D including user's 100 events categorized by how frequently they recur, so as to allow user 100 to understand the long-term impact of events that recur very frequently in relation to their energy levels.

Events may be color-coded by recurrence. Events that are set to recur are labeled according to their periodicity. For example:
Daily
Weekly
Bi-Weekly
Monthly
Quarterly
One-Time

In at least one embodiment, recurrence categories may only be displayed in the legend if they are present in the data. Events that do not recur may be labeled as One-Time events. Past and Future Events charts 1201 may have bars segmented by their recurrence type and stacked vertically by longest duration on the bottom. The horizontal category bar chart may be proportioned to 100% of events by recurrence type with the longest duration segment to the left.

FIG. 12E depicts an Attendance display 1200E including user's 100 events categorized by the number of people attending the event, so as to help user 100 understand the impact that people have on their energy and how effective they can be with their time in relation to other peoples' time.

Events may be color-coded by attendance. Events may be labeled according to the number of people listed on the event. For example:
Solo
1:1
3+ People
10+ People

In at least one embodiment, attendance categories may only be displayed in legend 1207 if they are present in the data. Past and Future Events charts 1201 may have bars segmented by their attendance type and stacked vertically by longest duration on the bottom. Horizontal category bar chart 1208 may be proportioned to 100% of events by attendance type with the longest duration segment to the left.

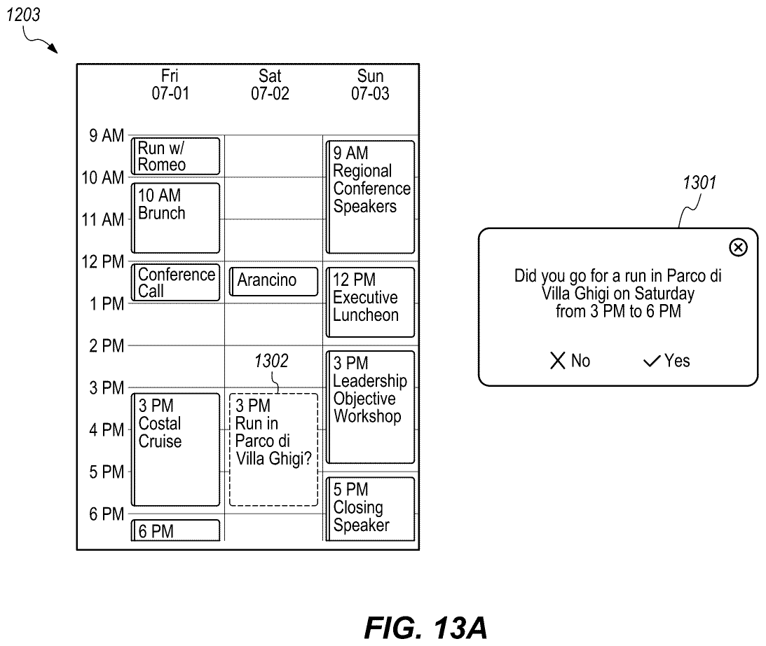

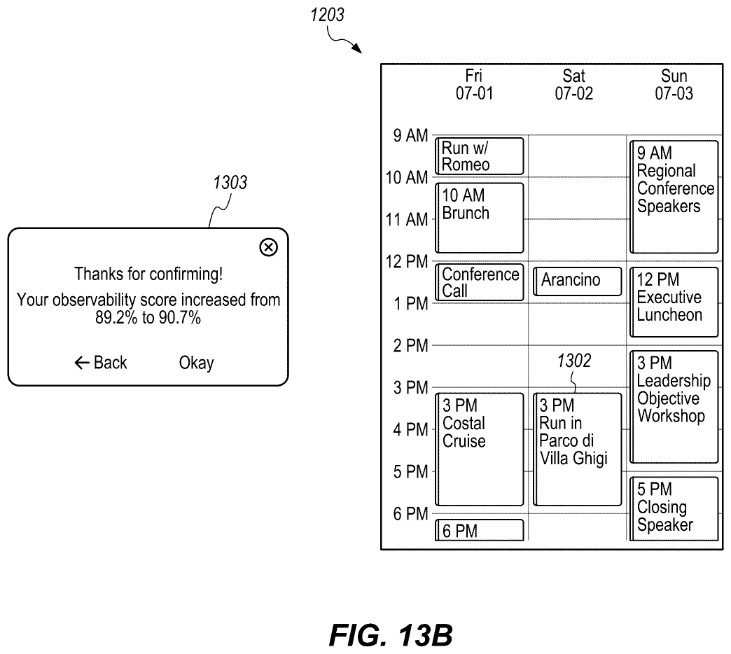

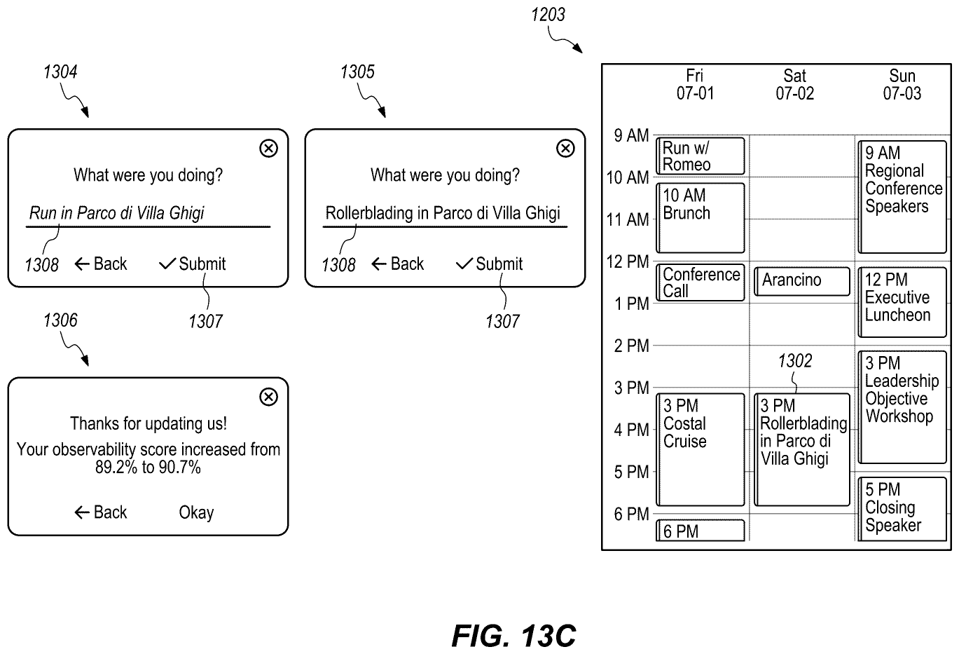

Referring now to FIGS. 13A through 13C, there are shown examples of displays for Unscheduled Event Detection. In at least one embodiment, the system can provide user 100 with recommendations for unscheduled events that the system detects based on user's 100 activity, so that user 100 can increase the accuracy and observability of their data.

FIG. 13A depicts an example of a prompt 1301 that may be displayed in response to user 100 clicking on an unscheduled event 1302 highlighted within calendar view 1203 in purple. User 100 may be prompted to confirm whether the detected event is accurate (Yes) or not (No). The text of the prompt may be, for example, “Did you attend [detected event title] on [Day] from [#AM/PM to #AM/PM]?”

As shown in FIG. 13B, in response to user 100 clicking on “Yes,” the system may acknowledge the confirmation, by displaying message 1303, which may state, for example, “Thanks for Confirming.” An observability score update may be display, such as for example: “Your observability score increased from #.#% to #.#%”. Once user 100 clicks on “Okay” to complete the confirmation, the system updates event 1302 within calendar view 1203.

As shown in FIG. 13C, in response to user 100 clicking on “No,” the system may display prompt 1304 to ask user 100 to input the correct event title, for example by displaying, “What were you doing?” In at least one embodiment, as shown in dialog box 1305, the system may display textbox 1308 with the default text of the original recommendation as a reminder, and may display confirmation message 1306 once the user responds.

In at least one embodiment, Submit button 1307 may be disabled until text is inputted into textbox 1308. Clicking on textbox 1308 clears the text to make it ready for input. In response to user 100 clicking on Submit button 1307, the system may acknowledge the updated event title, for example by displaying confirmation message 1306, “Thanks for updating us!” An observability score update may be displayed, such as for example: “Your observability score increased from #.#% to #.#%”. Once user 100 clicks on “Okay” to complete the confirmation, the system may automatically update event 1302 on calendar 1203.

### Signal Strength Module

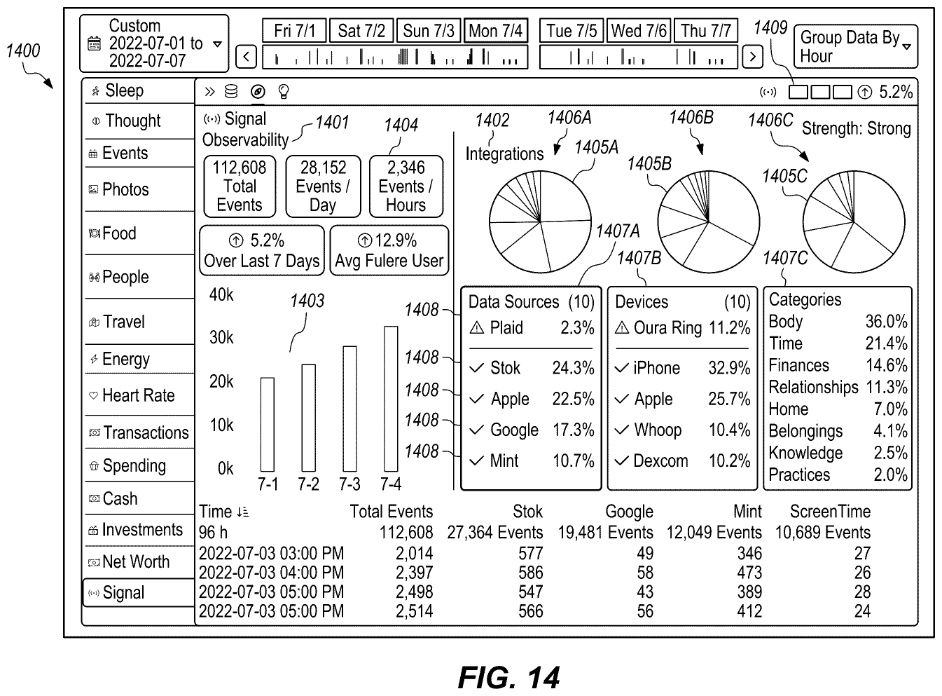

Referring now to FIG. 14, there is shown an example of a display 1400 for a Signal Strength module that can help user 100 understand how well their life is instrumented for sending the system their data so that they can increase their levels of observability and integrations, which will enable the system to make the highest quality assessments.

In at least one embodiment, user 100 may access the Signal Strength module via a Home view of a Portal, which may show trends in how many records are being collected.

In at least one embodiment, Signal Strength display 1400 includes display 1401 of Observability metrics and display 1402 of Integration metrics. The goal of the Signal Strength module is to provide feedback to user 100 on how much the system is able to observe, while incentivizing them to provide more data without “punishing” them for not integrating with new data sources or devices as the system's ability to integrate increases.

A Signal Strength Metric System may be established. For every Explore drill down, signal strength indicator 1409 may be shown. The three bars represent the strength level, as follows:

| Strength | Bars | Color | Calculation |
|---|---|---|---|
| None | 0 | Gray with Red X | No data |
| Weak | 1 | Red | 25th percentile of events/hour |
| | | | across all team members |
| Moderate | 2 | Orange | 50th percentile of events/hour |
| | | | across all team members |
| Strong | 3 | Green | 75th percentile of events/hour |
| | | | across all team members |

Observability describes how many events the system was able to observe from user's 100 DataStream. The top Key Performance Indicators (KPIs) 1404 show the events breakdown and comparisons, as follows:

| Metric | Purpose | Calculation |
|---|---|---|
| Total | Get user 100 | Sum of all events for the selected |
| Events | thinking about the | time period that has occurred. |
| Events/ | sheer volume of | Total Events divided by the number |
| Day | events at an | of days in the selected time period |
| | impressive rate | that has occurred. |
| Events/ | they are able to | Total Events divided by the number |
| Hour | generate and send | of hours in the selected time period |
| | to the system to | that has occurred. |
| | observe for them. | |
| Percent | Show user 100 | Total Events for last full day minus |
| Change | their individual | Total Events for 7 days prior; divided |
| Over | progress towards | by Total Events for 7 days prior; |
| Last 7 | improving their | multiplied by 100 |
| Days | observability by | |
| | displaying how | |
| | much their event | |
| | rate has changed | |
| | over the last 7 | |
| | days. | |
| Percent | Show user 100 | The percent more/less than the |
| Difference | how they compare | average team member's event/hour |
| from | to others in their | rate. |
| Average | cohort by | |
| Team | displaying how | |
| Member | much their event | |
| | rate differs from | |
| | the average event | |
| | rate across team | |
| | members. | |

Bar graph 1403 may show the volume of Total Events by day. In response to user 100 hovering over this display, the value of Total Events may be displayed in a tooltip.

Integrations. Integrations section 1402 may show user 100 three different breakdowns 1406A, 1406B, 1406C of user's 100 integrations:
Data Sources 1406A—software that collects/handles/generates data.
Devices 1406B—hardware that is capable of data input or generation.
Categories 1406C—main categories of life that the system defines.

Observability metrics may be displayed in order to help user 100 to understand the scale of and completeness of observability over their life, so that they can feel assured that as much of their life is being preserved as possible. The goal is to help user 100 understand:
when data is being captured or not;
how much data is being captured;
whether the data flow is increasing or decreasing compared to the average amount; and
how much data has been collected overall.

The types of aggregated observability metrics presented to user 100 may include:
All Events: an overview of the entire corpus of data they are having Fulcra preserve with a feel for the scale and completeness of the observability of their life.
Manual Inputs: the physical and digital button presses captured via the API. In at least one embodiment, manual inputs may include ad hoc events user 100 manually documents.
Calendar: measurements derived from user's 100 calendar(s) at the calendar invite level and the hour level for cumulative time observed from calendar invites.
Location: measurements derived from user's 100 physical location via their phone or other tracking device. There may be more than one level of granularity including discrete locations identified based on groupings of location measurements using a threshold and all location measurements collected.
Health: all health measurements derived from health applications.
Sleep: all sleep measurements derived from health applications.
Integrations: the observability metrics for the sources (e.g., apps, APIs, libraries) and devices (e.g., wearables, physical sensors) that generate, capture, or enrich user data. Each subsection (i.e., Data Sources and Devices) may have the count of items X in the top right hand corner (X).

Total and trend for selected date may allow user 100 to evaluate whether data is coming in and how much on a daily basis, as well as whether the volume is increasing or decreasing compared to the previous seven days.

Trends may be represented with the following visual distinctions:
Positive trend may be displayed in success green with a circle-arrow-up icon.
Negative trend under 20% may be displayed in warning orange with a circle-arrow-down icon.
Negative trend over 20% may be displayed in danger red with a circle-arrow-down icon.

Cumulative total metrics may give user 100 a sense of the scale at which the system is observing and preserving their data. They may also convey the completeness of observability that the system has of their life in each category.

Averages by time ranges may give user 100 something to compare the selected date with to understand if there is significant increase or decrease in observability and whether troubleshooting is necessary. The granularity of the averages may depend on the volume/scale of the data.

The display may include pie charts 1405A, 1405B, 1405C. Labels may be displayed on the largest slices.

User 100 may hover over smaller slices of the pie charts 1405A, 1405B, 1405C, causing the name to be displayed in a tooltip. In at least one embodiment, a color map such as the Viridis color map can be used for generating sequential color scales.

The display may include lists 1407A, 1407B, 1407C. A count of data sources and devices may be included in parenthesis. Icons 1408 may be included to indicate integration status, as follows:
Checkmark—Connected
Triangle Exclamation—Issue with connection
X—Not integrated

Issues may be pinned to the top of the list.

The time data was last received may be shown in a format such as: [Today∥Yesterday∥#day(s) ago] [at #: ##AM/PM]. The percentage is how much of the data originated from that data source/device or falls into a particular category.

For the Data Sources and Devices lists, if there is an issue with any of the items, it may be placed at the top of the list. In the depicted example, Plaid and Oura Ring 3 have warnings because the last time the system received data was 4 days and 2 days ago respectively. Any suitable threshold may be used for activating this warning, such as for example failure to receive data for 24 hours.

As shown in the example, the data table may adapt based on which chart is selected (i.e., Data Sources, Devices, Categories). Elements of this display may have the following characteristics:
Time—timestamp grouped by hour
Total Events—number of all the records collected during that hour
Metrics—values of individual data sources, devices, or categories

### Receiver API 1702

As described above in connection with FIG. 17, Receiver API 1702 is a component that ingests client data from Context App 1501 and/or other sources. It may be written, for example, in Python & FastAPI and deployed onto Google Cloud Run.

Client data sent to the service may be stored in a data store 106 such as Google Cloud Storage and/or any local or remote file system) for later processing by Loaders.

All incoming requests to this service may be sent through a public GCP load balancer infrastructure and may be authenticated via GCP IAP or API keys.

Receiver API 1702 may be built and containerized via a Google Cloud Build pipelines, and published to a Google Artifact Registry. New builds may be automatically triggered by Github release tags and tagged with the corresponding release tag.

Production deployment may be part of the same Google Cloud Build pipeline trigger that builds and publishes the receiver container image.

Alternatively, a receiver container image can be manually deployed to the GCP Cloud Run service using the gcloud run deploy command.

### Loaders 1714

As described above in connection with FIG. 17, loaders 1714 may be implemented as Celery task workers responsible for retrieving client data from data store 106, processing it, and processing and transforming received source data into SQL tables for storage in database 1715. In at least one embodiment, loaders 1714 may be deployed onto GCP Virtual Machines (VMs).

The primary functionality of loaders 1714 is to retrieve client data from data store 106, process it, and load it into database 1715, which may be implemented as a PostgreSQL database.

Functionality may include for example:
loaders.tasks.process_insert: Load a batch data upload from data store 106, process each file, and load them into database 1715.
loaders.tasks.process_file: Load a single uploaded data file from data store 106, process, and load into database 1715.

### Re-Queuing Stale Batch Uploads for Processing

In at least one embodiment, utility scripts may re-enqueue individual batch uploads or all batch uploads still left on a data store are included in the loader-workers container image. These can be easily run on a live system by docker exec'ing into a running loaders container.

To re-enqueue a specific batch upload, the system may use the utils.enq script:
$ docker exec-ti composer_loader-workers_1/bin/bash
#python3-m utils.enq<account id><batch id>

To re-enqueue any batch uploads left in the data store the system may use the utils.walk script:
$ docker exec-ti composer_loader-workers_1/bin/bash
#python3-m utils.walk

If run within a running container, both scripts may already contain all run-time information necessary for data store and celery configuration.

### Observability

In at least one embodiment, loaders logs may be shipped to the Google Cloud and made accessible via the Google Cloud logging console. Task output may be structured and filtered by celery task name and id, user id, and/or the like.

### Explore Tab

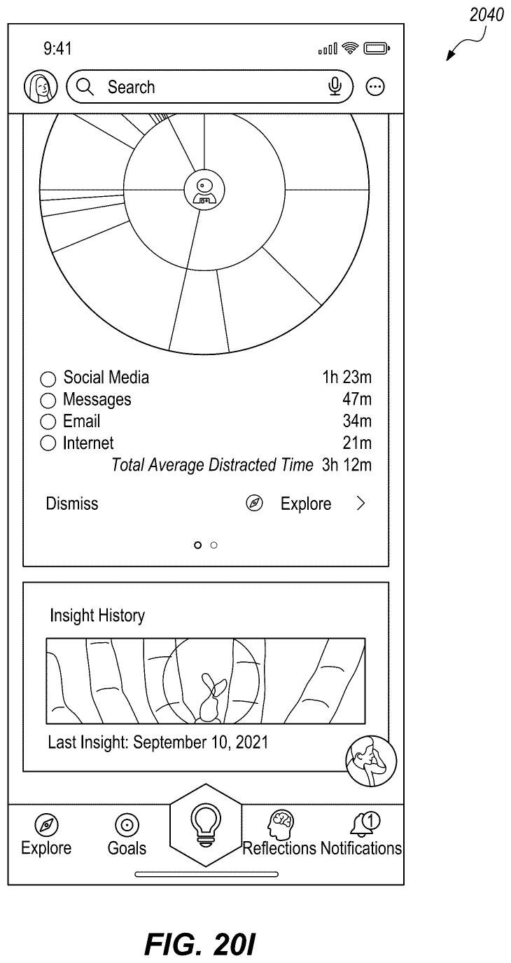

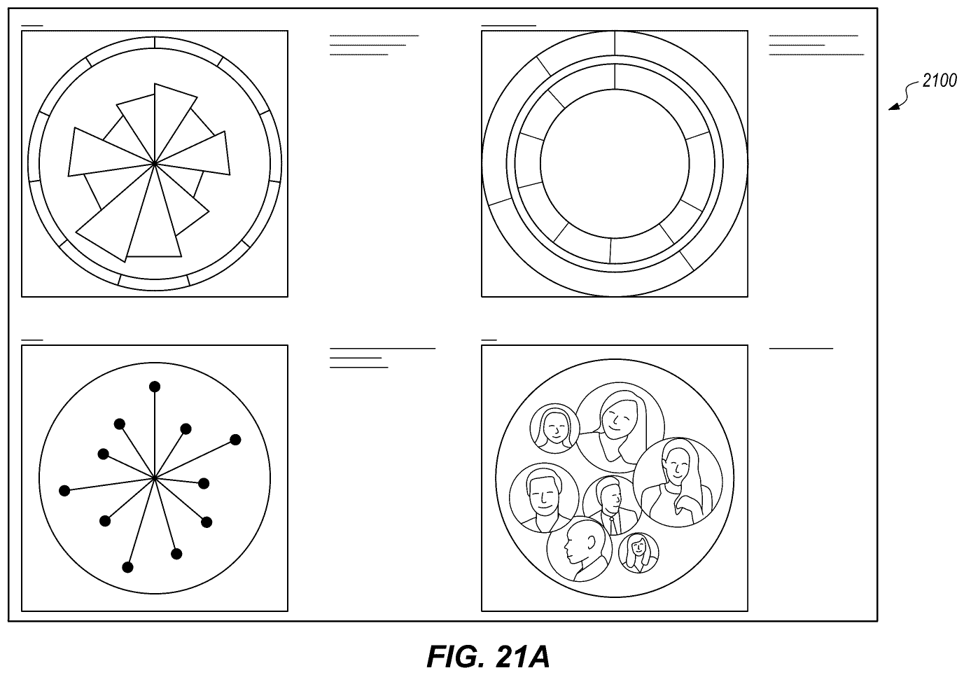

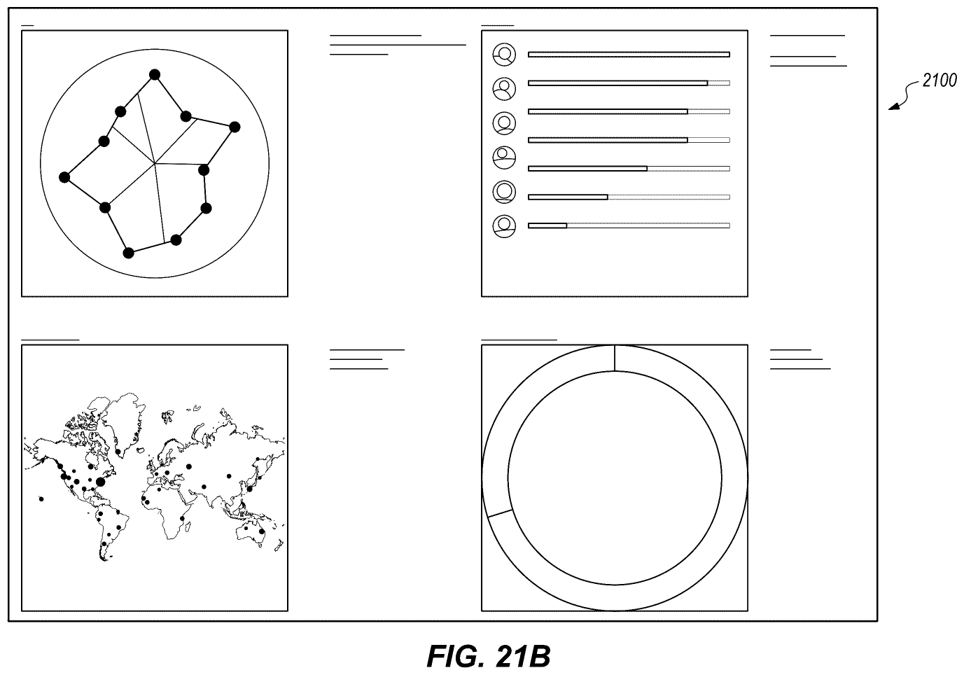

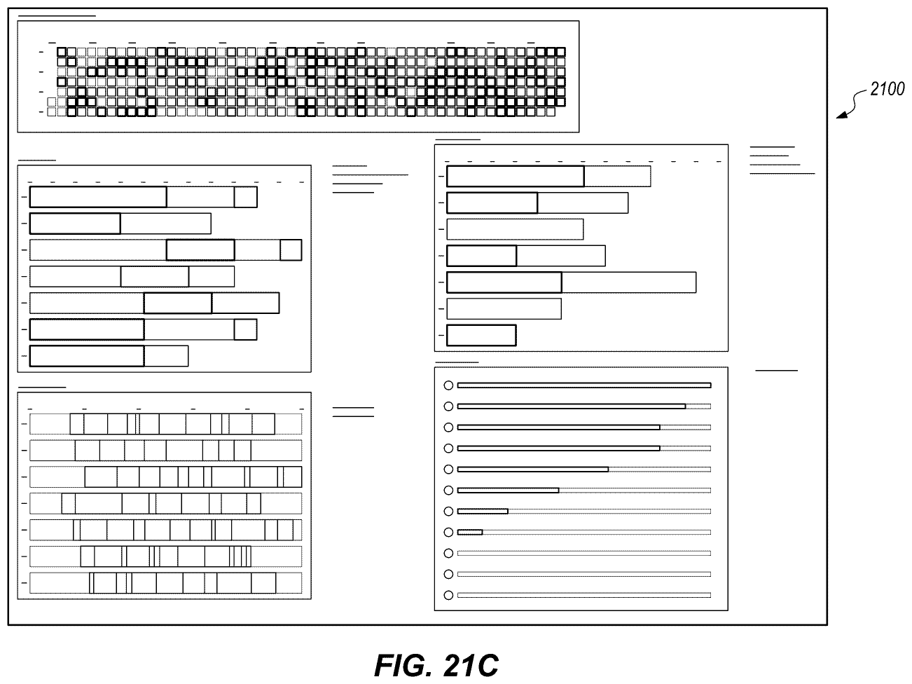

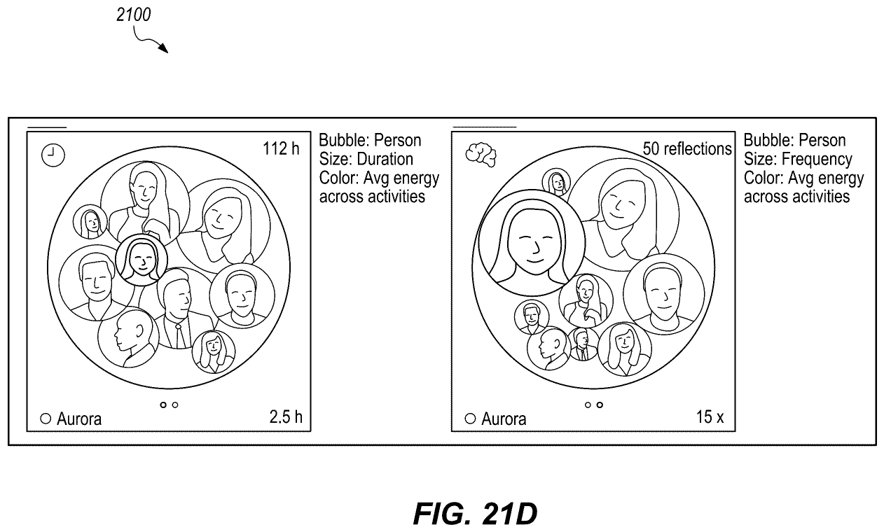

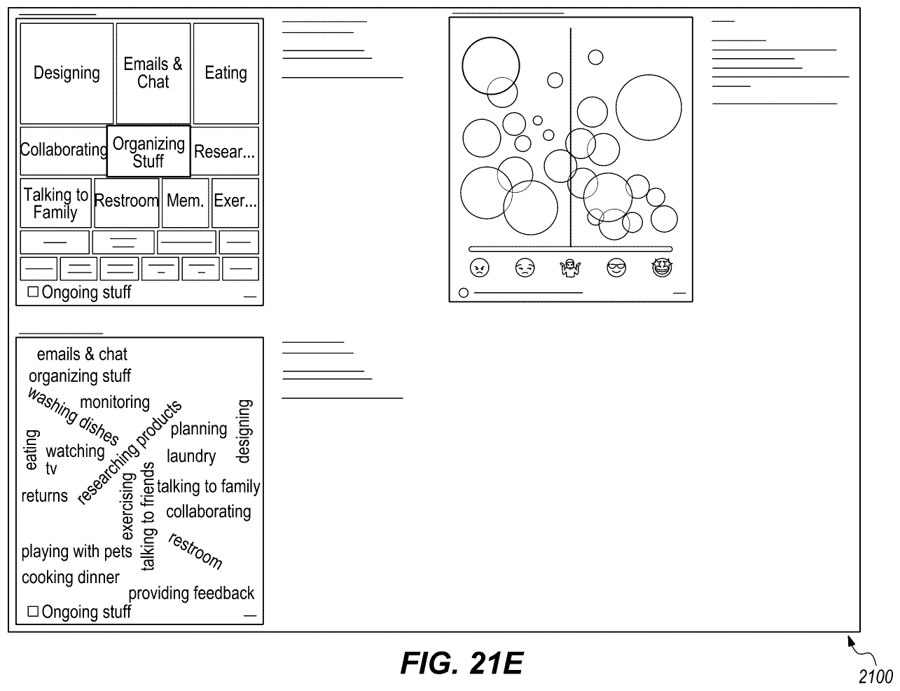

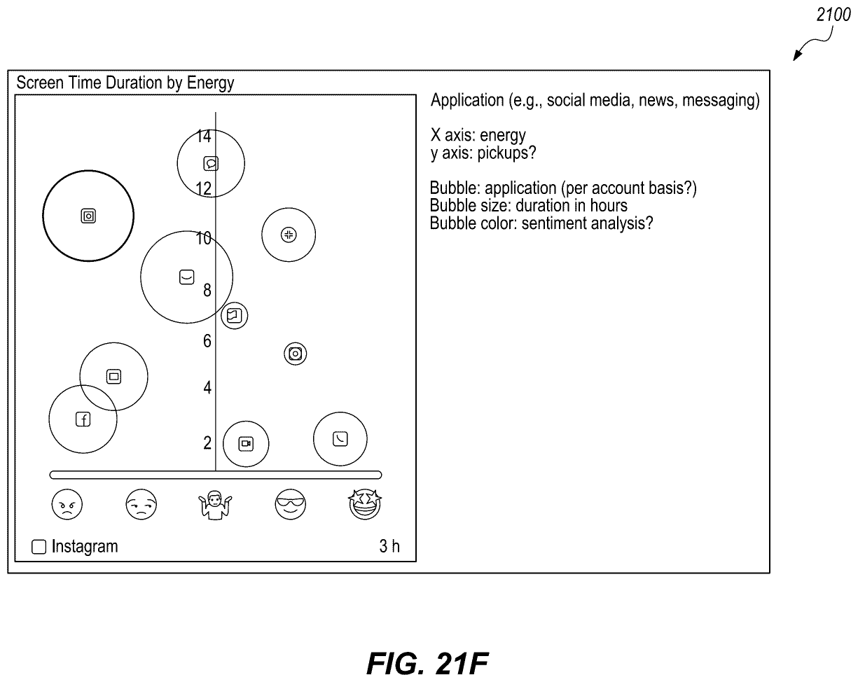

Referring now to FIGS. 21A through 21F, there are shown examples of various visualizations 2100 that may be available via an Explore Tab, according to one embodiment. The Explore Tab may provide a visual summary of user's 100 data. Such visualizations may include, for example:
Centerpiece Viz: May be constructed as a Line Dot chart that uses distance from the center to indicate time spent, line color to indicate average energy, and a dot at the end to indicate the category. Examples include category, time, and energy as the main components.
Social Metrics & Meetings: May include indications of who user 100 has spent time with, and/or energy co-occurrences and category breakdowns. Activities can be broken down as well, such as for example recurring vs. ad hoc; attendance, and/or the like. Also included may be a comparison between time spent with individuals and number of times thinking about individuals (i.e., “who are you thinking about?”).
In some cases, it may be beneficial to include user 100 on the displayed charts. The system can provide output including visualizations 2100 of individual work and/or self-care on the time spent chart and self-reflection on the times in user's 100 thoughts chart. Comparing individually focused time in relation to outwardly facing social relationships can help user 100 determine if they want to protect more time to themselves or spend more time with the people closest to them. In at least one embodiment, the system can potentially determine these intentions from the activities user 100 completes alone. For example:
   Time spent chart. This may include:
      Individual work: Users' 100 time spent doing a work category activity without another person. This can be broken down into project (in service of career, team, personal interests) and maintenance (in service of family unit, home, etc.) related activities.
      Self-care: Users' 100 time spent doing something to improve their health and well-being.
      Time in your thoughts chart. This may include self-reflection: number of times user 100 reflects without someone in mind, and is more inwardly focused.
The display may include Screen Time. For example, the system may use a Screen Time feature to triangulate data about what user 100 is engaged in when they're not doing a calendar tracked activity. This Screen Time data may be paired with co-occurrence data from ad hoc reflections, such as energy. The display may include the following characteristics:
   Bubble=application.\
   Bubble size=duration in hours.
   Bubble color: sentiment analysis
   X-axis: energy
   Y-axis: number of pickups, which may be translated as the level of distractibility this app has on user 100.
The display may include Location.
The display may include Temporal information, such as for example categories, energy, and/or the like in temporal breakdowns.
The display may include Activity. Any of a number of different mechanisms can be used for showing activity breakdowns depicting the areas of user's 100 life their attention is going towards. Examples include:
   Tree Map: This may include the proportion each activity takes across a time span, along with a display depicting how user's 100 energy is distributed across activities.
   Word Cloud: May show activities that are on the ends of the spectrum.
   Bubble Chart: Each bubble may represent an activity. The size of each bubble may represent the duration per instance of the activity. The y-axis represents the frequency of that activity (i.e., recurs). The x-axis represents the average energy for each activity. Optimizations may include:
      Making the activities that bring them higher energy to occur at a higher frequency and/or longer duration.
      Making the activities that bring them lower energy to occur at a lower frequency and/or shorter duration
      Getting all of the activities on the chart to be on the right-hand side.

### Notifications Tab 1615

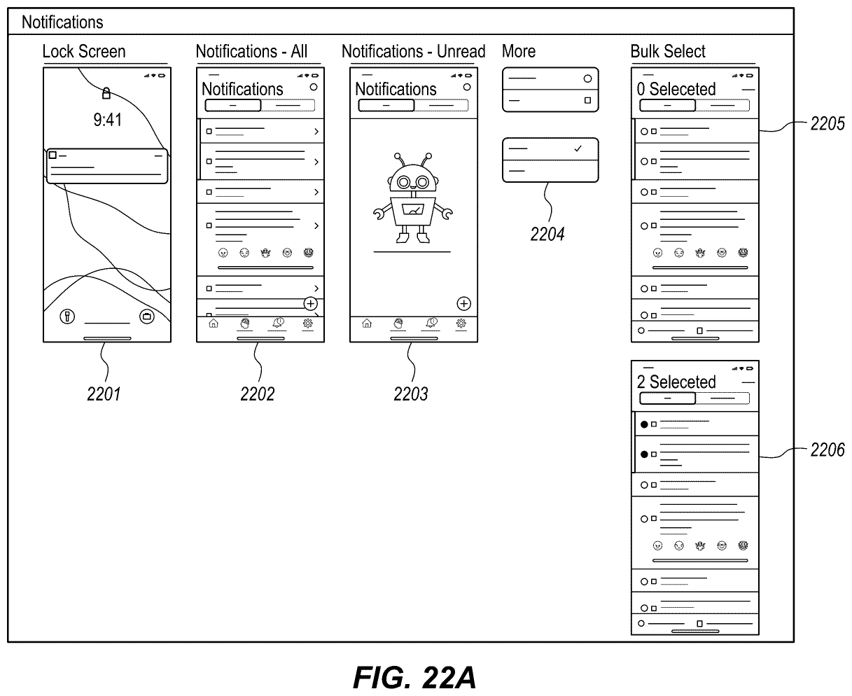

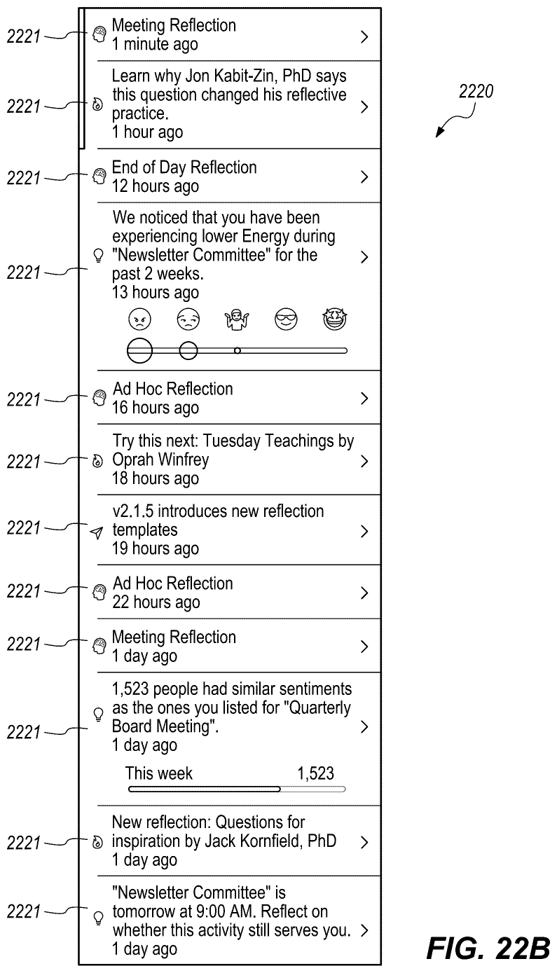

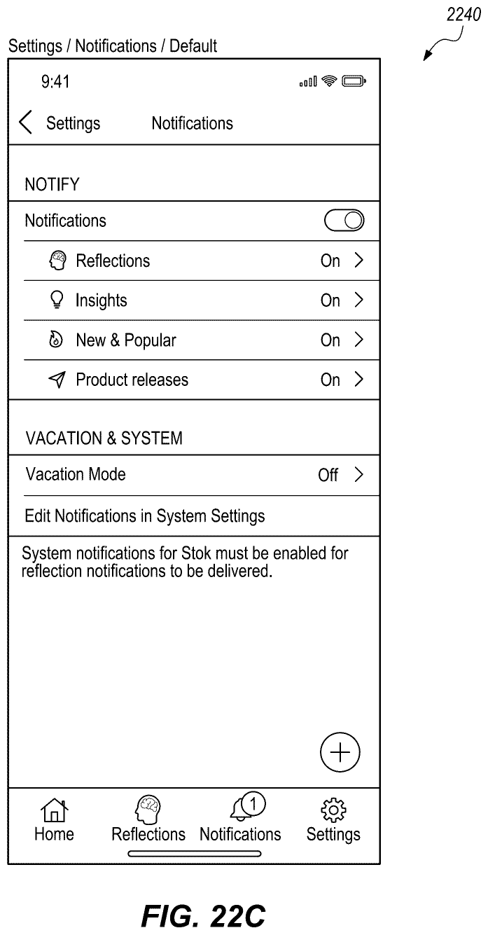

Referring now to FIGS. 22A through 22C, there are shown various screens that may be associated with Notifications Tab 1615, according to one embodiment.

As shown in FIG. 22A, a lock screen 2201 may be provided. For a reflection notification, the name of the reflection may be displayed in the title. Text in the subtitle may read, for example, “How was your [Name of Trigger] activity?”

As shown in FIG. 22A, screen 2202 may be provided, to show user 100 all of the notifications that they have received, in one place, so they can easily refer back to them later. This also makes it easy for user 100 to start or restart any reflections that they could not get to when they were initially notified.

Users 100 can easily filter the list to see All or only their Unread notifications. An example of Unread screen 2203 is shown in FIG. 22A. When there are no Unread notifications, a message may appear, including an optional drawing or animation saying, for example, “Fantastic! You're all caught up.”

More screen 2204 may be provided, to enable access to additional features. Bulk select screens 2205, 2206 may be provided, to allow selection of multiple items.

In at least one embodiment, users 100 can set the sort order of the notifications list to Newest or Oldest notifications first, and can bulk select to Mark as Read or Remove Selected notifications.

In at least one embodiment, several notification types may be provided, as follows:
Reflection: Prompt to start a reflection.
Insight: The system's observations that are made based on analysis of user's 100 reflections. These may also include observations made across users 100 of the app. If there's an associated metric behind the observation, a table or chart may be included with the data.
New & Popular: May include items, such as new items, Try This Next, Staff Picks, New Reflections, and/or the like.
Release: May include announcements of new features and version updates.

FIG. 22B depicts an example of a screen 2220 for accessing various reflections. User 100 can tap on any entry 2221 in screen 2220 to view the associated reflection.

In at least one embodiment, notification settings may be provided, including options to customize notifications for the different notification types. FIG. 22C depicts an example of a notification settings screen 2240 according to one embodiment.

### Reflections Tab 1613

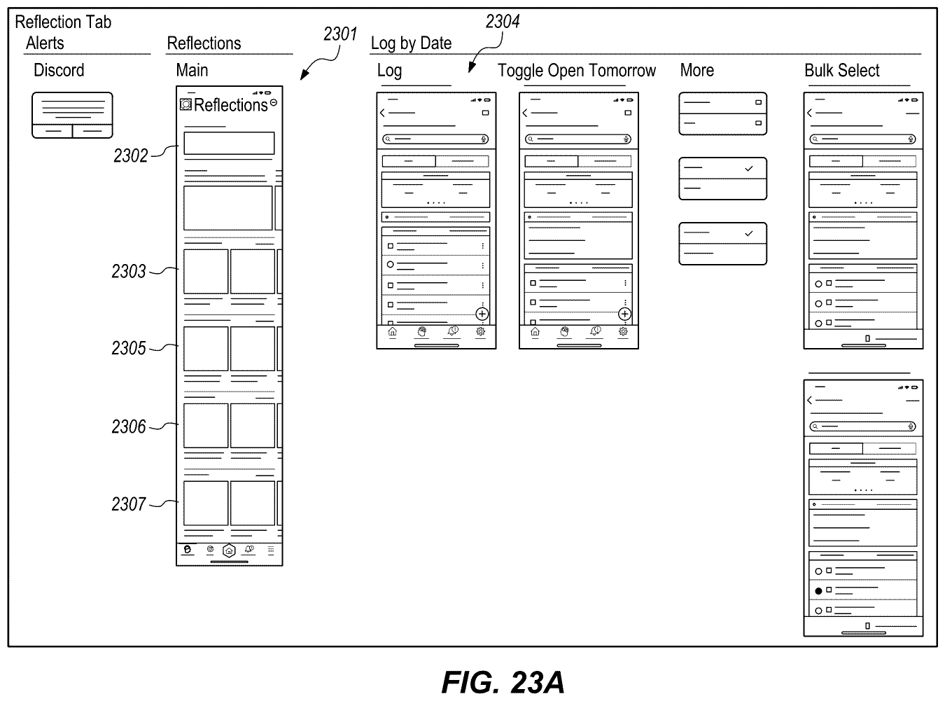

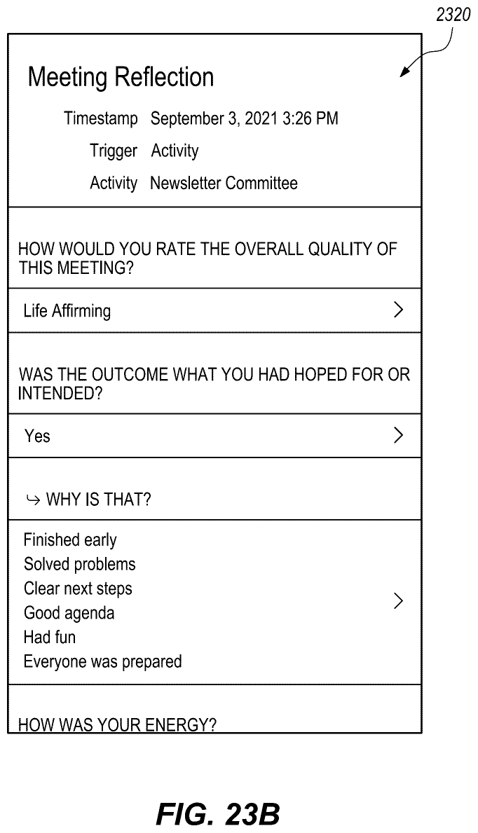

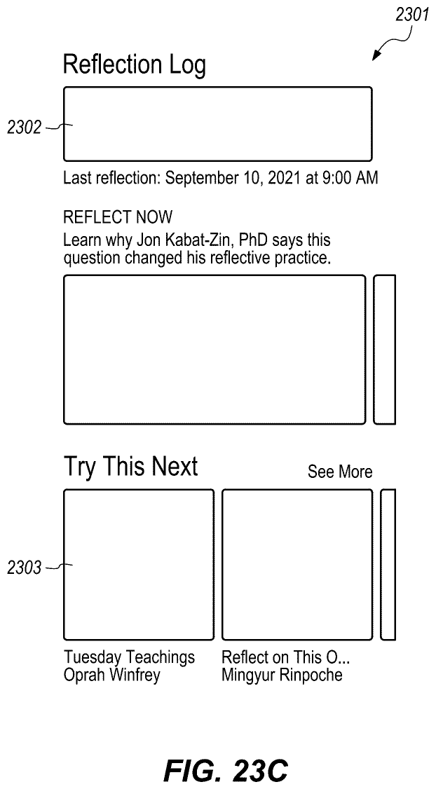

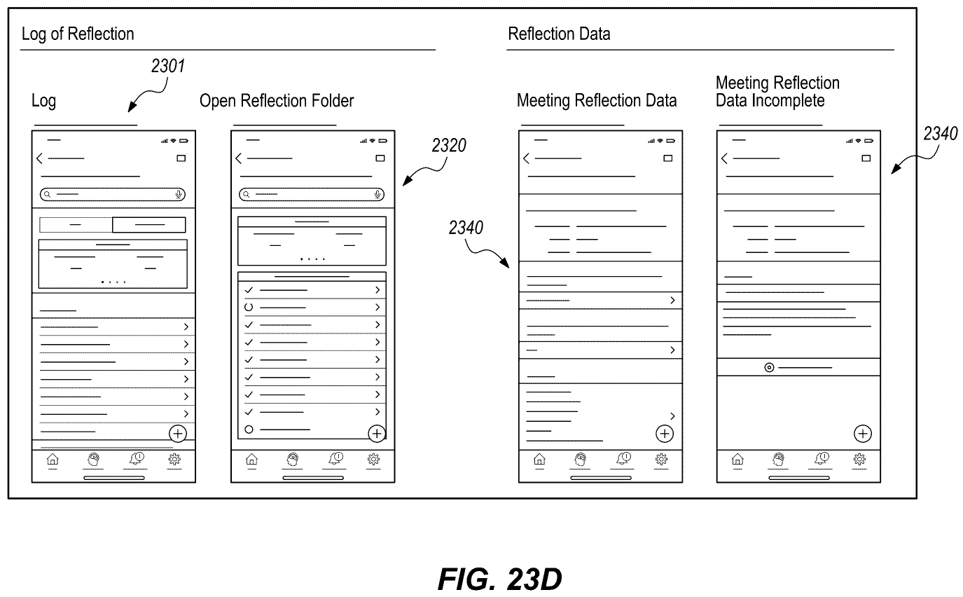

Referring now to FIGS. 23A through 23D, there are shown various screens that may be associated with Reflections Tab 1613, according to one embodiment.

In at least one embodiment, Reflections Tab 1613 may be used to display content and reflection practices by experts and celebrities in fields such as self-improvement, mindfulness, meditation, behavioral science, neuroscience, and/or the like.

As shown in the example of FIG. 23C (and in FIG. 23A), main screen 2301 may include the following elements:
Reflection Log 2302: A persistent area with a link to a Reflection Log 2304 that organizes all of user's 100 reflections. This may include a subtle reminder of their last reflection. Another section may be included as a place to announce new and popular content. User 100 may engage with the content and install one of the practices. In at least one embodiment, the top three new items may be shown.
Try This Next 2303: These may be hand-picked reflections for user 100 by their FDE 251.
New Reflections 2305: The rest of the new items may be shown here. After the top three items in the second section are pushed out by newer content, they may be shown here.
Staff Picks 2306: The favorite reflections from the team may be shown here.
Discover 2307: A mix of reflections that user 100 has not engaged with yet.
Tapping “See More” opens the respective section into a full screen modal with each item listed on its own line. User 100 can scroll horizontally through items in each section without having to click into it.

Log 2304 may provide the following features:
A segmented filter button collates the reflections by day or reflection name.
One or more key feedback metrics, allowing user 100 to scroll horizontally through these to get a sense for how they are doing. The goal is to show user 100 that reflecting allows the system to help them achieve their desired goals/actions.
   Time spans: This week, last week, this month, last month.
   Values: Number of reflections completed, number of actions taken as a result.
Completed reflections may be displayed with a checkmark, while incomplete ones may be displayed with a dashed circle. In at least one embodiment, completed reflections may be edited in the Data view.

Incomplete reflections can be started from the Data view

In at least one embodiment, reflections may be collated by day, as follows:
Tomorrow: grayed out and collapsed because the reflections have not been triggered for user 100 yet. This is a preview allowing user 100 to plan their day and see what is on deck.
Today, Yesterday, [Day]: shows all of the reflections that have been triggered for the day.
Any reflections that are yet to be triggered are displayed like those in Tomorrow.
List can be sorted by Newest (default) or Oldest

In at least one embodiment, reflections may be collated by reflection name, as follows:
A list of reflection names may be displayed for user 100. When tapping on a reflection name, user 100 can see all of the times they have reflected, then select a specific instance to see the data.
Collations may be performed by timestamp or question (where users 100 can see a summary of all answers in dashboard form).
List can be sorted by name Ascending (default) or Descending.

In at least one embodiment, reflection data may be shown as follows:
Header: Name of the reflection
Timestamp: Month d, YYYY h:m AM/PM
Trigger:
   [Dynamic Trigger] (Interval, Schedule, Activity, Location): [List the summary of the setting]
   Display the question and the answer.
   Clicking on the answer opens the Edit Data screen where user 100 can edit their answer.

FIG. 23B depicts an example of a meeting reflection screen 2320, according to one embodiment.

FIG. 23D depicts examples of log screen 2301, a meeting reflection screen 2320, and screens 2340 for displaying reflection data, according to one embodiment.

### Custom Reflections

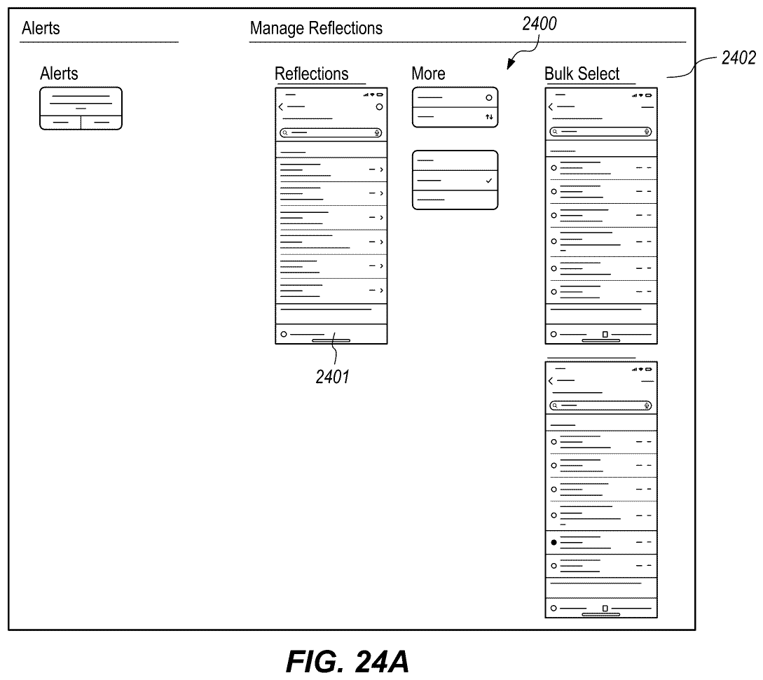

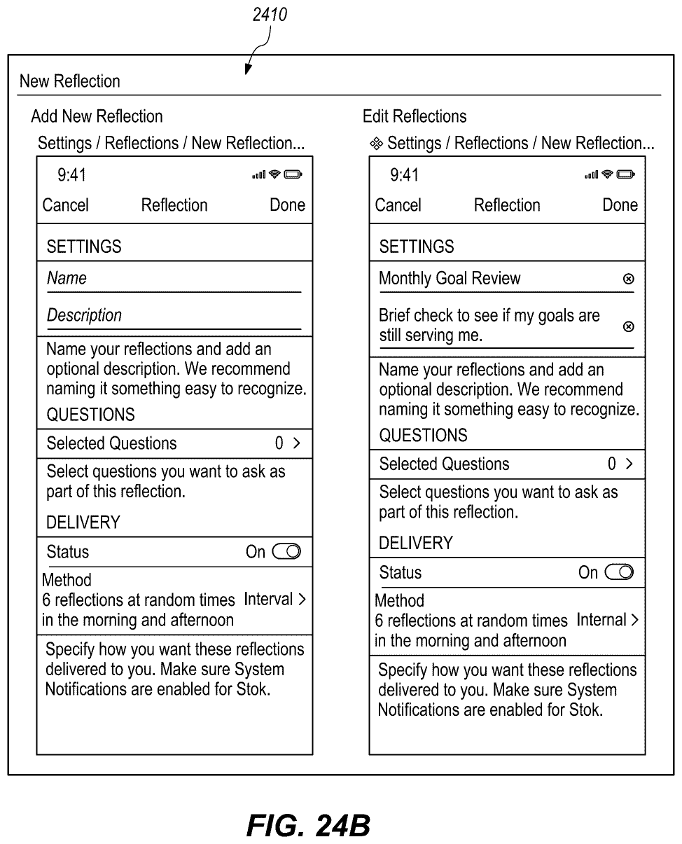

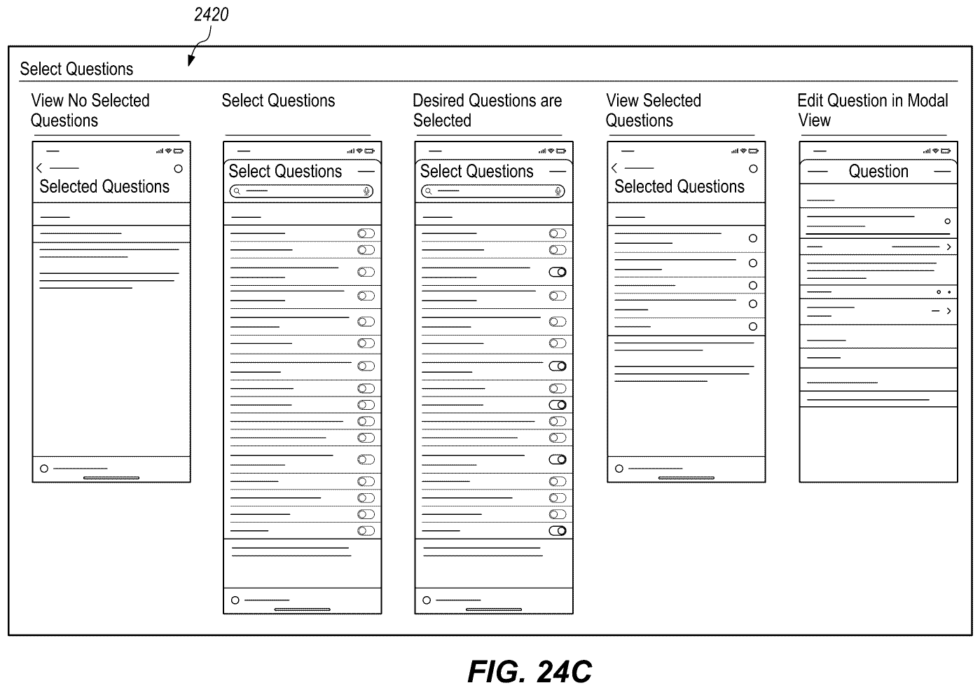

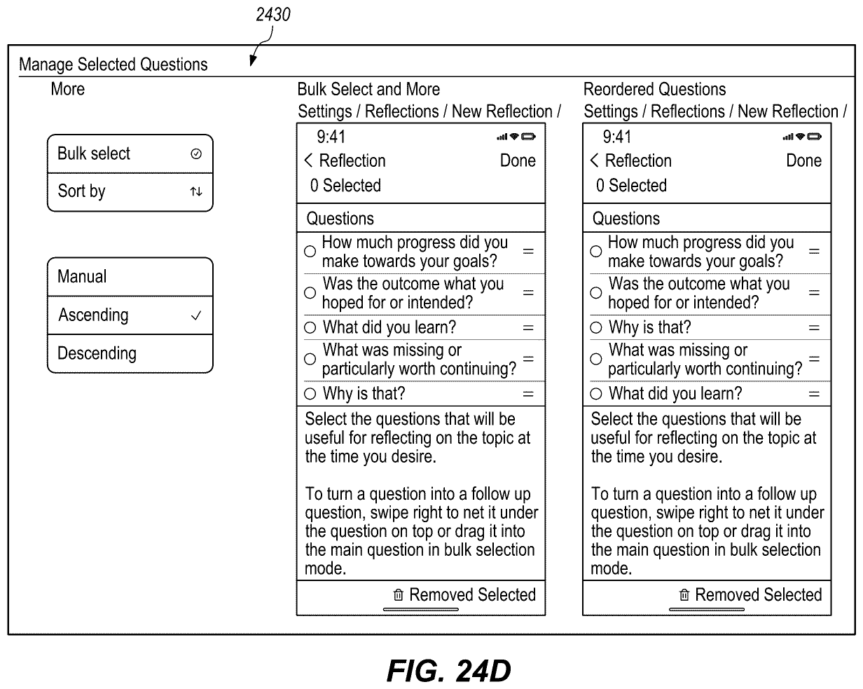

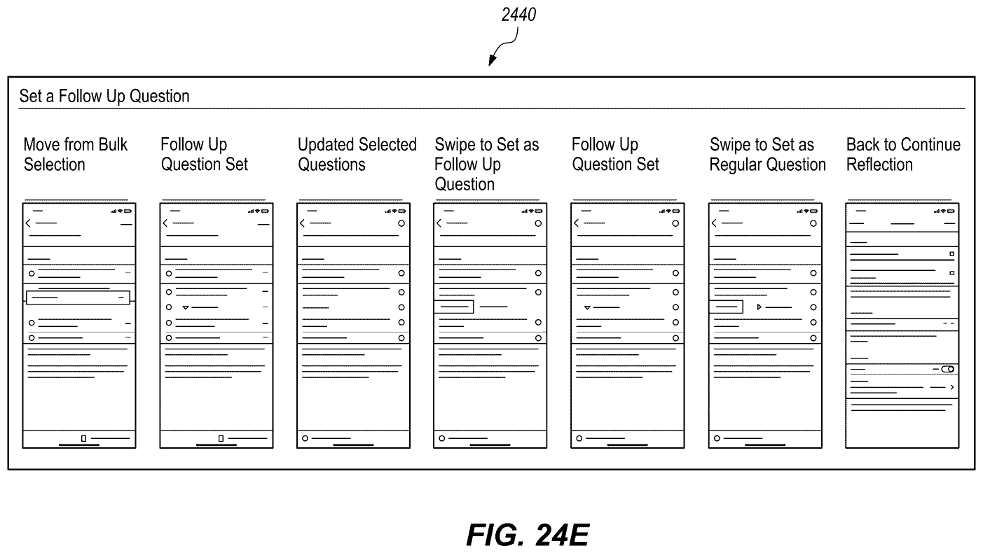

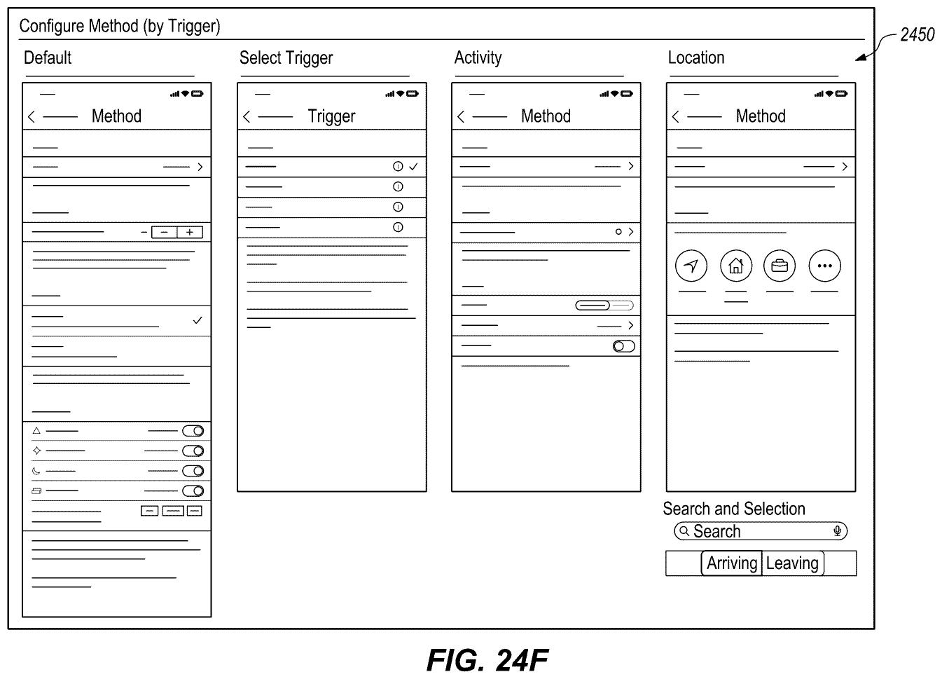

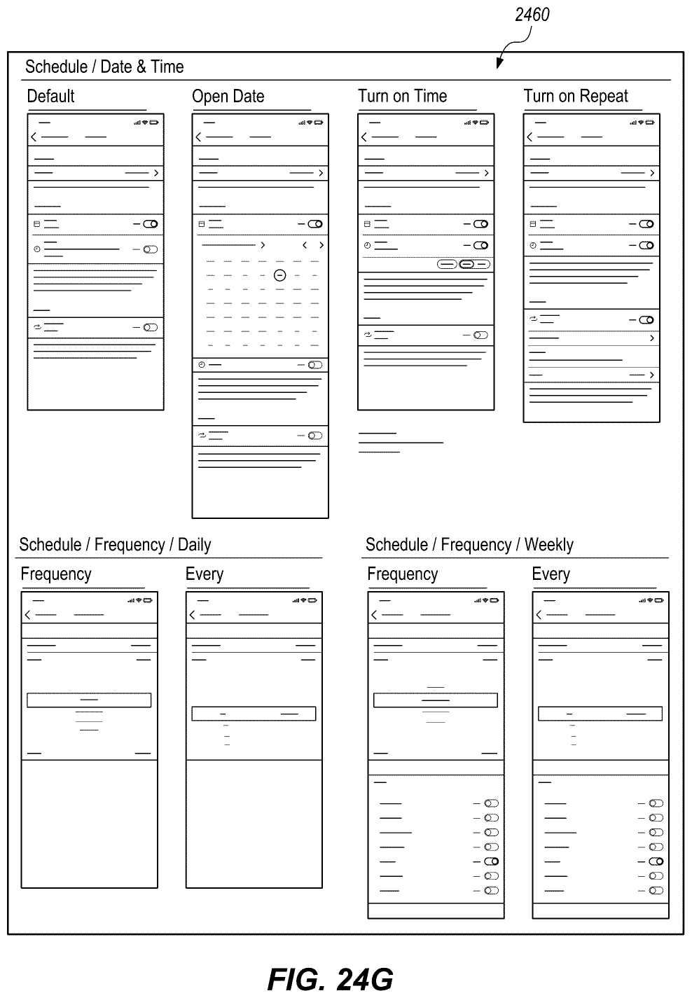

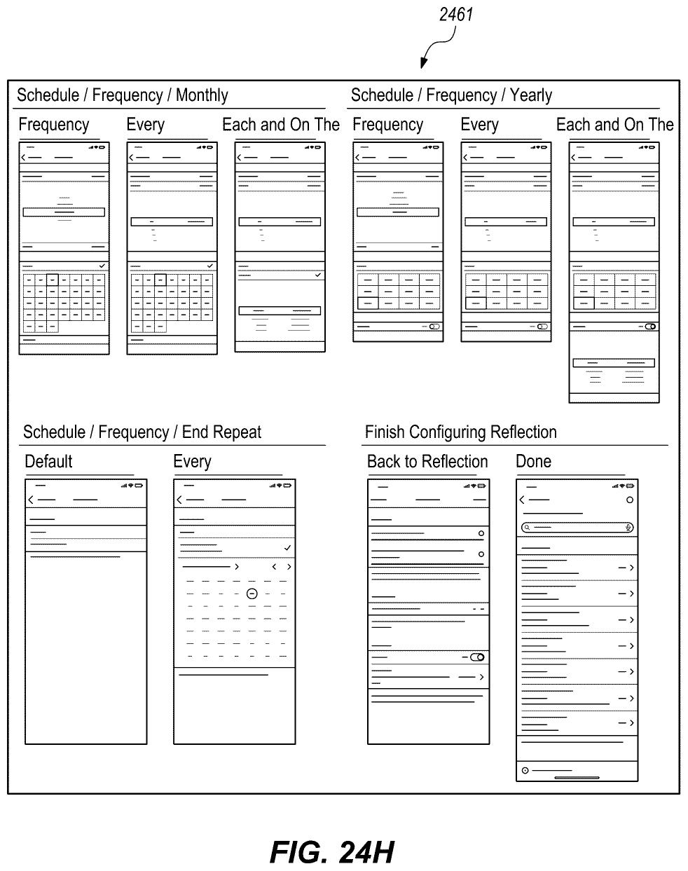

Referring now to FIGS. 24A through 24H, there are shown various screens that may be associated with functionality for creating custom reflections according to one embodiment.

The following functionality may be provided:
Manage Reflections
   List of reflections
   Bulk select to remove or turn on/off (delivery).
   Sort: manual, ascending, descending
New Reflection
   Name and description
   Questions
   Delivery:
      Status on/off
      Delivery options
Configure Delivery
   Delivery Method:
      Random: Time(s) of Day
      Scheduled:
         Repeats
         Repeats every
         Time
         Starts on
         Ends on
      Activity Dependent
         From calendar
         Deliver before/after
         Batching
         Time span

Each of the above will be described in turn.

### Manage Reflections

User 100 can manage items. Example screens 2400 are shown in FIG. 24A. The options may be available:
Add a New Reflection 2401
   Sort
   Manual
   Ascending (default)
   Descending
Bulk Select 2402
   Turn On/Off
   Remove Selected

### New Reflection

An example screen 2410 is shown in FIG. 24B. When creating a new reflection, user 100 can configure:
Settings
   Name
   Description
Selected Questions
Delivery
   Status
   Method

### Select Questions

Example screens 2420 are shown in FIG. 24C. When user 100 selects questions for a reflection, they have the following options:
Pick from the existing reusable questions they have created in the Questions section; or
Create a New Question (in modal overlay) that will both be added to the Questions section and selected for the reflection.

Toggling on a question selects it for the reflection. Pressing “Done” applies the toggle selections and adds the selected questions to the new list of Selected Questions. If user 100 wants to edit the main question they selected, they can click on the info icon. There is no limit to the number of selected questions.

### Manage Selected Questions

Users 100 can manage their list of questions. Example screens 2430 are shown in FIG. 24D. The options may be available:
Sort
   Manual
   Ascending (default)
   Descending
Bulk Select
   Remove Selected
      Set a Followup Question

Example screens 2440 are shown in FIG. 24E. In at least one embodiment, user 100 may have two (or more) methods for setting a question as a followup question to a main question:
In bulk select mode: Dragging the question into another question to nest it within as a followup or dragging it out to undo; or
In default mode: Swiping the question right to expose options to convert the question to a “Follow Up” or “Regular” question.

Any question can be a followup question.

There is no limit to the number of followup questions within one question. In at least one embodiment, there can only be one level of followup questions (i.e., a followup question cannot be nested within another followup question).

### Configure Method

In at least one embodiment, user 100 is able to configure how they want their reflection delivered to them.

A trigger is the type of event that prompts the reflection. Example configuration screens 2450 are shown in FIG. 24F. In at least one embodiment, the following parameters may be configured:
Interval (default)
   Frequency—number of reflections per day (default is 6)
   Interval
      Random (default)—stochastically timed
      Regular—evenly distributed between the start of Morning and Bedtime
   Time of day
      User 100 can determine the time frames within which when a reflection can be delivered.
      If Bedtime is selected, user 100 has the option to set a cushion for deliver before bedtime so they have time to complete their reflection before going to sleep.
Schedule
Activity
   Pick an activity from calendar
   Timing—before or after an activity by a certain time span
Location
   Pick a location from current location, favorites, or create a custom location input similar to the location-based question.

### Schedule

Example configuration screens 2460, 2461 are shown in FIGS. 24G and 24H. The following parameters may be configured:
Date—Turned on by default and Today is selected by default.
Time—Turned off by default. When turned on, the next full hour is the default.
Repeat
   Repeat—Turned off by default. When turned on these options are visible:
      Frequency
      Starts (static info based on Date)
      Ends
   Frequency Options
      Daily
      Weekly
      Monthly
      Yearly
   Every Options
      Value+Daily, Weekly Monthly, Yearly
   Each Options
      Day(s) of the Week (starting with Monday)
      Day(s) of the Month
      Month(s)
   On the Options
      Nth+Day of the Week (starting with Monday)
   End Repeat
      Never (default)
      End Repeat On-Default is today

Once user 100 is done with adding a New Reflection, it may be saved to the list.

### Custom Survey Questions

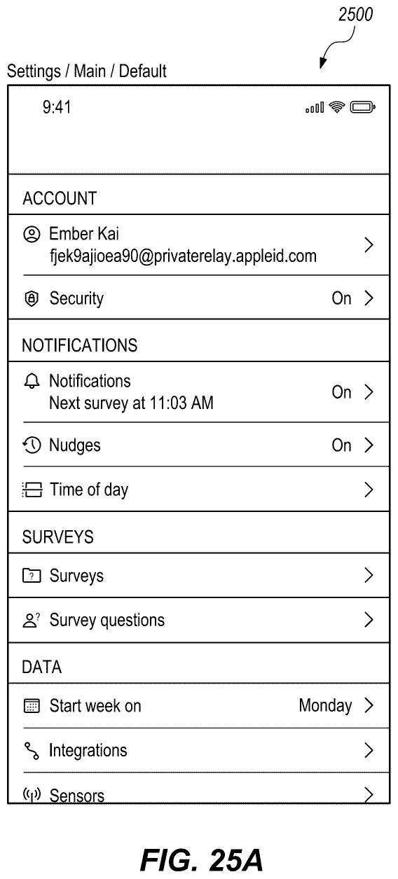

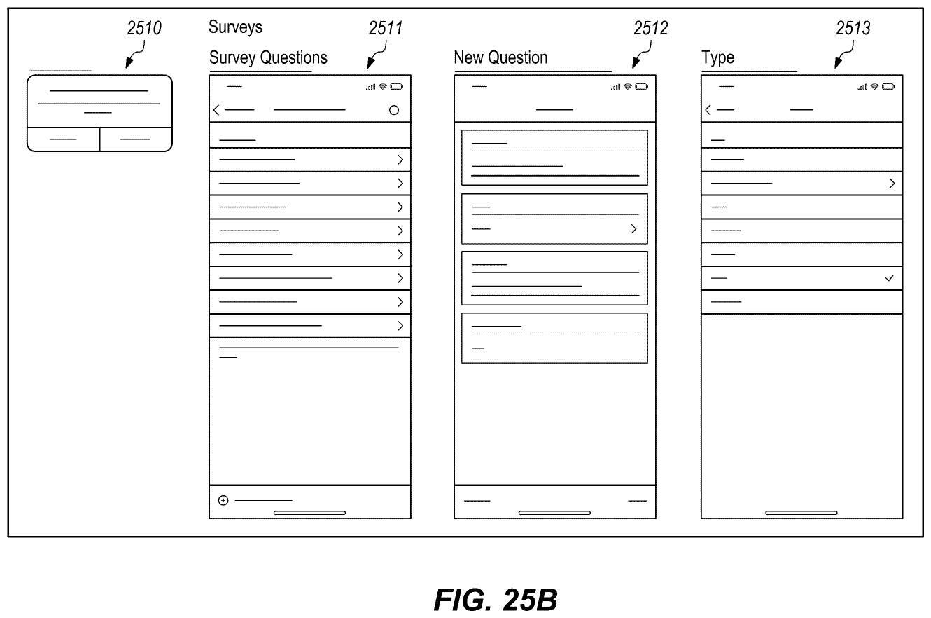

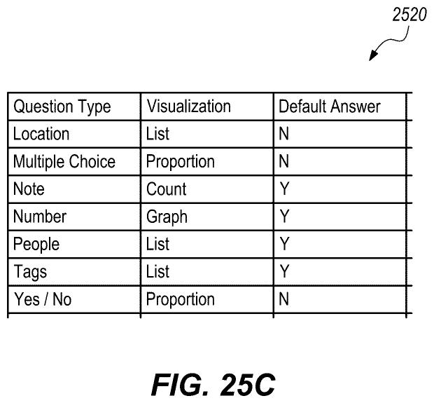

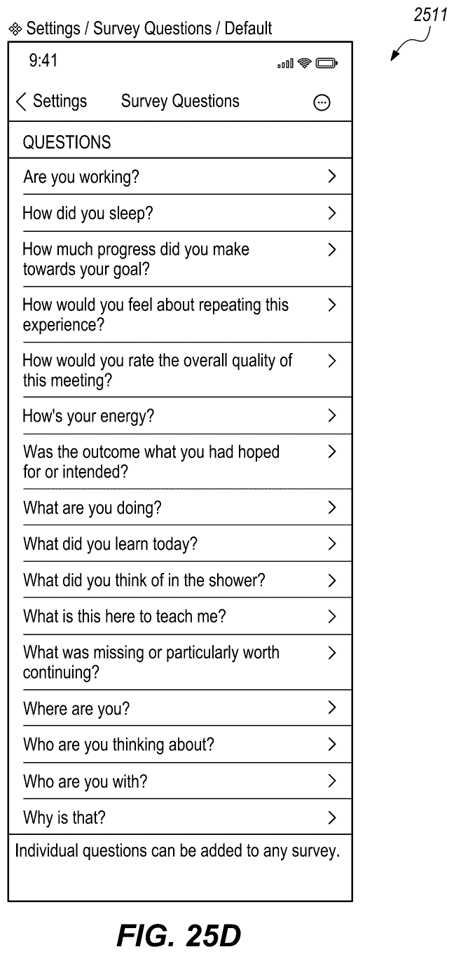

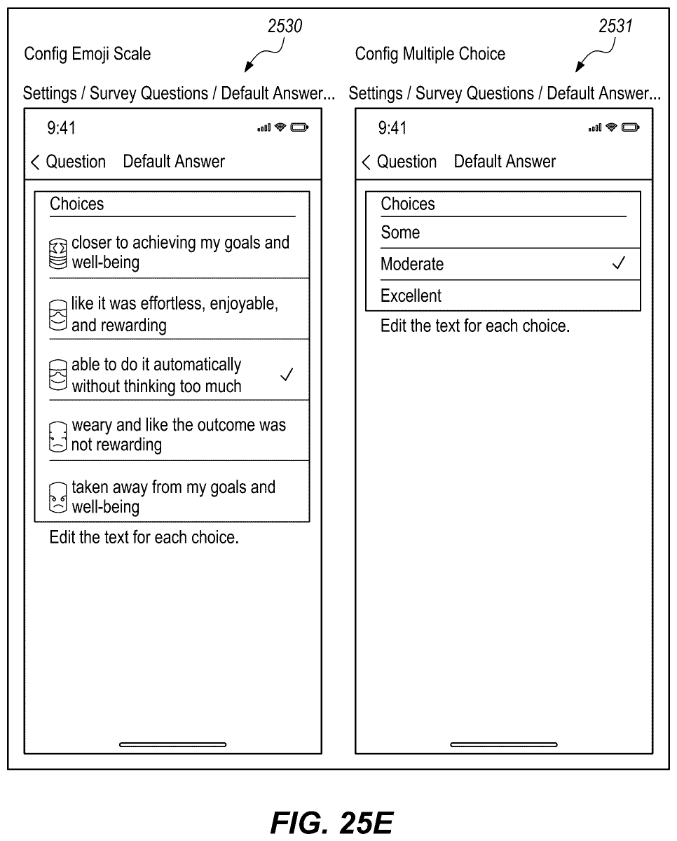

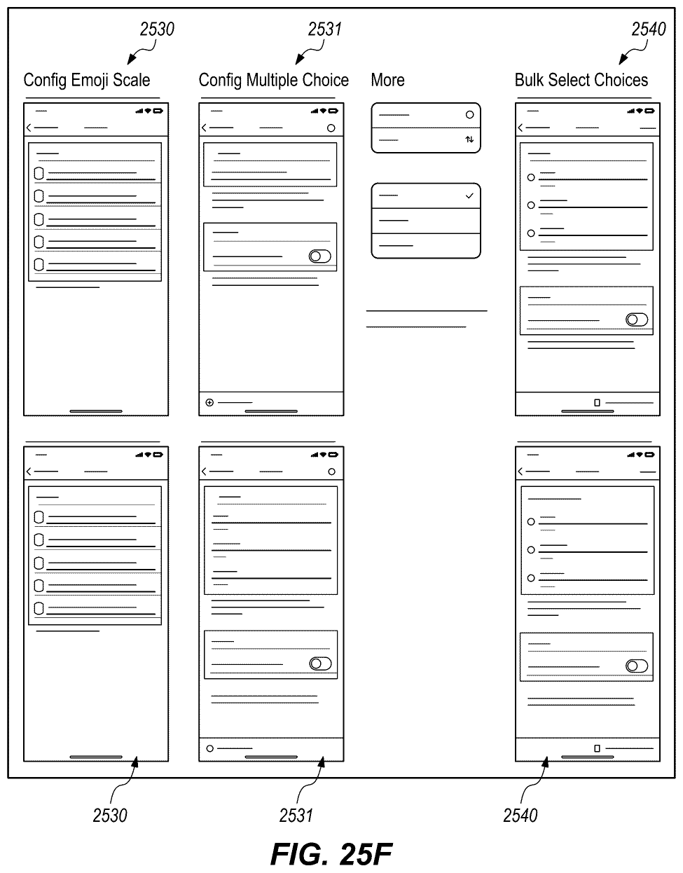

In at least one embodiment, users 100 can customize questions that can be reused in different surveys. In addition, users 100 can edit/delete existing questions and/or add new ones as desired. FIGS. 25A through 25F depict various screens that may be associated with such functionality according to one embodiment.

Functionality may be provided for adding a new question. User 100 can provide the following information:
Question: user 100 can enter the question they want to ask
Type: user 100 can select the type of question they want to ask (default: tags)
Default Answer: user 100 can define the default answer to start with, which may depend on the question type
Visualization: this may be static depending on the question type, or can have different options for some question types

In Reporter, there are Time of Day controls. In at least one embodiment, user 100 can set the Time of Day on the survey level. This gives user 100 greater control over when specific questions are delivered to them.

Type may include any of the following, for example:
Location
Multiple choice
Note
Number
People
Tags
Yes/No

User 100 may bulk select survey questions, and can reorder and/or delete questions. If appropriate, questions may be grouped with one another.

In at least one embodiment, user 100 can configure choices, for example for emoji scale and/or multiple choice questions. This may include, for emoji scale questions, changing text and/or choosing emojis. For multiple choice questions, user 100 may have the option to swipe to reveal a delete button and/or bulk select/reorder.

In at least one embodiment, user 100 can configure a default answer by selecting from a list of emojis or multiple choices.

FIG. 25A depicts an example of a settings screen 2500.

FIG. 25B depicts examples of screen 2510 for prompting the user to discard a draft of a question, screen 2511 for reviewing and specifying survey questions, screen 2512 for providing a new question, and screen 2513 for specifying an expected response type. FIG. 25D depicts an example of screen 2511 in more detail.

FIG. 25C depicts a table 2520 for specifying a question type, visualization, and default answer.

FIG. 25E depicts examples of screen 2530 for configuring an emoji response scale and screen 2531 for configuring a multiple choice response scale.

FIG. 25F depicts examples of screens 2540 for specifying bulk select choices.

### Notifications and Integrations

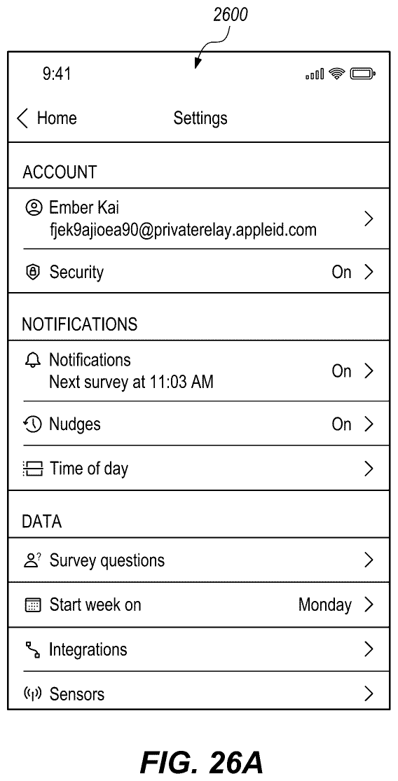

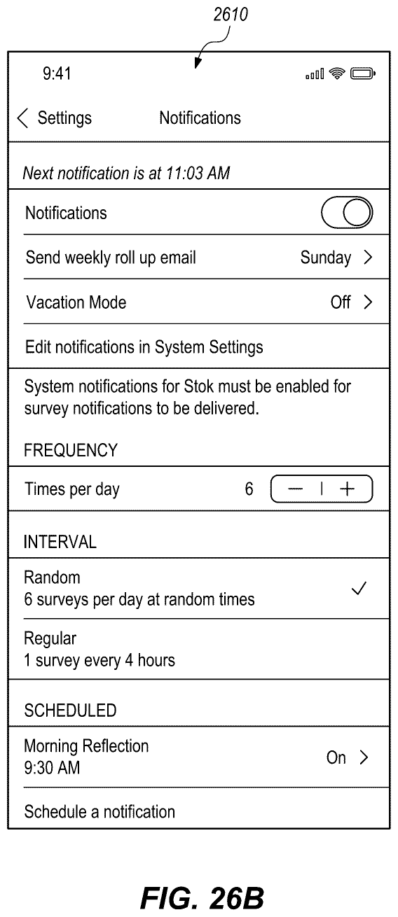

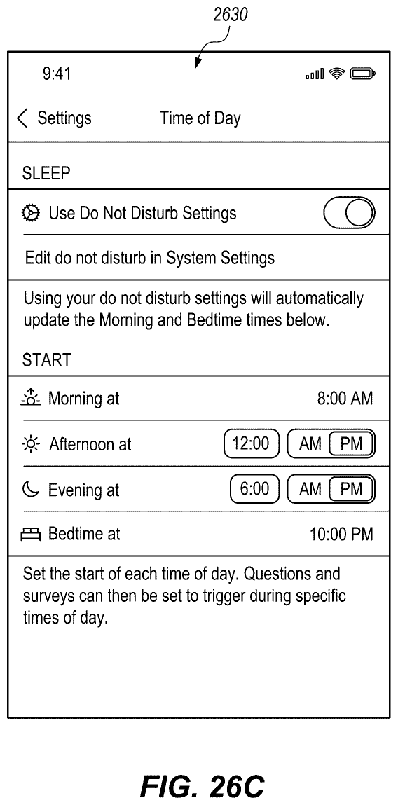

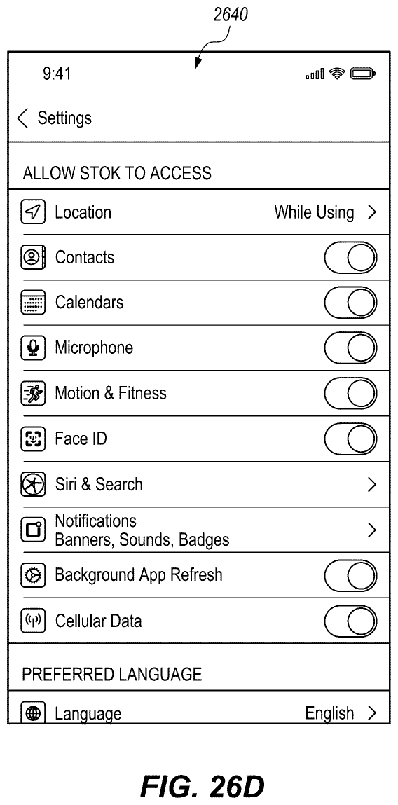

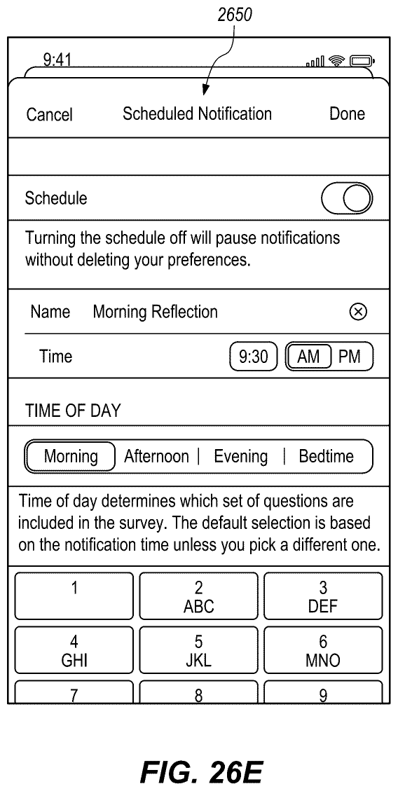

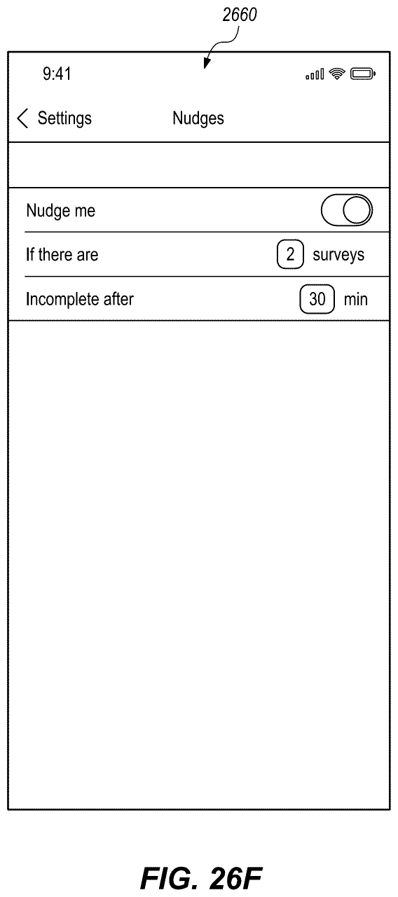

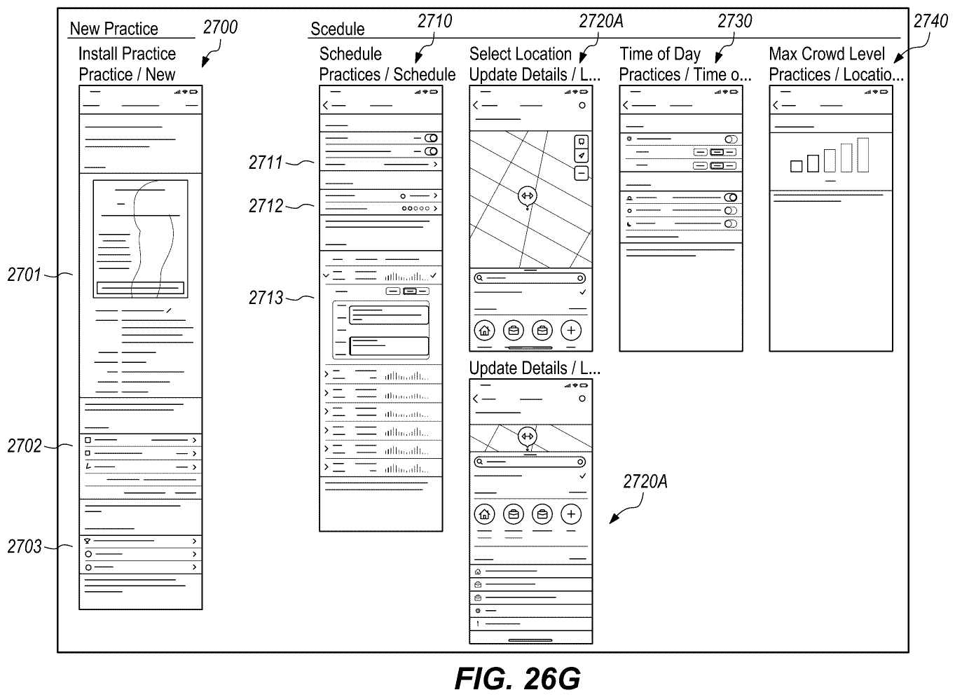

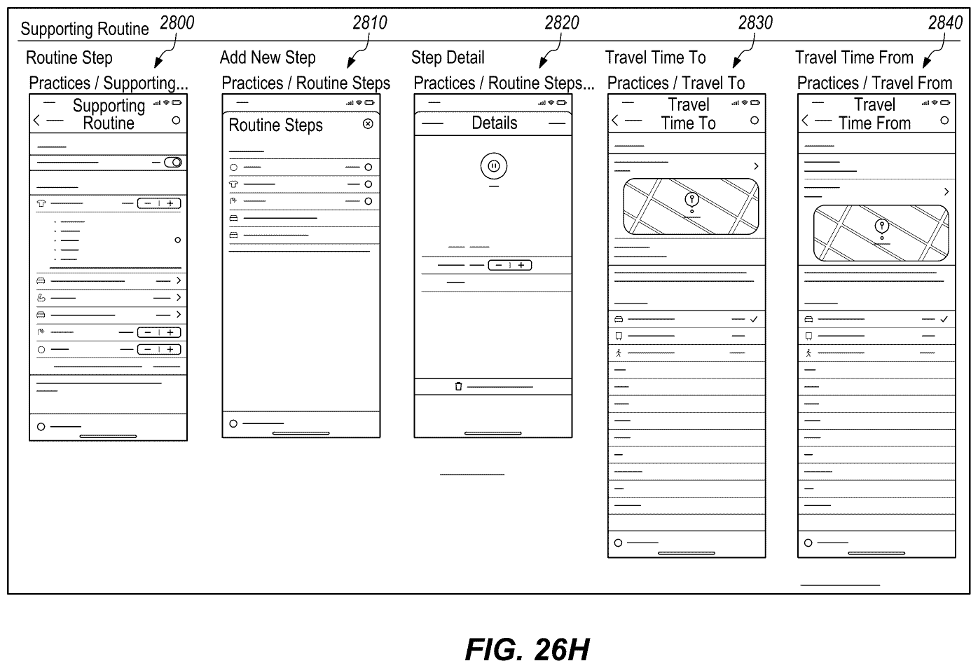

Referring now to FIGS. 26A through 26F, there are shown depict various screens that may be associated with functionality for configuring notifications and integrations according to one embodiment.

The following elements may be configured via the Notifications section:
Notifications
Nudges
Time of Day:

Time of Day specifies when morning, afternoon, evening, and bedtime starts, with the option to use Do Not Disturb settings to set morning and bedtime automatically. Time of Day categories can also be used to specify the types of questions triggered in a survey.

FIG. 26A depicts an example of a settings screen 2600.

FIG. 26B depicts an example of a screen 2610 for configuring notifications.

FIG. 26C depicts an example of a screen 2630 for configuring time of day settings.

FIG. 26D depicts an example of a screen 2640 for configuring access to other functions of the user's device.

FIG. 26E depicts an example of a screen 2650 for configuring a scheduled notification.

FIG. 26F depicts an example of a screen 2660 for configuring nudges.

### Meeting Reflections

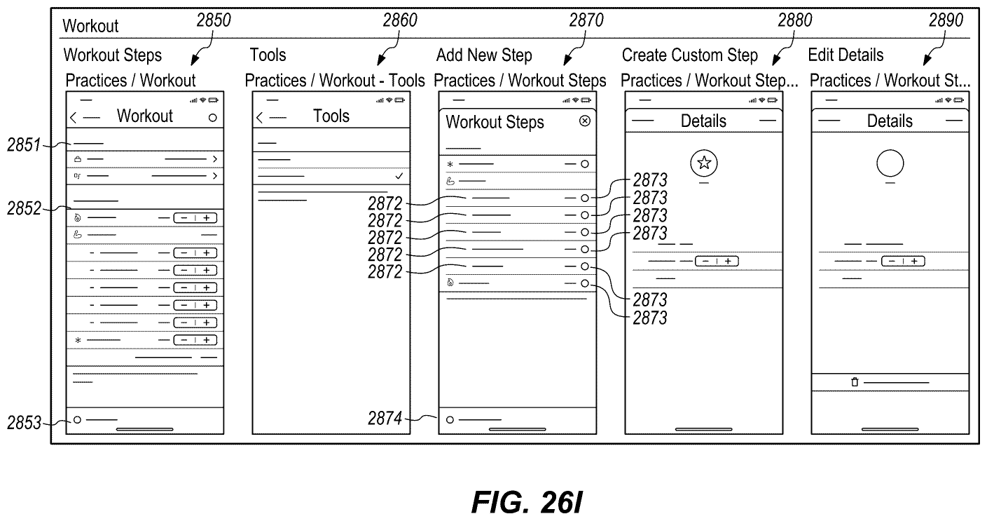

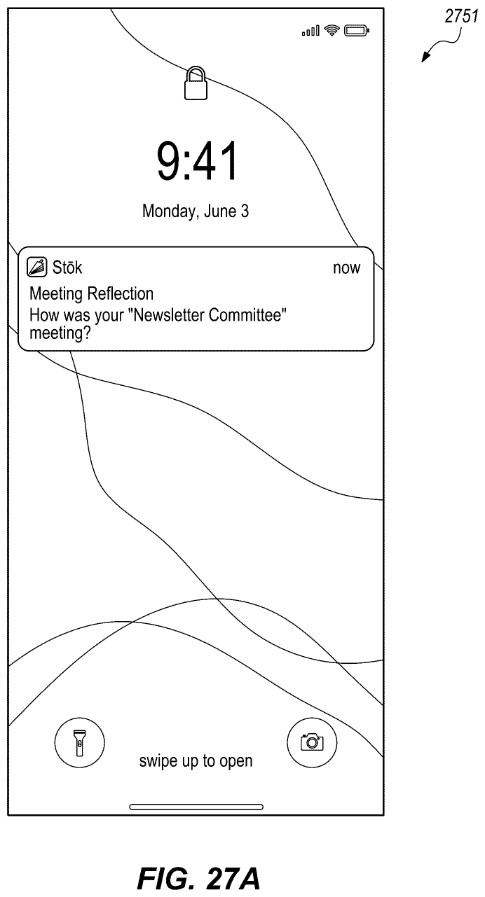

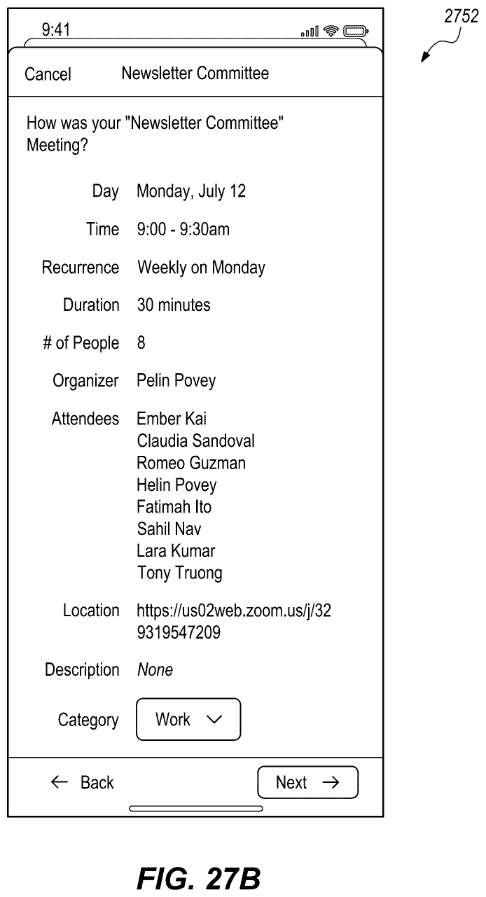

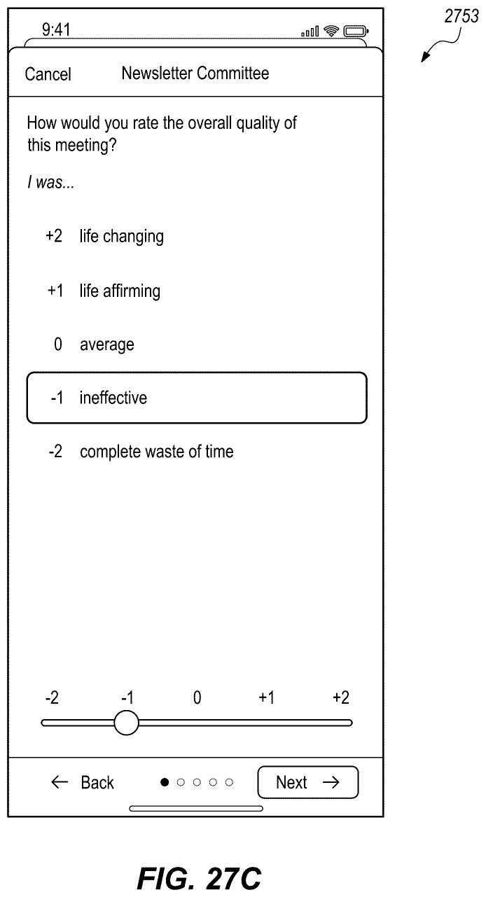

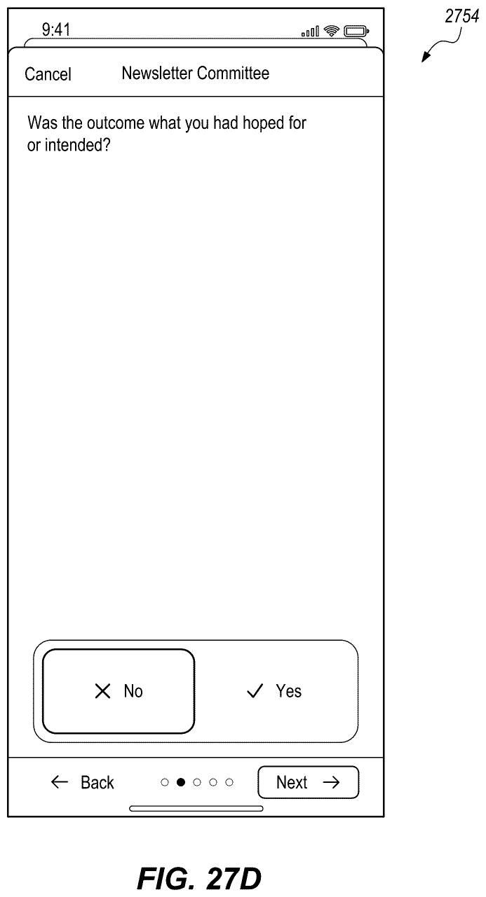

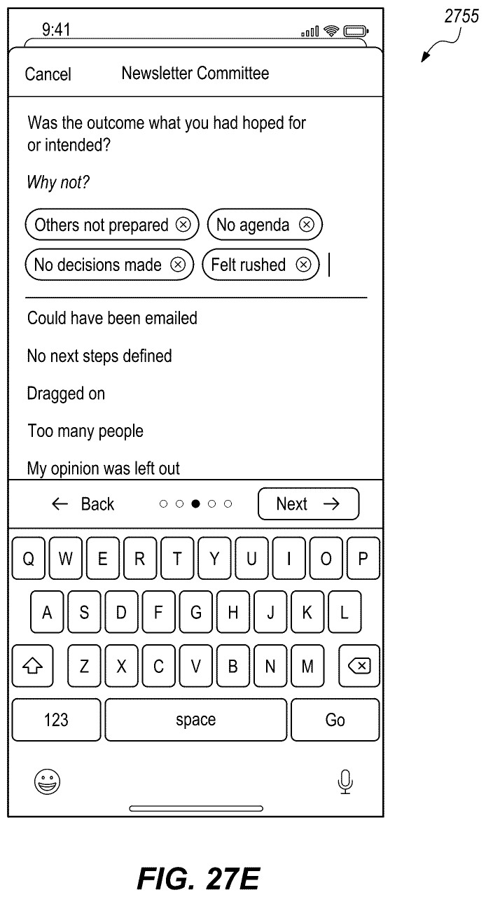

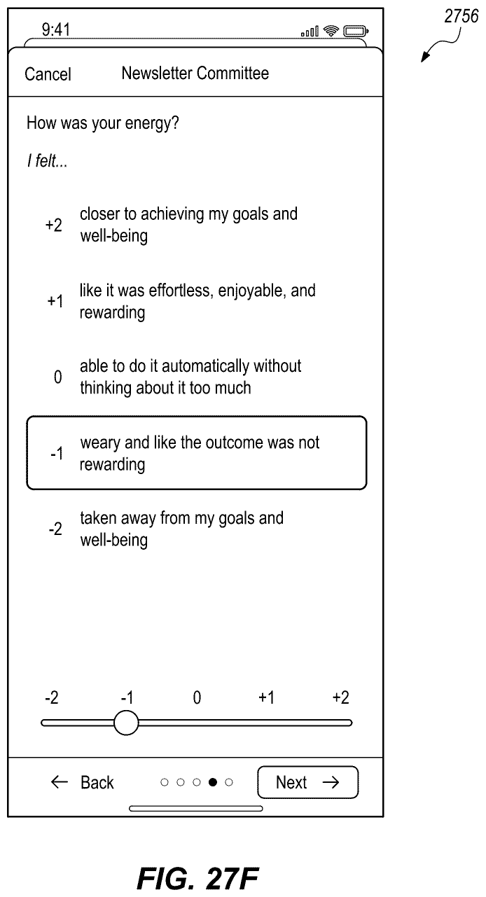

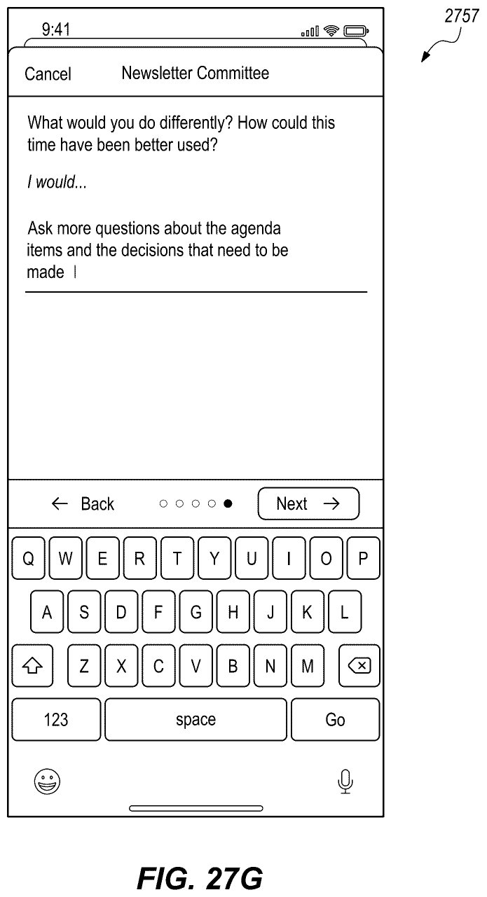

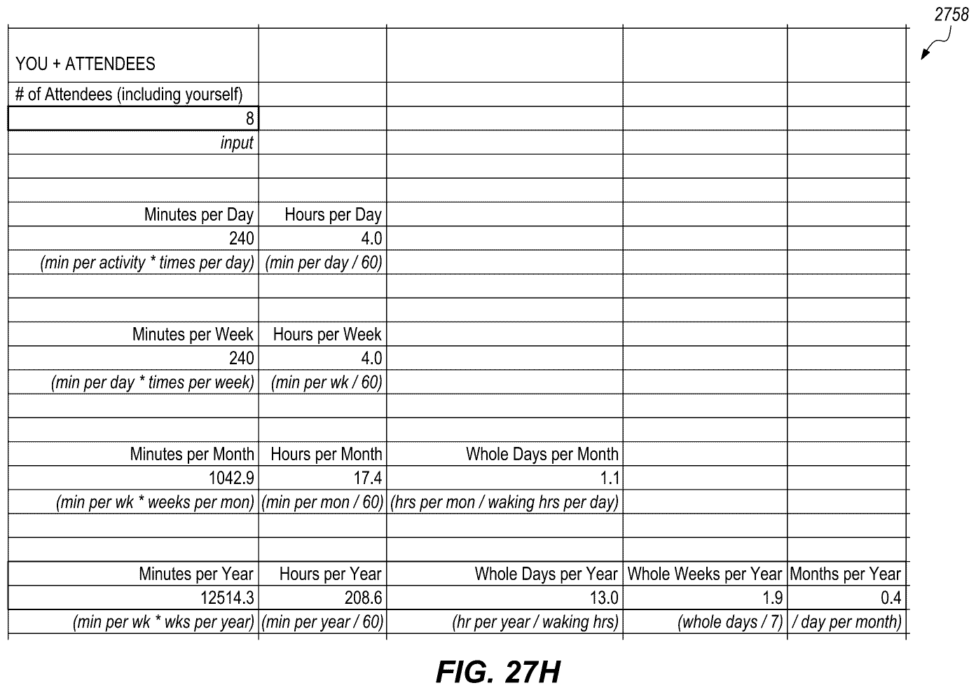

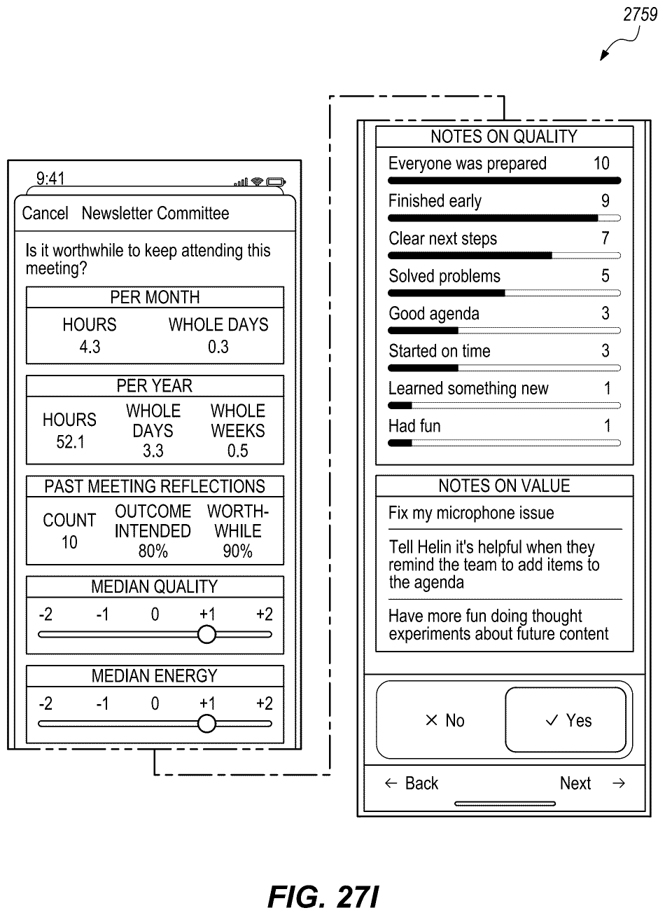

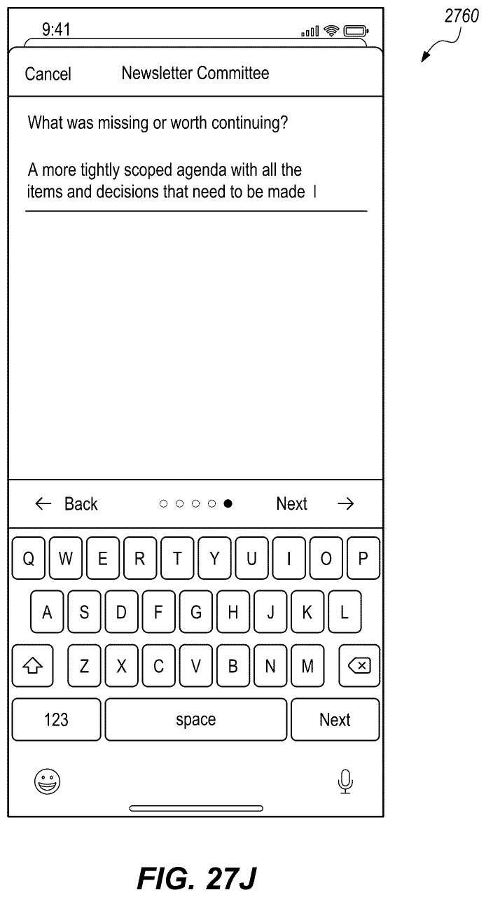

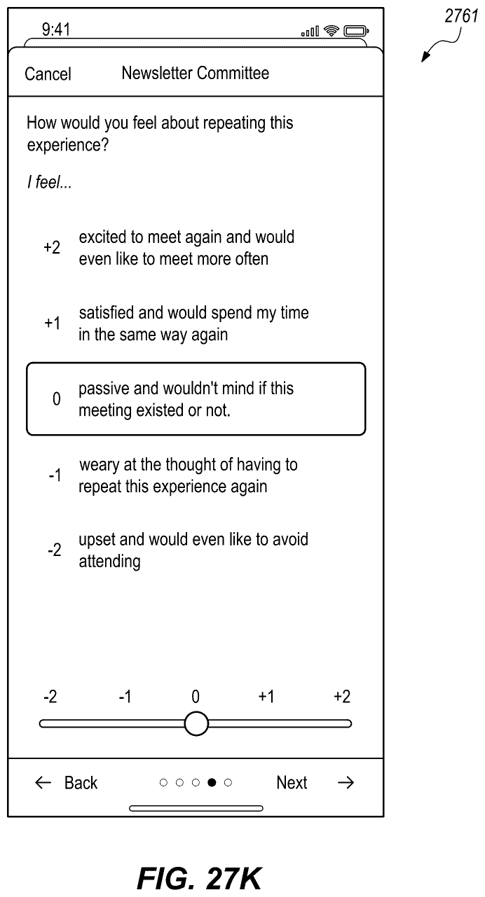

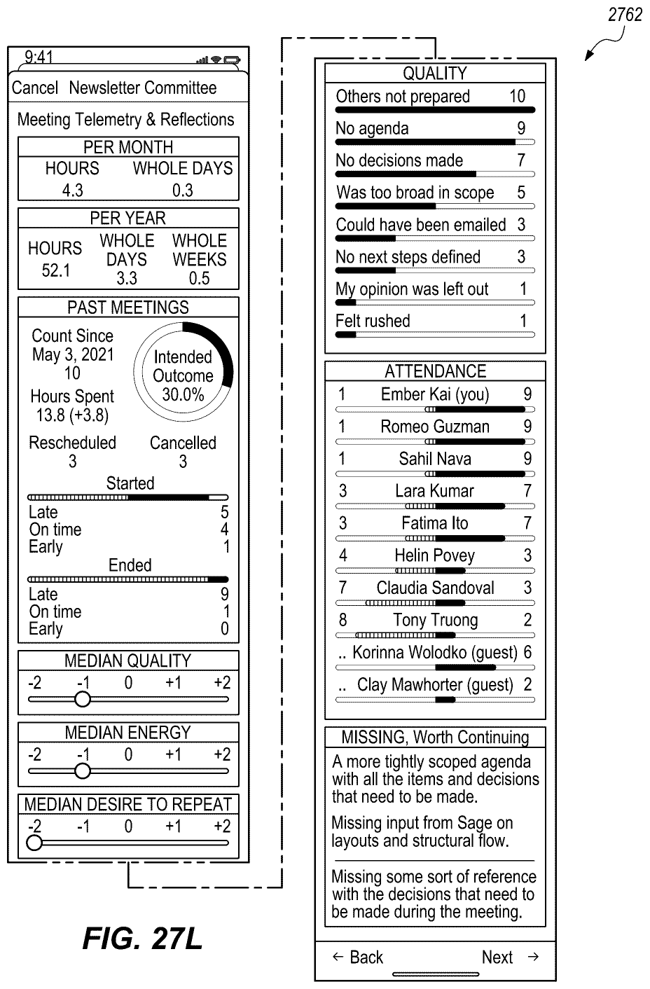

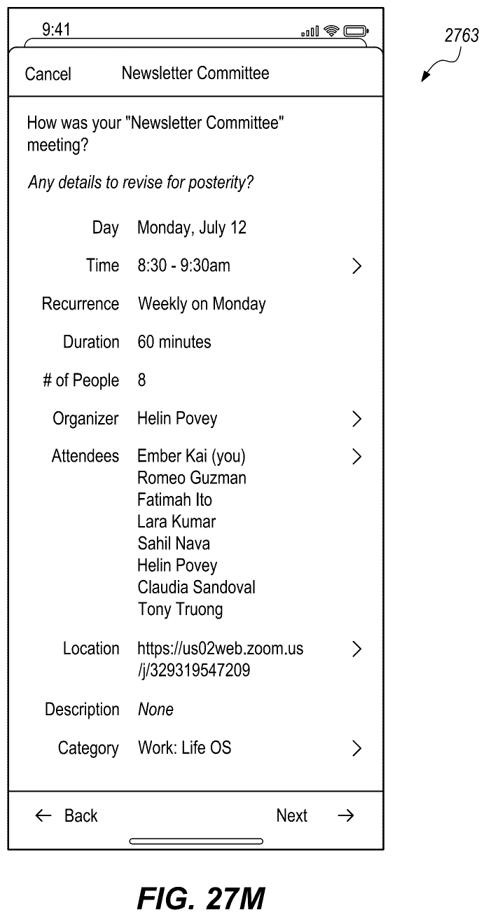

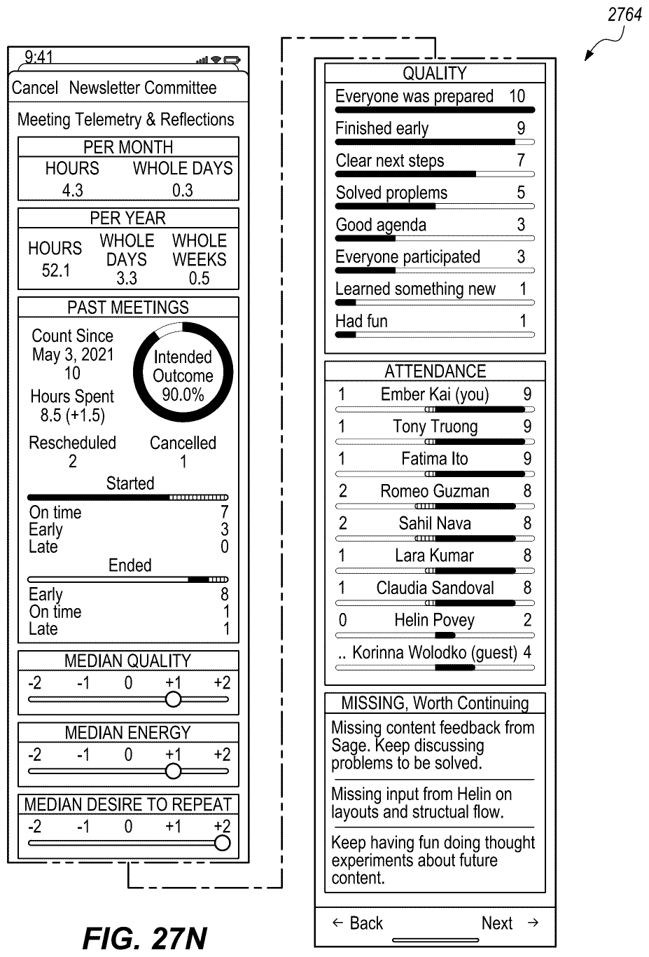

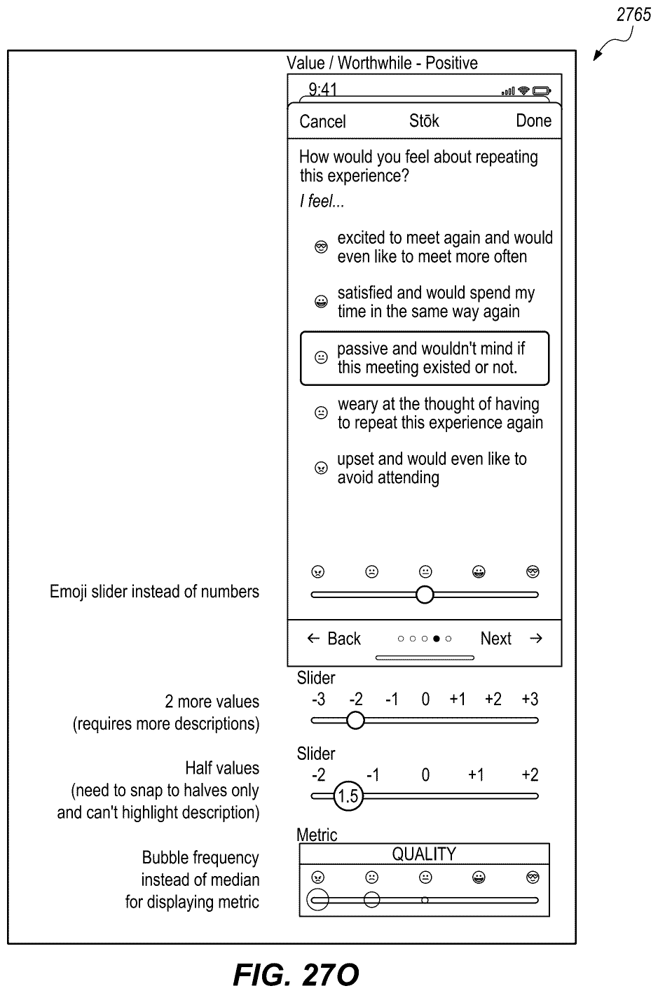

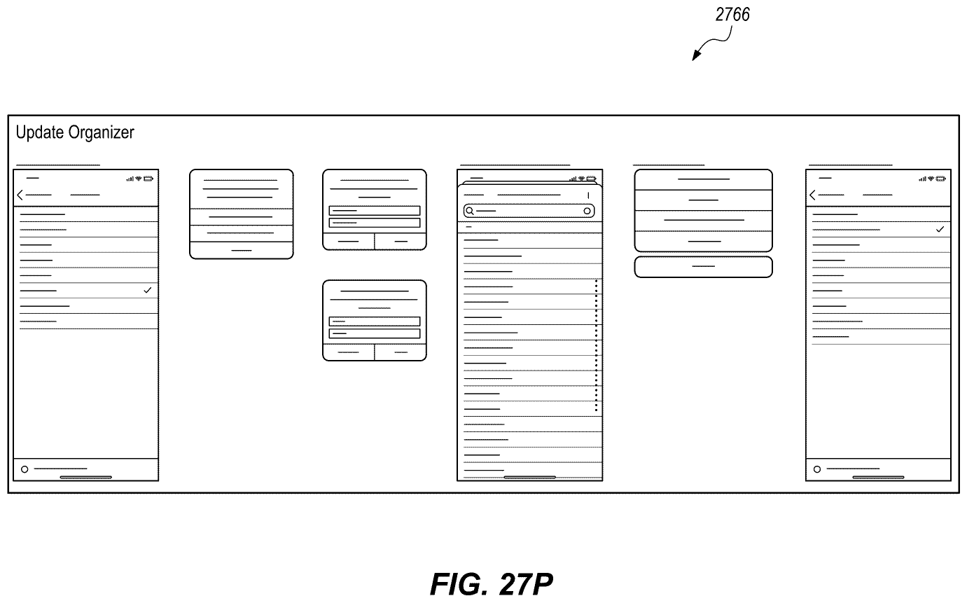

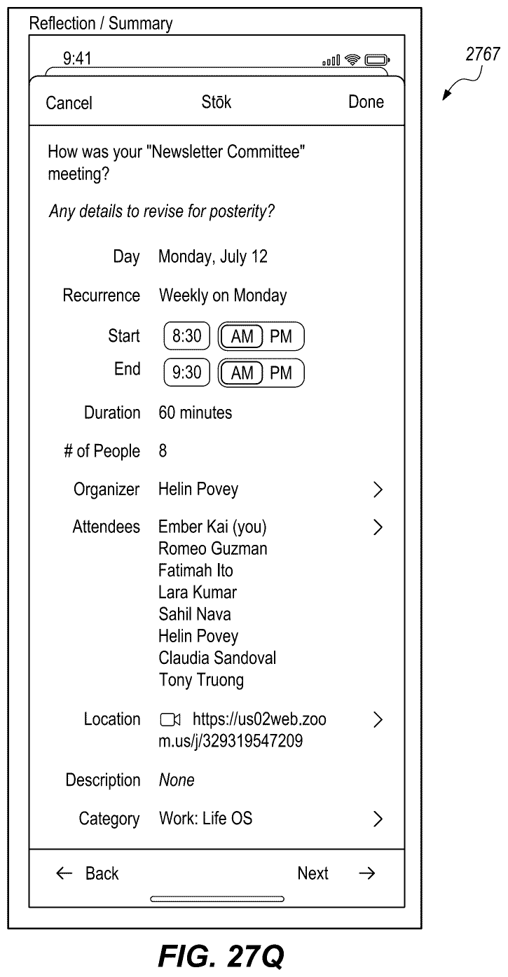

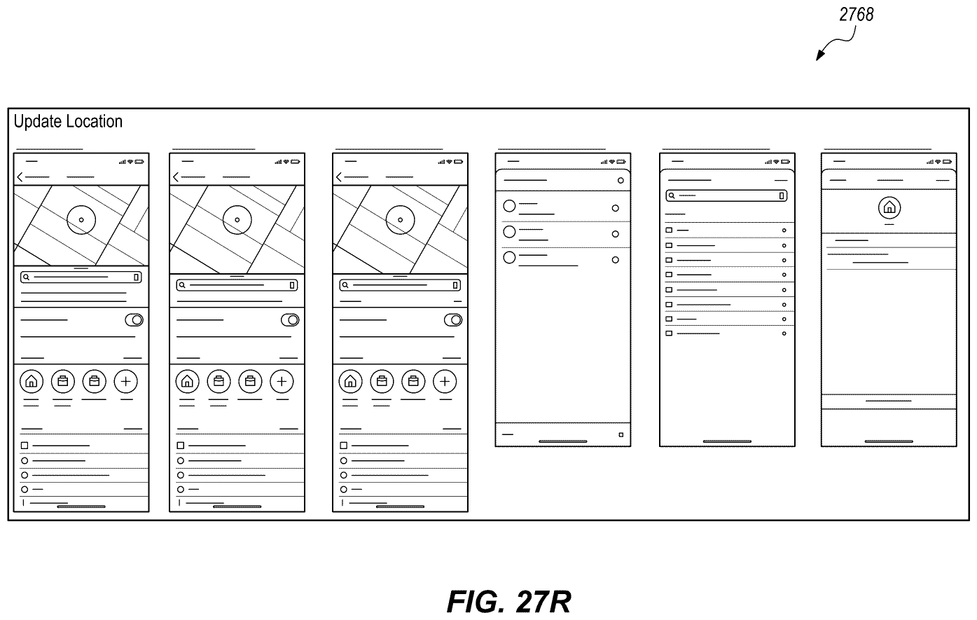

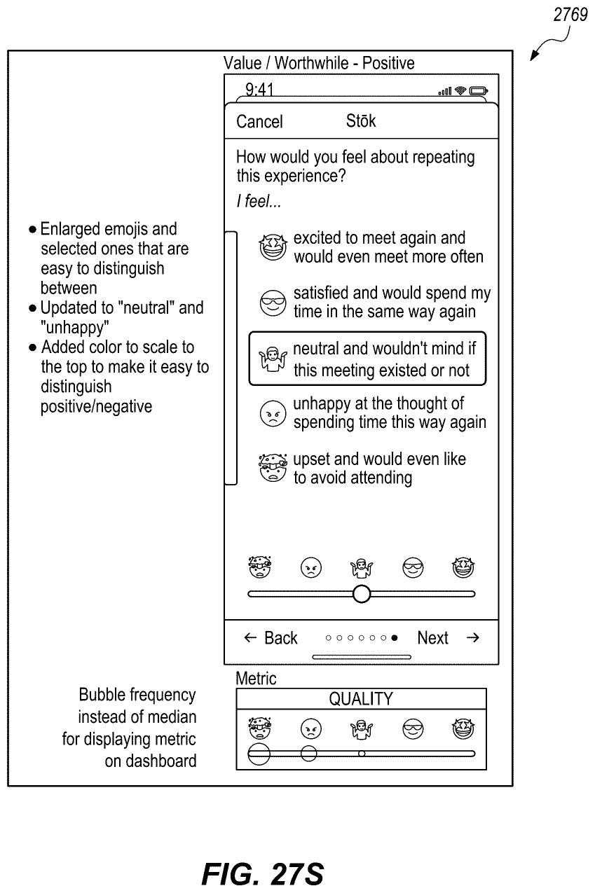

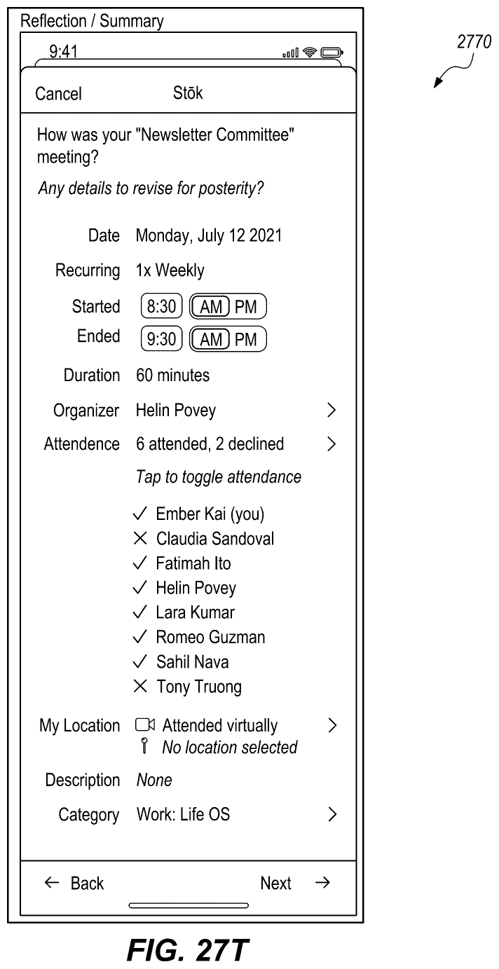

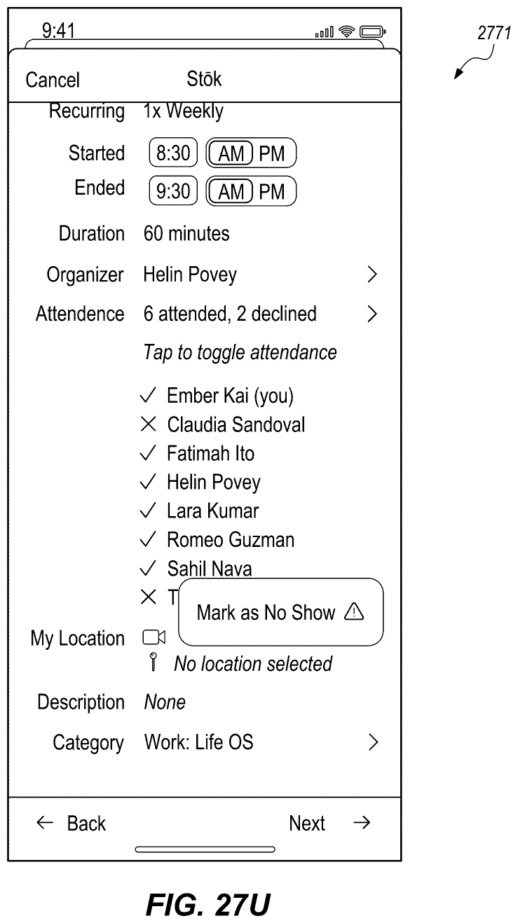

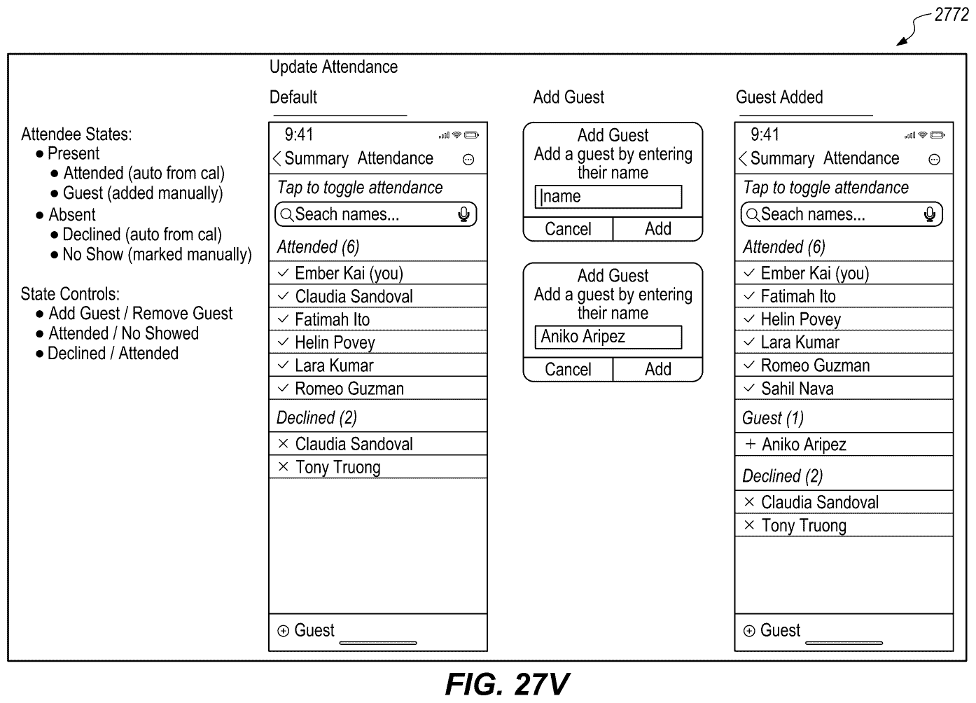

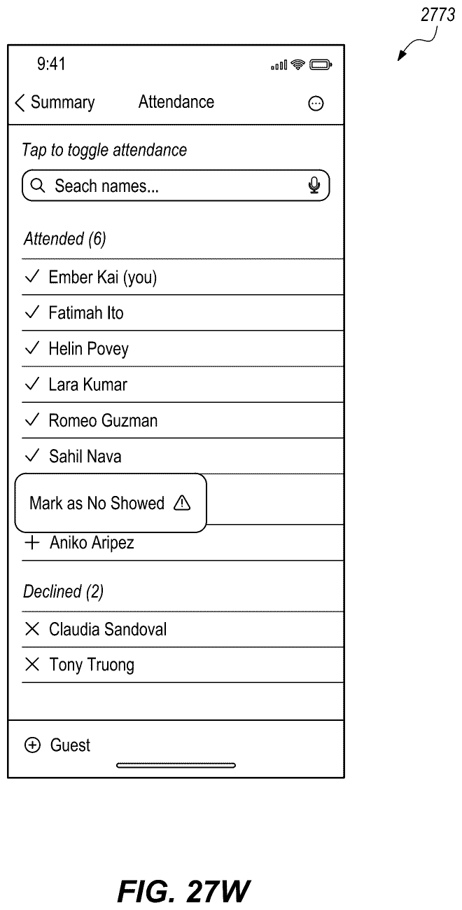

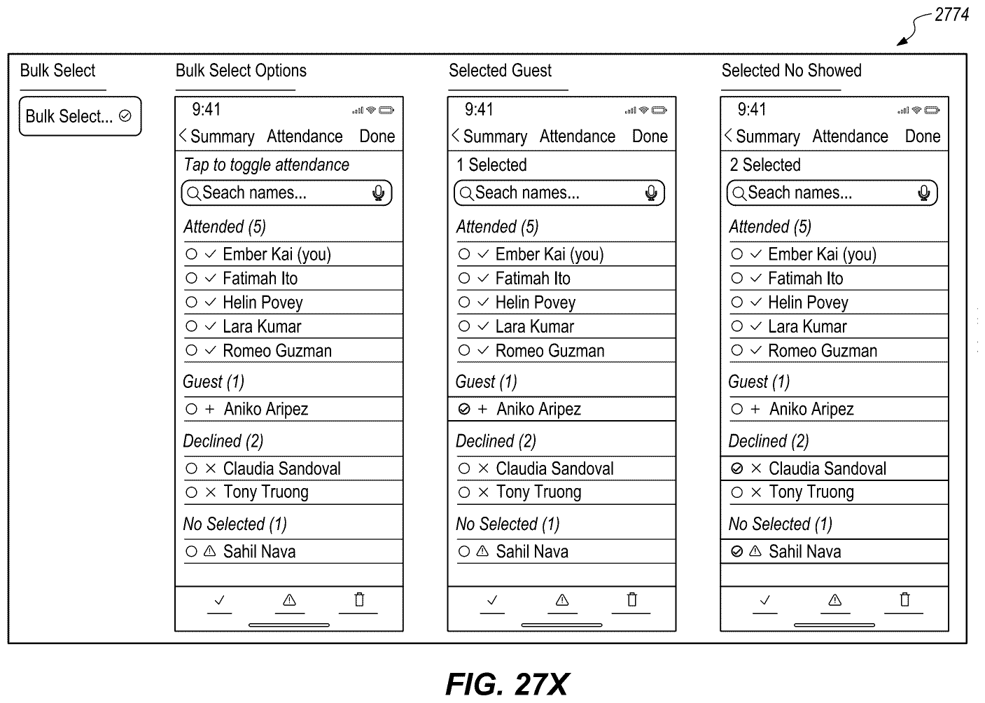

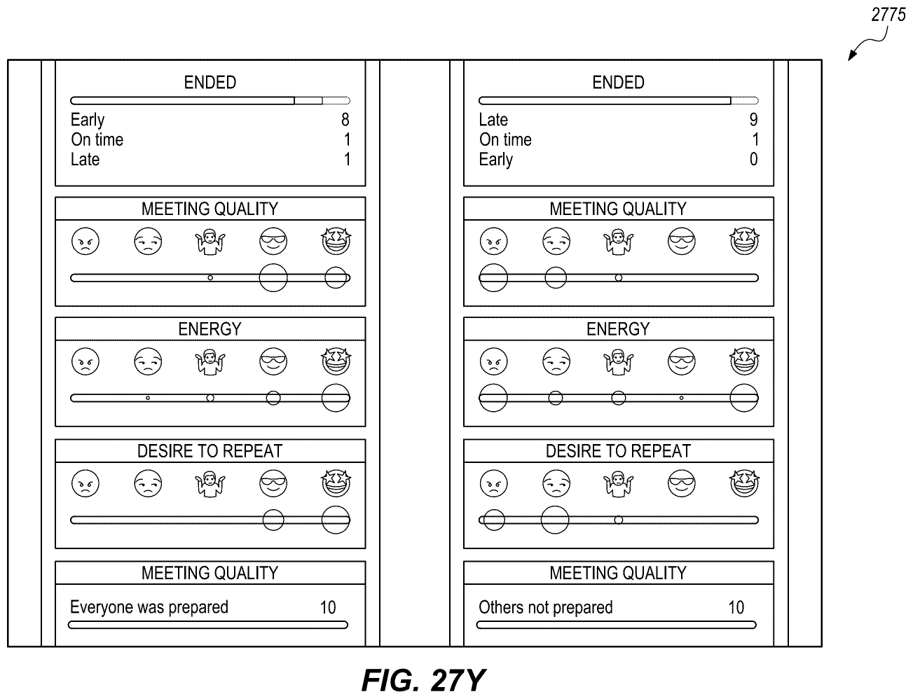

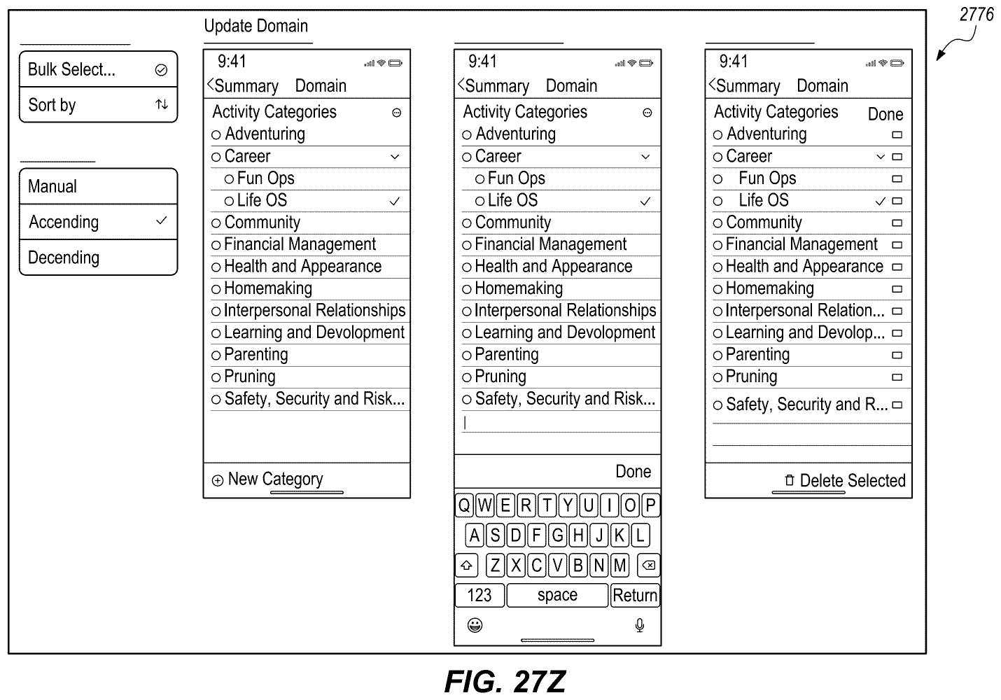

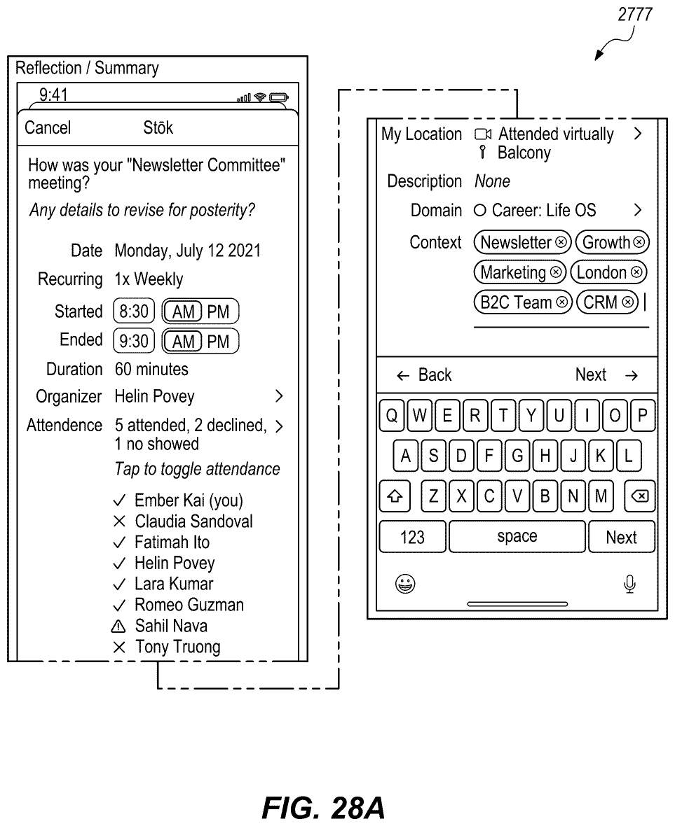

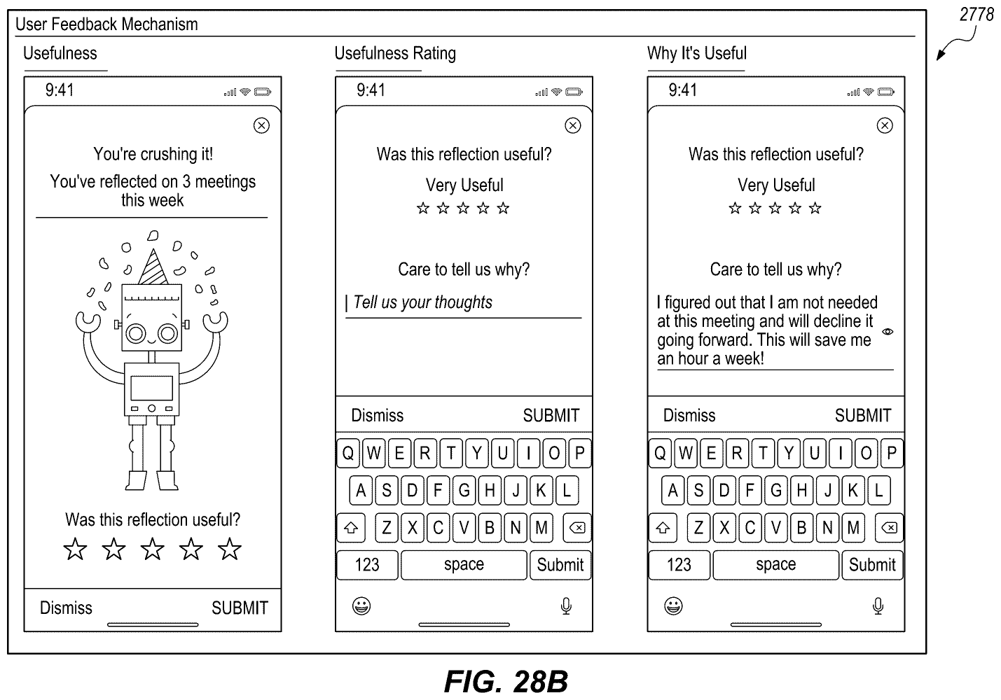

Referring now to FIGS. 27A through 27Z and FIGS. 28A through 28B, there are shown examples of various screens 2751-2778 that may be provided to enable functionality for meeting reflections according to one embodiment.

In at least one embodiment, any or all of the following elements may be included:
Notification screen 2751—Starting the flow by selecting a notification from the lock screen or Notifications tab.
Summary screen 2752—See a summary of the meeting pulled from the calendar invite. User 100 can also update the category of the event.
Quality screen 2753—Rate the overall quality of the meeting with a scale.
Intentions to Outcomes screens 2754, 2755—Y/N if intention/hopes match the outcomes. Tags describing outcome with some quick select suggestions.
Energy screen 2756—Allows user 100 to rate their energy during the meeting on a scale.
Reflection screen 2757—A mini-journal free text about what user 100 would do differently and how this time could have been better used.
Value screen 2758—A summary of the time calculations per day/week/months/year with Y/N if this meeting is worthwhile.
Additional feedback screens 2759-2765, 2767, 2769-2770, 2775, 2777.
Update Organizer screen 2766.
Update Location screen 2768.
Update Attendance screens 2771-2774.
Update Domain screen 2776.
User Feedback Mechanism screen 2778.
Several of the screens provide functionality to allow user 100 to indicate recommended actions to be taken, that may pop open within other apps, such as for example a calendar event showing in a calendar app to reduce recurrence.

### Practices

Referring now to FIGS. 26G, 26H, and 281, there are shown examples of various screens that may be associated with functionality for installing and configuring a practice according to one embodiment.

### Concepts

In at least one embodiment, the main configuration elements for practices may be supported by a wizard to help users 100 quickly install and configure the relevant functionality. Once the main configurations have been set, user 100 or FDE 251 has the option to adjust the practice to their needs.

For illustrative purposes, the examples depicted FIGS. 27, 28A, and 28B demonstrate screens to allow user 100 to install and experience a proven practice created by an expert. They can then incorporate this practice into their lives, experiment with different options, and track their progress over time.

Referring now to FIG. 26G there are shown examples of screens for installing a new practice.

### Install Practice Screen 2700

Screen 2700 allows user 100 to installing a new practice, so that they can to experiment with new avenues of achievement.

Summary section 2701 helps user 100 determine if this practice is interesting and relevant to their desires, and may include the following elements:
Image: cover of a book or other representation of the practice.
Resources: links to where the practice is derived from.
Info: blurb that advertises the benefits of the practice.
Duration: approximate amount of time the practice should take to complete (but does not include any supporting routines).
Recurring: recommended pattern for when the practice should be repeated.
Tools: recommended items necessary to complete the practice with best results.
Cadence and #of Reps: recommended manner in which this particular type of practice should be performed.

Configure section 2702 helps user 100 plan the steps necessary to make their practice a success, and may include the following elements:
Schedule: find and select the optimal day(s) and time(s) for them to perform their practice.
Supporting Routine: accurately plan what other actions they need to take in order to perform their practice well and with enough time.
Workout: recommended steps that are particular to the practice.
Next Practice: based on the schedule selected, this displays the next practice date and time.
Practice Duration: based on the supporting routine and workout durations, this displays the total estimated time.

Track Your Progress section 2703 allows user 100 to document what they want to achieve, how they want to measure their progress, track the context of the outcomes of their experiments, and what they want the system to push them for, and may include the following elements
Goals & Success Metrics
Reflections
Nudges

### Schedule Screen 2710

Screen 2710 helps user 100 find the optimal time in their schedule to perform their practice.

Configure section 2711 includes the following options:
Schedule: Toggle on for maintaining a schedule for this practice. User 100 may decide to turn it off to pause the recurring events in their schedule.
Add to weekly planning list: This reminds user 100 to factor this into their schedule when doing their weekly planning.
Location: User 100 can search for and select where they want to complete their practice.

Preferences section 2712 includes settings that allow user 100 to tell the system what they want the system to consider when finding options for scheduling their practice. This can function like filters for the schedule results below. Section 2712 may include controls for the following:
The Time of Day control provides access to screen 2730, which allows users 100 to configure practices during a specific time of day. For example, user 100 might know that they do better with exercising early in the morning before being bogged down by the rest of their day. This section allows user 100 to set custom hours or select the times of day they want to consider. If the custom hours toggle is on, user 100 is able to specify a time range, including for example Morning, Afternoon, and Evening. Time range options may be based on the start times set in App Settings. User 100 may have the option to edit those settings by following the link.
The Max Crowd Level control provides access to screen 2740, which allows users 100 to take into consideration how crowded an area might be before going there. User 100 can set the Max Crowd Level of the location they would like to take into consideration when suggesting times. The system may then only propose available times in user's 100 schedule where crowd levels are at or below the specified maximum. The maximum may be set by sliding across or touching the bars.

Schedule section 2713 helps user 100 determine the best day and time for them to perform their practice, as follows:
Day: Each row may be separated by day of the week and date. The first row may be toggled open by default. In response to user 100 tapping another day, that new row is toggled open and the previous one closed. The default selection is the first day and time proposed. The next 7 days are displayed.
Available: In at least one embodiment, any proposed time must be able to accommodate the estimated practice duration (i.e., supporting routine+workout). The earliest possible start time that matches all criteria may be displayed. Start time can be edited for that day manually. User 100 can see their agenda for the day and how close the practice comes to other events.
Crowd Levels: Estimated crowd levels may be pulled in from Google Maps and/or other sources.
The purple bar indicates the levels for the time selected so user 100 can compare it with the crowd levels for the rest of the day.

### Select Location Screens 2720A, 2720B

User 100 can search for and select the location at which they want to perform their practice. The system can estimate travel times and crowd levels based on the location. If desired, user 100 can add the location to their favorites.

In at least one embodiment, once a location has been selected, it shows up in the recents list.

### Supporting Routine

Referring now to FIG. 26H, there are shown examples of screens for selecting and configuring a supporting routine. User 100 can choose to have a supporting routine for accomplishing their practice with better predictability and ease.

Routine Steps screen 2800 can be configured by FDE 251 for user 100 to match their particular context. A set of routine steps may be recommended based on the specific practice. User 100 can adjust the amount of time they think they will need for each routine step, as follows:
Get Ready is an example of a routine with notes in addition to the time duration.
Travel Time may be added to the routine by default when a location for the practice is set.
Workout takes user 100 to the practice settings for Workout. This may vary depending on the practice.
Shower helps user 100 factor in any grooming after physical activity.
Buffer helps user 100 give themselves some wiggle room for any unexpected items that they can later factor into their routine.
Workout+Routine Duration helps user 100 to improve their intuition about how long it might actually take for them to complete their practice so they properly allocate the time they need and have a good experience performing it.

Add New Step screen 2810 may be displayed when user 100 taps on “New Step.” User 100 may be presented with options to:
Select one of their saved steps
Edit the details of a saved step
Create a custom step

In response to user 100 tapping on the “i” icon next to a saved step, Step Detail screen 2820 may be displayed. User 100 may be presented with options to update:
Icon (pick from emoji)
Label (default “Title”)
Duration (default 5 minutes)
Notes (default empty)

In response to user 100 tapping on “Remove Saved Step,” the step is removed from the list of Saved Steps, but is not removed from any existing Practices.

Travel Time To & From screens 2830, 2840 allow user 100 to select a Starting Location, which updates Travel Time estimates below. For Travel Time From Location, user 100 has the option to select a Destination that is different from their original starting location. Destination may be pulled from the Practice Location. User 100 may be presented with options to select travel time based on estimates for different modes of travel:
Car
Train
Walking

User 100 may also be presented with options to select from pre-set times or to add a custom time.

### Workout

Referring now to FIG. 26I, there are shown examples of screens for selecting and configuring a workout. This workout is an example of a specific type of practice. Similar to the Supporting Routine, user 100 is able to manage all of the steps.

In at least one embodiment, Workout Steps screen 2850 may include the following:
Configured section 2851 including:
   Tools specific to this practice, including an option to select between Machines and Free-Weights. This alters the exercise steps recommended (e.g., Seated Row vs. Bent-Over Barbell Row). Screen 2860 depicts an example of controls for configuring the Tools.
   Playlist, which may be a practice-dependent option allowing user 100 to select the music they want to listen to when they are performing the practice.
Workout Steps section 2852 including various exercises and warm up and cool down periods. Users 100 are able to adjust the time they want to allocate to each. Workout Duration estimates how long the workout will take. User 100 can rearrange and nest steps by dragging the row.
Add New Step button 2853, which provides access to screen 2870 for adding a step from Saved Steps 2871 by tapping on a row 2872. User 100 can see the details of a saved step by tapping on “i” icon 2873, or can create a custom step by tapping on “Custom Step” 2874.

Create Custom Step & Edit Details screen 2880 allows user 100 to edit the details of a new custom step or an existing step. User 100 can select among the following:
Icon (pick from emoji)
Label (default “Title”)
Duration (default 5 minutes)
Notes (default empty)

In response to user 100 tapping on “Remove Saved Step,” the step is removed from the list of Saved Steps, but is not removed from any existing Practices.

### Data Sharing

In at least one embodiment, the described system may provide mechanisms to allow users 100 to share data with one another. The purpose of sharing is to enable user 100 to give other people continuous, interactive access to a portion of their life's data while still being able to control the parameters of the data being shared, eliminating the need to create and send recurring reports.

In at least one embodiment, user 100 may send data describing their life to different audiences at varying levels of access. For example,
User 100 may wish to share important, contextualizing data with the experts so that they may be better able to assist user 100.
User 100 may wish to make knowledge available to important people in their life so as to provide better shared awareness, coordination, and continuity.
User 100 may wish share context and experiences with people they care about so that they can feel seen/understood and create better rapport.

The system may provide static datashares to enable user 100 to allow other people to access a summarized snapshot of a portion of their data from a specific date range. A static datashare is similar to a report and is easier to define the limits of the data being shared.

The system may provide dynamic datashares to enable user 100 to allow other people interactive access on various apps to a portion of their data from the date range they set. With dynamic shares, user 100 can share their data continuously, eliminating the need to send recurring reports.

In at least one embodiment, data shares can be shared for a limited period of time, and can selectively contain data from a limited period of time. In at least one embodiment, if user 100 shares their data with another user, the receiving user can access the sharer's data from their own timeline 300, for example by quickly switching between data sources.

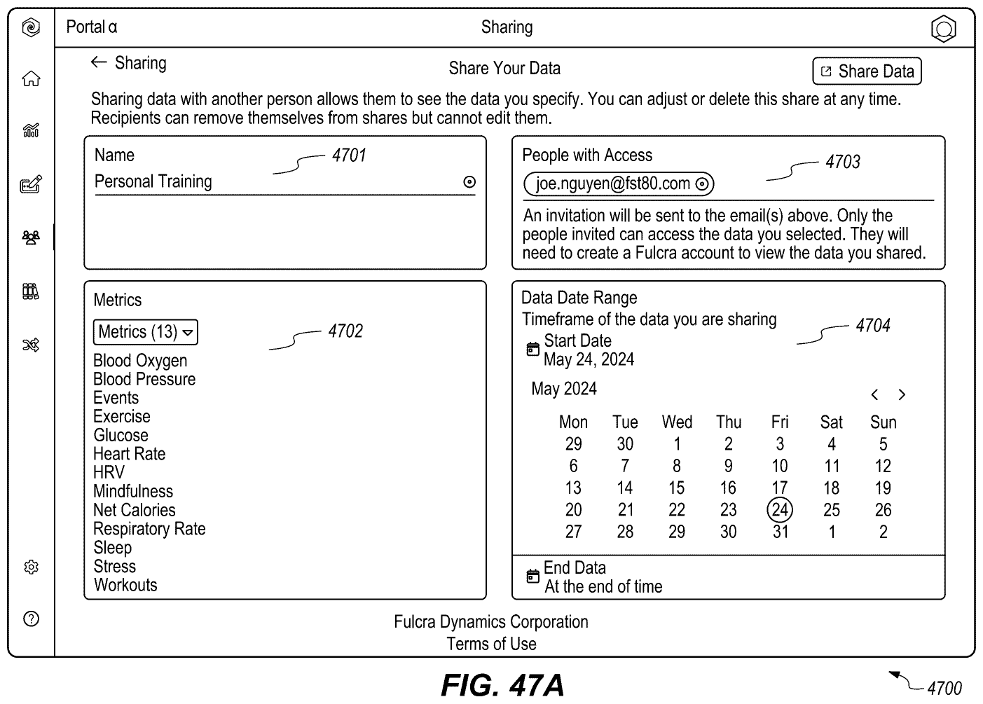

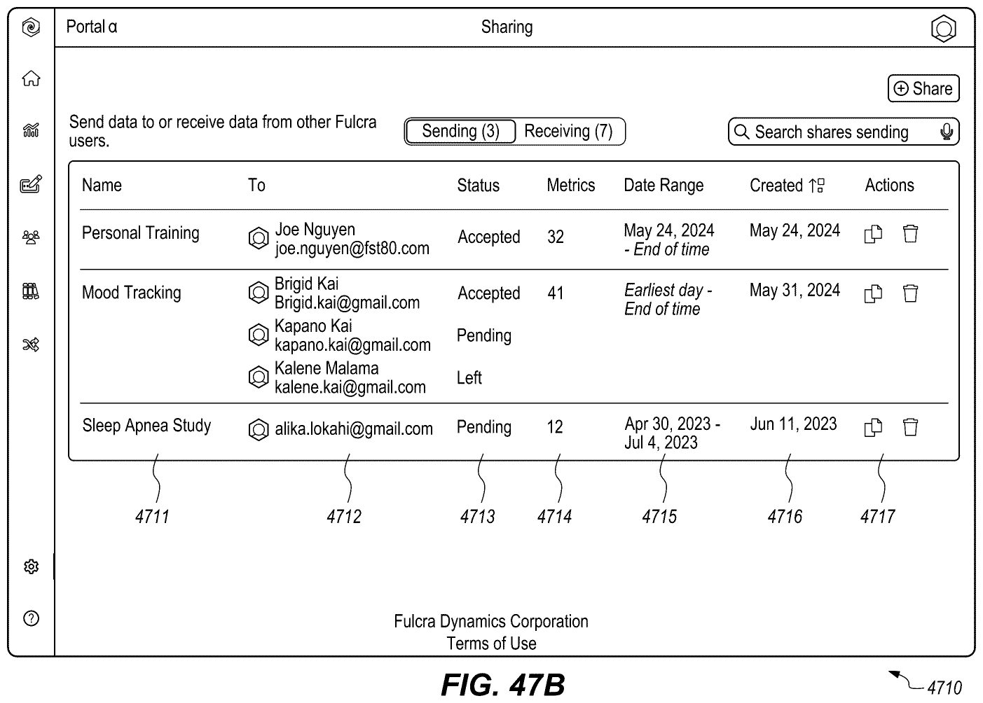

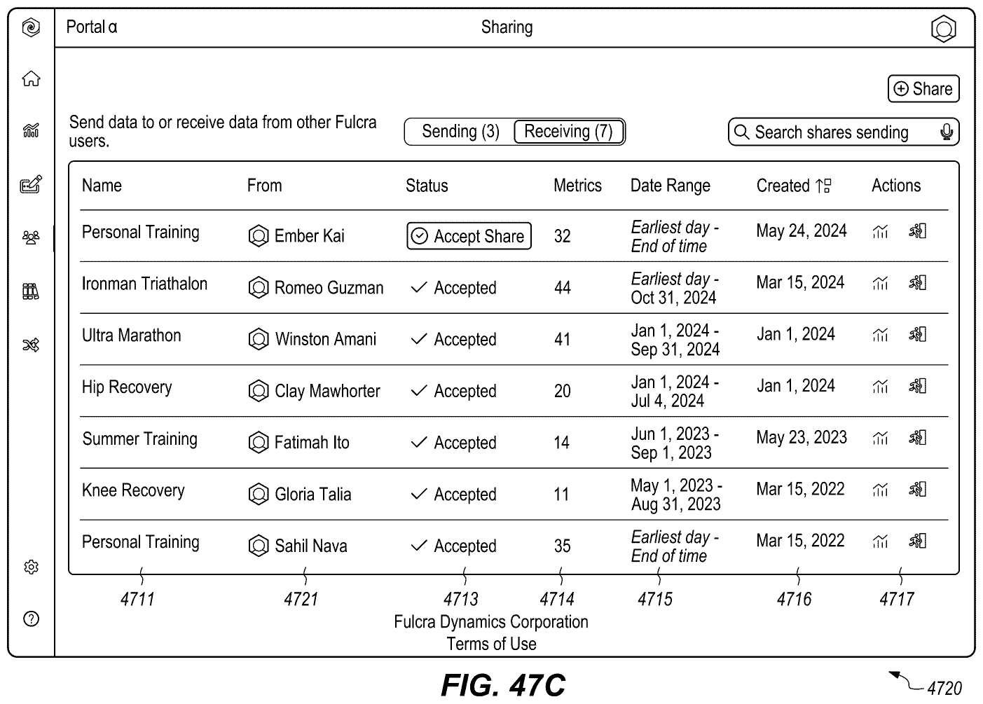

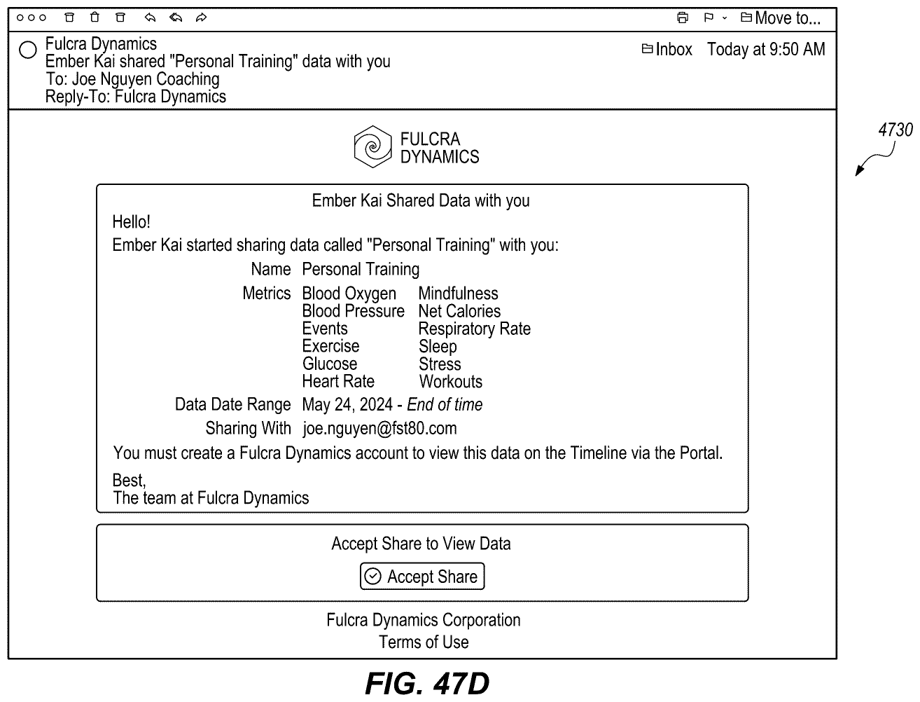

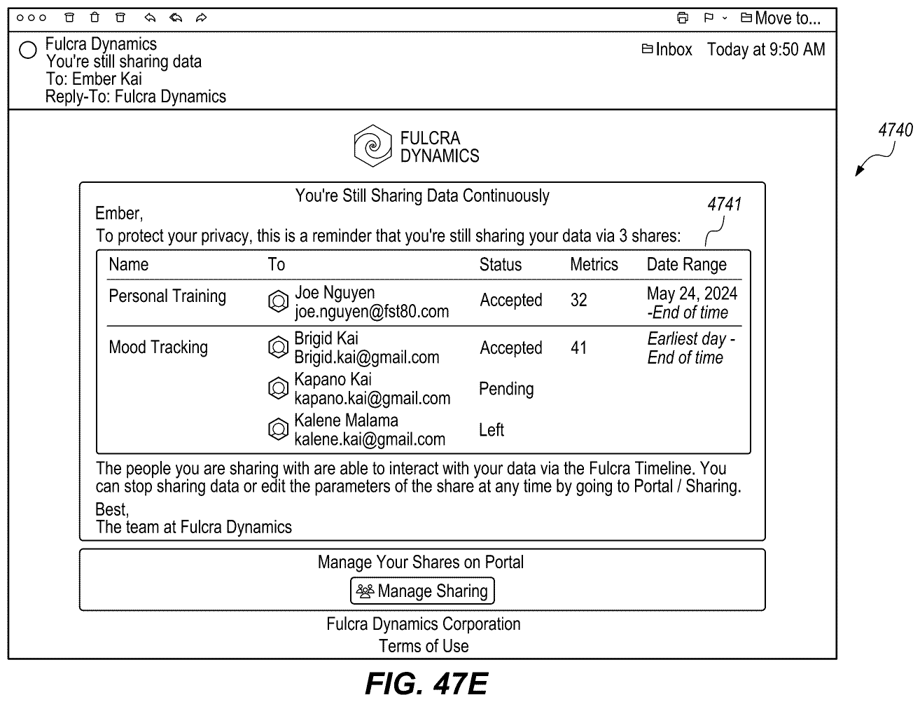

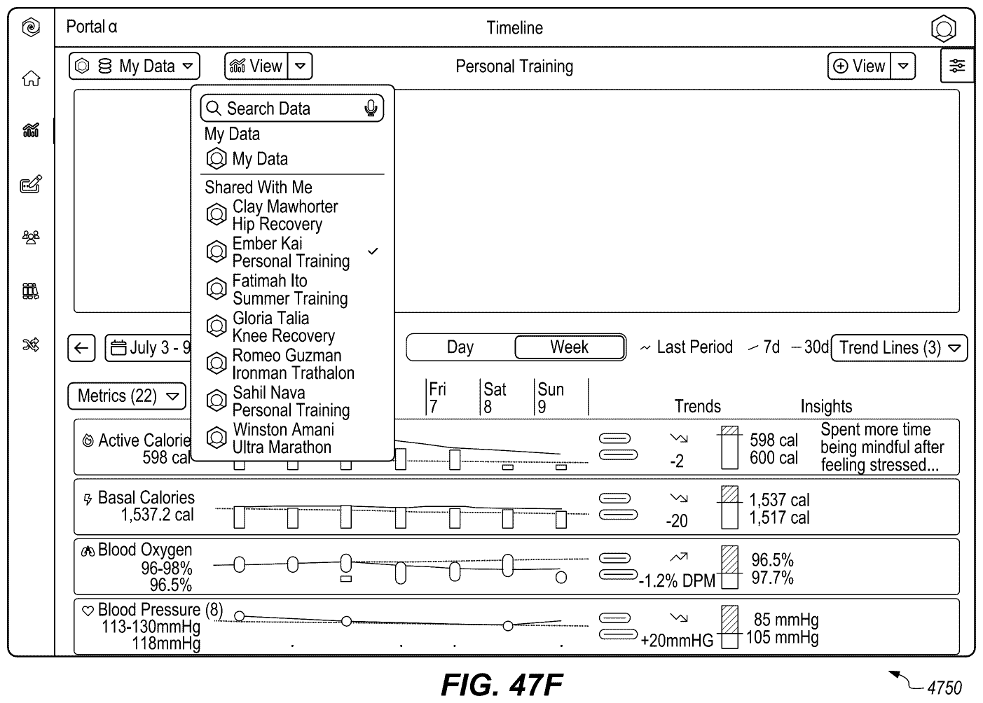

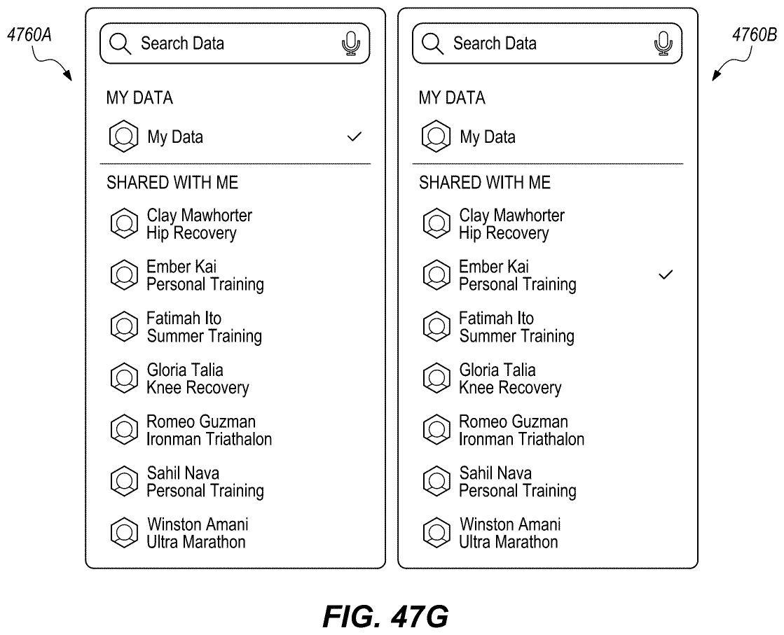

Referring now to FIGS. 47A through 47G, there are shown examples of screens for creating and managing shares according to one embodiment.

FIG. 47A depicts an example of a Sharing screen 4700 that may be displayed when user 100 clicks on a new share button on a Sharing page. Screen 4700 allows user 100 to configure the share.

Name field 4701 allows user 100 to name the share.

Metrics pane 4702 allows user 100 to select metrics they would like to share.

People with Access pane 4703 allows user 100 to specify who should have access to the share. These may be provided, for example, via email addresses. Addressees may receive email invites when they have been included in a share.

Date Range pane 4704 allows user 100 to select a data range fort he shared data and/or expiration date for the share.

FIG. 47B depicts an example of a Manage Share screen 4710 for managing existing shares that user 100 is sending. For each share being sent, the following information may be presented:
name 4711;
recipient 4712;
status 4713 such as, for example, pending, accepted, or left (indicating that the receiver has left the share);
number of metrics 4714 (in at least one embodiment, hovering over this value shows a list of the metrics being shared);
date range 4715;
date created 4716.

Actions button 4717 provides access to actions such as copying the share, modifying it, and/or deleting it.

FIG. 47C depicts an example of a Manage Share screen 4720 for managing existing shares that user 100 is receiving. For each share being received or pending, the following information may be presented:
name 4711;
sender 4721;
status 4713 (if pending, user 100 can click here to accept or decline the share);
number of metrics 4714 (in at least one embodiment, hovering over this value shows a list of the metrics being shared);
date range 4715;
date created 4716.

Actions button 4717 provides access to actions such as copying the share, leaving it, and/or the like.

In at least one embodiment, when a share is accepted, the recipient may receive a confirmation email. FIG. 47D depicts an example of confirmation email 4730.

In at least one embodiment, the person sharing data may receive periodic reminders that they are still sharing data. FIG. 47E depicts an example of reminder email 4740. In at least one embodiment, table 4741 showing a summary of the shares may be included. A link to a manage share screen such as screen 4710 may be provided, allowing user 100 to modify or delete the share.

In at least one embodiment, shared data may be presented on timeline 300. FIG. 47F depicts an example of screen 4750 wherein shared data is shown on timeline 300. Various settings may be provided, including date range, view level, selected metrics, metric order, and/or trend lines.

In at least one embodiment, user 100 can select among various sources of shared data. FIG. 47G depicts examples of screens 4760A, 4760B for selecting among shared sources. In screen 4760A, user 100 has selected their own data. In screen 4760B, user 100 has selected another person's data. In at least one embodiment, user 100 can select multiple sources to be viewed simultaneously on a unified display.

### Connections

In at least one embodiment, a Custom Input Endpoint connection may be provided, to allow users 100 to create an endpoint for setups like physical buttons or iOS shortcuts that records custom inputs into their data store 106. User 100 may configure the connection and save it, thus allowing data collected via the connection to be shown within timeline 300.

In at least one embodiment, a Manage Connections page may be provided, to allow user 100 to make new connections and manage existing connections place. Data endpoints may be distinguished from data sources as connections, separate from data sharing, which allows user 100 to send data externally data store 106.

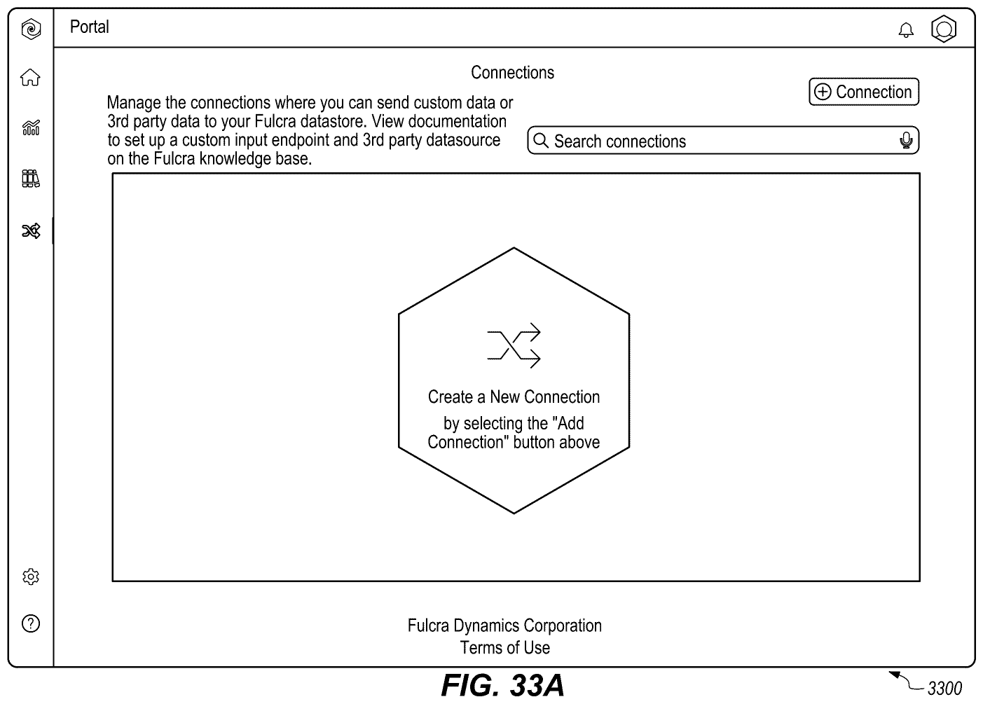

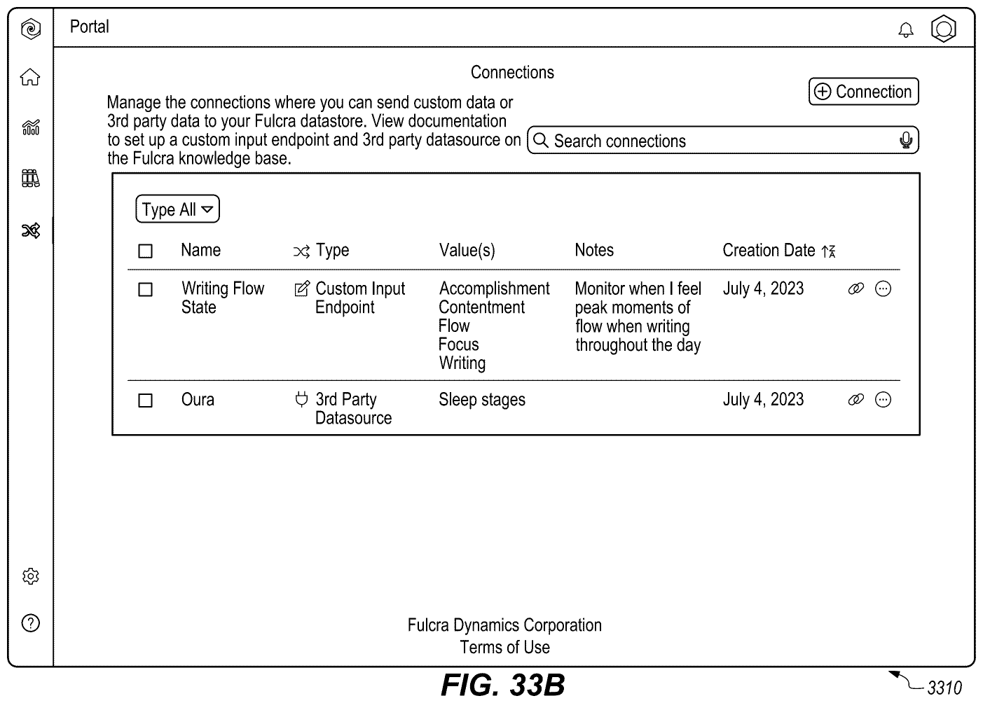

Referring now to FIG. 33A, there is shown an example of a Manage Connections screen 3300.

Referring now to FIG. 33B, there is shown an example of a Connections Details screen 3310.

### Library

### Data Schemas

In at least one embodiment, the system allows for creating and applying data schemas. The purpose of a data schema is to allow users 100 to assign custom header names to the columns displayed in their dataset so that it is easy to understand/reference data on their Timeline 300, analyze their data, and set up visualizations.

Once a schema is created, it is easier to apply the same schema consistently across multiple datasets without having to make local adjustments in each file. Updating a schema updates the display of the data columns for all the files to which that schema is applied, making it easier to update multiple datasets at once.

In at least one embodiment, a user 100 may create a schema by using a starter schema that they can adjust to their liking.

User 100 may select a base schema, and may include a number of columns in the schema. User 100 may then define the type of data that the column contains to ensure that it is properly processed by the data service. A predicted data type is selected based on the data that was parsed from the base file. User 100 may adjust the data type as necessary by making a new selection from a data type dropdown.

In at least one embodiment, once one or more schemas have been created, user 100 may apply a data schema to multiple files or a folder of files, so as to allow user 100 to apply the same schema consistently across multiple datasets without having to make local adjustments in each file.

In at least one embodiment, user can apply schemas to files and folders via two methods:
Bulk Selection
Single Selection

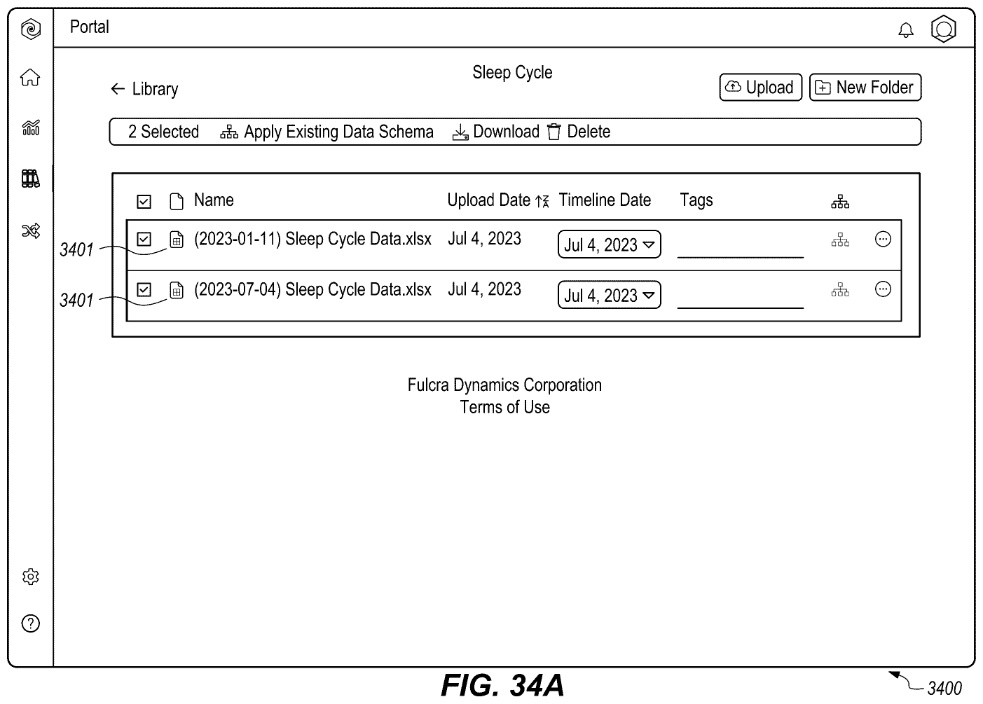

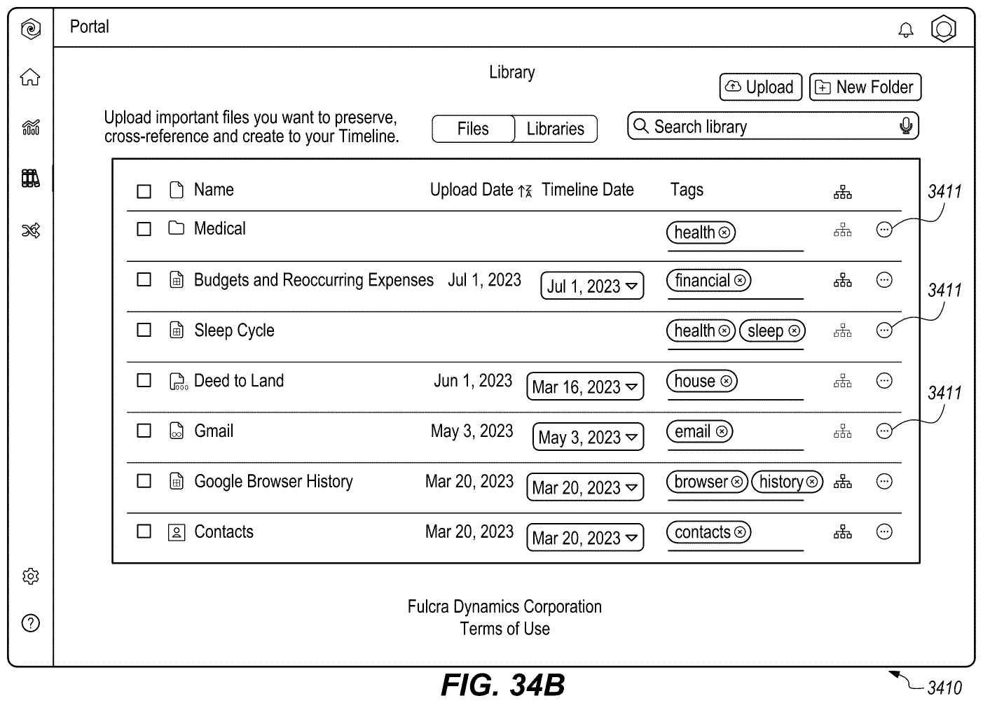

Referring now to FIG. 34A, there is shown an example of a Bulk Selection screen 3400. Here, user 100 has selected multiple files 3401 to which they can perform the actions of applying an existing data schema, download, or delete

Referring now to FIG. 34B, there is shown an example of a Single Selection screen 3410, allowing user 100 to view and manage the files in their library. To perform actions to individual items, they can click on “more” icon 3411 to display the menu of actions listed above

User 100 may also manage existing schemas, including editing and/or deleting them.

### Upload and Manage Files

In at least one embodiment, a Files tab may be provided to allow user 100 to preserve their important data and documents; have their data normalized and made accessible in a machine-readable format for analysis; and cross-reference/relate their data and documents on their Timeline 300.

In at least one embodiment, all files uploaded are preserved in user's 100 data store 106 within the system.

### Edit Profile

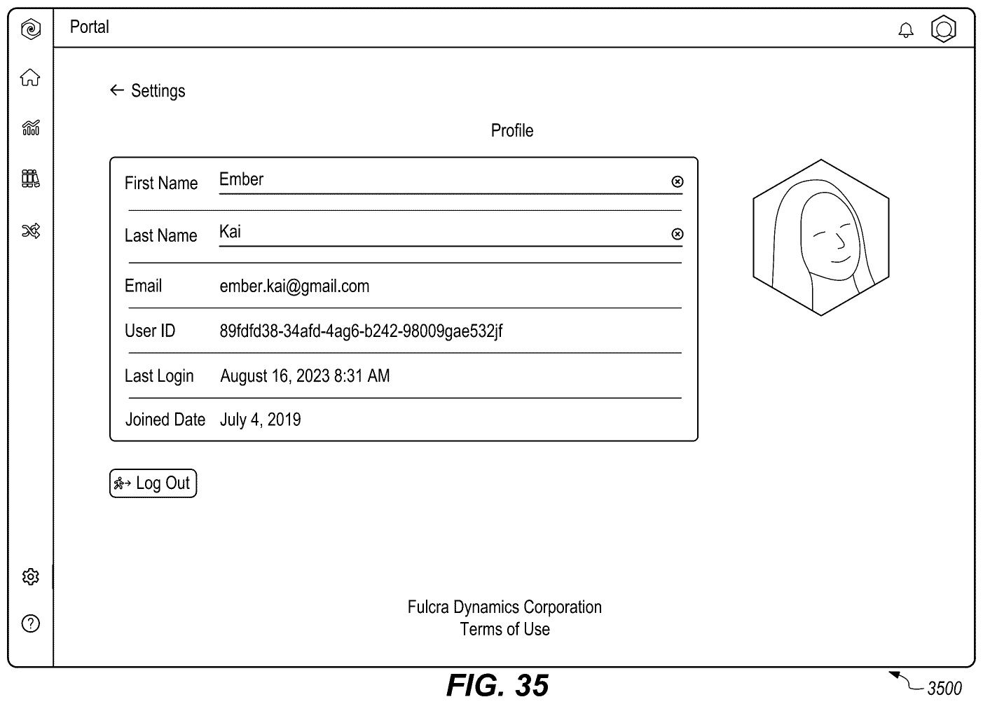

Referring now to FIG. 35, there is shown an example of a profile page 3500 according to one embodiment. Profile page 3500 may provide a way for user 100 to view and edit personal details related to their account. In at least one embodiment, user's 100 profile information may be generated based on an existing online account, such as a Google account. User 100 may use profile page 3500 to edit various details about their profile.

### Select Calendars

In at least one embodiment, the system may allow user 100 to select among different available calendars, so as to filter out unwanted calendar information from their results and/or data store 106.

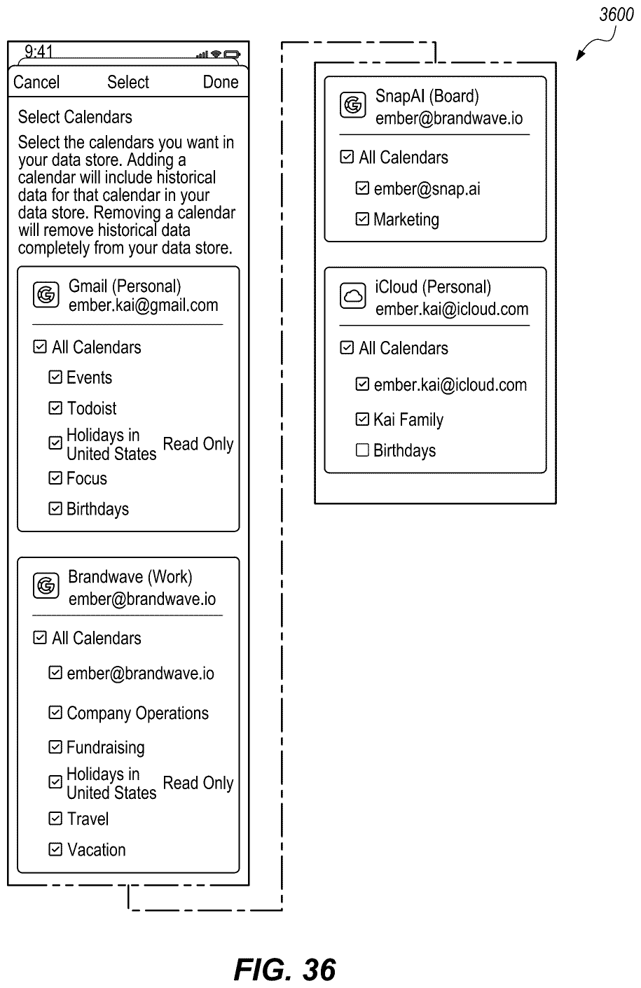

Referring now to FIG. 36, there is shown an example of a Select Calendars screen 3600.

### Time Zone

In at least one embodiment, the system may allow user 100 to select a default time zone in which they wish to view their data. Automatic time zone adjustment may be supported, which allows user 100 to view their activities as they happen while the user may be traveling to different time zones.

### Bulk Actions

In at least one embodiment, the system allows user 100 to select multiple metric rows on which to perform an action, and/or to rearrange the order of metric rows.

The user can select rows on which actions are to be performed by clicking on a button to the left of each row. Selected rows may then be hidden, rearranged, reordered, and/or otherwise edited or manipulated.

The present system and method have been described in particular detail with respect to possible embodiments. Those of skill in the art will appreciate that the system and method may be practiced in other embodiments. First, the particular naming of the components, capitalization of terms, the attributes, data structures, or any other programming or structural aspect is not mandatory or significant, and the mechanisms and/or features may have different names, formats, or protocols. Further, the system may be implemented via a combination of hardware and software, or entirely in hardware elements, or entirely in software elements. Also, the particular division of functionality between the various system components described herein is merely exemplary, and not mandatory; functions performed by a single system component may instead be performed by multiple components, and functions performed by multiple components may instead be performed by a single component.

Reference in the specification to “one embodiment” or to “an embodiment” means that a particular feature, structure, or characteristic described in connection with the embodiments is included in at least one embodiment. The appearances of the phrases “in one embodiment” or “in at least one embodiment” in various places in the specification are not necessarily all referring to the same embodiment.

Various embodiments may include any number of systems and/or methods for performing the above-described techniques, either singly or in any combination. Another embodiment includes a computer program product comprising a non-transitory computer-readable storage medium and computer program code, encoded on the medium, for causing a processor in a computing device or other electronic device to perform the above-described techniques.

Some portions of the above may be presented in terms of algorithms and symbolic representations of operations on data bits within a memory of a computing device. These algorithmic descriptions and representations may be the means used by those skilled in the data processing arts to convey the substance of their work most effectively to others skilled in the art. An algorithm is here, and generally, conceived to be a self-consistent sequence of steps (instructions) leading to a desired result. The steps may be those requiring physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “displaying” or “determining” or the like, refer to the action and processes of a computer system, or similar electronic computing module and/or device, that manipulates and transforms data represented as physical (electronic) quantities within the computer system memories or registers or other such information storage, transmission or display devices.

Certain aspects include process steps and instructions described herein in the form of an algorithm. It should be noted that the process steps and instructions may be embodied in software, firmware and/or hardware, and when embodied in software, may be downloaded to reside on and be operated from different platforms used by a variety of operating systems.

The present document also relates to an apparatus for performing the operations herein. This apparatus may be specially constructed for the required purposes, or it may comprise a general-purpose computing device selectively activated or reconfigured by a computer program stored in the computing device. Such a computer program may be stored in a computer readable storage medium, such as, but is not limited to, any type of disk including floppy disks, optical disks, CD-ROMs, DVD-ROMs, magnetic-optical disks, read-only memories (ROMs), random access memories (RAMs), EPROMs, EEPROMs, flash memory, solid state drives, magnetic or optical cards, application specific integrated circuits (ASICs), or any type of media suitable for storing electronic instructions, and each coupled to a computer system bus. Further, the computing devices referred to herein may include a single processor or may be architectures employing multiple processor designs for increased computing capability.

The algorithms and displays presented herein are not inherently related to any particular computing device, virtualized system, or other apparatus. Various general-purpose systems may also be used with programs in accordance with the teachings herein, or it may prove convenient to construct more specialized apparatus to perform the required method steps. The required structure for a variety of these systems will be apparent from the description provided herein. In addition, the system and method are not described with reference to any particular programming language. It will be appreciated that a variety of programming languages may be used to implement the teachings described herein, and any references above to specific languages are provided for disclosure of enablement and best mode.

Accordingly, various embodiments include software, hardware, and/or other elements for controlling a computer system, computing device, or other electronic device, or any combination or plurality thereof. Such an electronic device may include, for example, a processor, an input device (such as a keyboard, mouse, touchpad, microphone, and/or any combination thereof), an output device (such as a screen, speaker, and/or the like), memory, long-term storage (such as magnetic storage, solid state storage, and/or the like), and/or network connectivity, according to techniques that may be well known in the art. Such an electronic device may be portable or non-portable. Examples of electronic devices that may be used for implementing the described system and method include: a smartphone, kiosk, server computer, enterprise computing device, desktop computer, laptop computer, tablet computer, consumer electronic device, or the like. An electronic device may use any operating system such as, for example and without limitation: Linux; Microsoft Windows, available from Microsoft Corporation of Redmond, Washington; MacOS, available from Apple Inc. of Cupertino, California; iOS, available from Apple Inc. of Cupertino, California; Android, available from Google, Inc. of Mountain View, California; and/or any other operating system that is adapted for use on the device.

While a limited number of embodiments have been described herein, those skilled in the art, having benefit of the above description, will appreciate that other embodiments may be devised. In addition, it should be noted that the language used in the specification has been principally selected for readability and instructional purposes, and may not have been selected to delineate or circumscribe the subject matter. Accordingly, the disclosure is intended to be illustrative, but not limiting, of scope.

What is claimed is:

1. A computer-implemented method for displaying a unified timeline of user data, comprising:
at a hardware processor, establishing connections with a plurality of data sources;
at the hardware processor, via the established connections, receiving time-varying data associated with a user; and
on a display device, displaying a unified timeline along an axis, wherein the unified timeline depicts at least a portion of the time-varying data.

2. The method of claim 1, wherein the plurality of data sources comprises at least one data source that is remotely located with respect to the hardware processor.

3. The method of claim 1, wherein the plurality of data sources comprises at least one wearable device for monitoring at least one metric associated with the user.

4. The method of claim 1, wherein the unified timeline is interactive.

5. The method of claim 1, further comprising:
at a user input device, receiving user input to manipulate the displayed timeline; and
on the display device, modifying the displayed timeline according to the received user input.

6. The method of claim 1, further comprising:
at a user input device, receiving user input to specify a date range;
and wherein displaying the unified timeline comprises displaying data corresponding to the specified date range.

7. The method of claim 1, wherein the time-varying data comprises data describing at least one selected from the group consisting of:
activity data for the user;
sleep data for the user;
net calorie intake and consumption for the user; and
user behavior.

8. The method of claim 1, wherein the time-varying data comprises data describing at least one selected from the group consisting of:
activity data for the user;
sleep data for the user;
screen time for the user;
purchasing behavior of the user;
net calorie intake and consumption for the user; and
user behavior.

9. The method of claim 1, wherein establishing connections with a plurality of data sources comprises providing application programming interfaces (APIs) for receiving data from the plurality of data sources.

10. The method of claim 1, further comprising securely storing the received data.

11. The method of claim 1, wherein displaying the unified timeline along an axis comprises:
displaying a playhead at a location along the axis, the location associated with a point in time;
displaying data associated with the point in time;
automatically moving the playhead along the axis to successive points in time; and
concurrently with automatically moving the playhead along the axis, updating the displayed data to reflect the current position of the playhead.

12. The method of claim 11, wherein updating the displayed data to reflect the current position of the playhead comprises continuously updating the displayed data to reflect the current position of the playhead.

13. The method of claim 11, further comprising:
receiving user input to move the playhead to a new point in time; and
updating the displayed data to reflect the current position of the playhead.

14. The method of claim 1, wherein the unified timeline depicts time-varying data associated with at least two different sources.

15. The method of claim 1, wherein the unified timeline depicts time-varying data associated with at least two different sources.

16. The method of claim 1, wherein displaying the unified timeline comprises displaying at least one event associated with the user.

17. A non-transitory computer-readable medium for displaying a unified timeline of user data, comprising instructions stored thereon, that when performed by a hardware processor, perform the steps of:
establishing connections with a plurality of data sources;
via the established connections, receiving time-varying data associated with a user; and
causing a display device to display a unified timeline along an axis, wherein the unified timeline depicts at least a portion of the time-varying data.

18. The non-transitory computer-readable medium of claim 17, wherein the plurality of data sources comprises at least one data source that is remotely located with respect to the hardware processor.

19. The non-transitory computer-readable medium of claim 17, wherein the plurality of data sources comprises at least one wearable device for monitoring at least one metric associated with the user.

20. The non-transitory computer-readable medium of claim 17, wherein the unified timeline is interactive.

21. The non-transitory computer-readable medium of claim 17, further comprising instructions stored thereon, that when performed by the hardware processor, perform the steps of:
causing a user input device to receive user input to manipulate the displayed timeline; and
causing the display device to modify the displayed timeline according to the received user input.

22. The non-transitory computer-readable medium of claim 17, further comprising instructions stored thereon, that when preformed by the hardware processor, perform the step of:
causing a user input device to receive user input to specify a date range;
and wherein causing the display device to display the unified timeline comprises causing the display device to display data corresponding to the specified date range.

23. The non-transitory computer-readable medium of claim 17, wherein the time-varying data comprises data describing at least one selected from the group consisting of:
activity data for the user;
sleep data for the user;
net calorie intake and consumption for the user; and
user behavior.

24. The non-transitory computer-readable medium of claim 17, wherein the time-varying data comprises data describing at least one selected from the group consisting of:
activity data for the user;
sleep data for the user;
screen time for the user;
purchasing behavior of the user;
net calorie intake and consumption for the user; and
user behavior.

25. The non-transitory computer-readable medium of claim 17, wherein establishing connections with a plurality of data sources comprises providing application programming interfaces (APIs) for receiving data from the plurality of data sources.

26. The non-transitory computer-readable medium of claim 17, further comprising instructions stored thereon, that when performed by the hardware processor, perform the step of causing a data storage device to securely store the received data.

27. The non-transitory computer-readable medium of claim 17, wherein causing the display device to display the unified timeline along an axis comprises:
causing the display device to display a playhead at a location along the axis, the location associated with a point in time;
causing the display device to display data associated with the point in time;
causing the display device to automatically move the playhead along the axis to successive points in time; and
concurrently with automatically moving the playhead along the axis, causing the display device to update the displayed data to reflect the current position of the playhead.

28. The non-transitory computer-readable medium of claim 27, wherein updating the displayed data to reflect the current position of the playhead comprises continuously updating the displayed data to reflect the current position of the playhead.

29. The non-transitory computer-readable medium of claim 27, further comprising instructions stored thereon, that when performed by the hardware processor, perform the steps of:
causing a user input device to receive user input to move the playhead to a new point in time; and
causing the display device to update the displayed data to reflect the current position of the playhead.

30. The non-transitory computer-readable medium of claim 17, wherein the unified timeline depicts time-varying data associated with at least two different data sources.

31. The non-transitory computer-readable medium of claim 17, wherein the unified timeline depicts time-varying data associated with at least two different data sources.

32. The non-transitory computer-readable medium of claim 17, wherein displaying the unified timeline comprises displaying at least one event associated with the user.

33. A system for displaying a unified timeline of user data, comprising:
a hardware processor, configured to establishing connections with a plurality of data sources and to receive, via the established connections, time-varying data associated with a user; and
a display device, communicatively coupled to the hardware processor, configured to display a unified timeline along an axis, wherein the unified timeline depicts at least a portion of the time-varying data.

34. The system of claim 33, wherein the plurality of data sources comprises at least one data source that is remotely located with respect to the hardware processor.

35. The system of claim 33, wherein the plurality of data sources comprises at least one wearable device for monitoring at least one metric associated with the user.

36. The system of claim 33, wherein the unified timeline is interactive.

37. The system of claim 33, further comprising:
a user input device, communicatively coupled to the hardware processor, configured to receive user input to manipulate the displayed timeline;
wherein the display device is further configured to modify the displayed timeline according to the received user input.

38. The system of claim 33, further comprising:
a user input device, communicatively coupled to the hardware processor, configured to receive user input to specify a date range;
and wherein displaying the unified timeline comprises displaying data corresponding to the specified date range.

39. The system of claim 33, wherein the time-varying data comprises data describing at least one selected from the group consisting of:
activity data for the user;
sleep data for the user;
net calorie intake and consumption for the user; and
user behavior.

40. The system of claim 33, wherein the time-varying data comprises data describing at least one selected from the group consisting of:
activity data for the user;
sleep data for the user;
screen time for the user;
purchasing behavior of the user;
net calorie intake and consumption for the user; and
user behavior.

41. The system of claim 33, wherein establishing connections with a plurality of data sources comprises providing application programming interfaces (APIs) for receiving data from the plurality of data sources.

42. The system of claim 33, further comprising a data storage device, communicatively coupled to the hardware processor, configured to securely store the received data.

43. The system of claim 33, wherein displaying the unified timeline along an axis comprises:
displaying a playhead at a location along the axis, the location associated with a point in time;
displaying data associated with the point in time;
automatically moving the playhead along the axis to successive points in time; and
concurrently with automatically moving the playhead along the axis, updating the displayed data to reflect the current position of the playhead.

44. The system of claim 43, wherein updating the displayed data to reflect the current position of the playhead comprises continuously updating the displayed data to reflect the current position of the playhead.

45. The system of claim 43, further comprising:
a user input device, communicatively coupled to the hardware processor, configured to receive user input to move the playhead to a new point in time;
wherein the display device is further configured to update the displayed data to reflect the current position of the playhead.

46. The system of claim 1, wherein the unified timeline depicts time-varying data associated with at least two different data sources.

47. The system of claim 33, wherein the unified timeline depicts time-varying data associated with at least two different data sources.

48. The system of claim 33, wherein displaying the unified timeline comprises displaying at least one event associated with the user.
